(19) United States
(12) Patent Application Publication
(10) Pub. No.: US 2011/0076272 A1
(43) Pub. Date: Mar. 31, 2011

(54) THERAPEUTIC METHODS AND COMPOSITIONS

(76) Inventors: Victoria Smith, Burlingame, CA (US); Peter Van Vlasselaer, Portola Valley, CA (US)

(21) Appl. No.: 12/860,838

(22) Filed: Aug. 20, 2010

Related U.S. Application Data
(60) Provisional application No. 61/235,852, filed on Aug. 21, 2009.

(51) Int. Cl. A61K 39/395; C12Q 1/26; C12Q 1/68; C12N 5/00; A61K 31/7088; A61K 31/713; A61P 35/00

(52) U.S. Cl. 424/133.1; 435/25; 435/6; 435/375; 424/172.1; 514/44 R; 514/44 A

(57) ABSTRACT

Disclosed herein are methods for modulating the environment of a tumor, by inhibiting the activity of the extracellular enzyme lysyl oxidase-like 2 (LOXL2). The methods disclosed herein are effective in reducing tumor growth, reducing recruitment of cells to the tumor, reducing fibroblast activation, reducing desmoplasia, reducing vasculogenesis, reducing the number of TAFs, reducing growth factor production, inhibiting collagen deposition, and increasing necrosis and pyknosis in the tumor. Exemplary inhibitors of LOXL2 activity are antibodies and siRNAs.

### CROSS REFERENCE TO RELATED APPLICATIONS

This application claims the benefit of U.S. provisional patent application No. 61/235,852, filed Aug. 21, 2009, the disclosure of which is hereby incorporated by reference in its entirety for all purposes.

This application is related to U.S. provisional patent application No. 61/235,846 (filed Aug. 21, 2009) and to U.S. provisional patent application No. 61/235,796 (filed Aug. 21, 2009), the disclosures of which are hereby incorporated by reference in their entireties for all purposes.

This application is also related to co-owned United States patent application entitled “In vivo Screening Assays,” Attorney Docket No. ARBS-012, Client Ref. No. A12-US1; and to co-owned United States patent application entitled “In vitro Screening Assays,” Attorney Docket No. ARBS-013, Client Ref. No. A13-US1; each of which is filed even date herewith; and the disclosures of which are incorporated by reference in their entireties for all purposes.

### STATEMENT REGARDING FEDERAL SUPPORT

Not applicable.

### FIELD

The present application is in the fields of cancer, oncology and fibrotic diseases.

### BACKGROUND

Extensive clinical evidence and mouse models of tumorigenesis support the critical role of the microenvironment in promoting tumor growth and metastasis. The recruitment and activation of fibroblasts, vascular cells and inflammatory cells by tumor cells has been shown to facilitate metastatic potential and can impact the outcome of therapy. Epithelial malignancies of the pancreas, breast, prostate, colon, lung and uterus often contain a desmoplastic stroma composed of tumor-associated fibroblasts (TAFs) and accumulated extracellular matrix, which has been associated with a poorer prognosis. These TAFs are thought to contribute to tumorigenesis in part by stimulation of tumor angiogenesis. TAFs exhibit the smooth muscle-like contractile properties of myofibroblasts, which play a significant role in the pathologic remodeling of organs leading to fibrosis. Providing further evidence of the role that factors which modify the microenvironment play in disease progression, recent studies have shown that changes in mechanical tension of the extracellular matrix can lead to significant changes in cell morphology, activation of signaling pathways, tissue remodeling, and pathogenesis. These findings underscore the potential for new therapeutic strategies in oncology and fibrosis, targeting proteins that regulate the composition and mechanical properties of the extracellular matrix.

Lysyl oxidase-type enzymes (LOX/Ls) comprise a family of 5 enzymes sharing a conserved C-terminal enzymatic domain with divergent N-termini. LOX/Ls are copper-containing enzymes that catalyze the oxidative deamination of the epsilon-amine group in particular lysine residues to promote the covalent cross-linking of proteins such as fibrillar collagen I, a major component of desmoplastic stroma. There is some evidence that certain LOX/Ls play a role in initiation and progression of both oncologic and fibrotic diseases, and lysyl oxidase (LOX) has been shown to play a role in the development of metastasis and metastatic niche formation. See, for example, co-owned United States Patent Application Publication No. US 2009/0104201 (Apr. 23, 2009), entitled “Methods and compositions for treatment and diagnosis of fibrosis, tumor invasion, angiogenesis & metastasis,” the disclosure of which is incorporated by reference in its entirety for the purposes of describing various aspects of the biology of the lysyl oxidase-type enzymes.

Lysyl-oxidase like 2 (LOXL2) mRNA is highly expressed in a number of different solid tumors and tumor cell lines. LOXL2 has been reported to enhance the in vivo accumulation and deposition of collagen in breast tumors and gliomas formed by LOXL2-expressing cancer cells. Expression of LOXL2 protein has been described previously in breast and esophageal tumors, and squamous carcinomas, primarily with an intracellular localization, while a recent report supports a role for secreted LOXL2 in promoting tumor cell invasion in stomach cancer. Increased LOXL2 levels have also been associated with degenerative and fibrotic diseases, for example, in hepatocytes from patients with Wilson's disease or primary biliary cirrhosis and in renal tubulointerstitial fibrosis.

### SUMMARY

In the present disclosure, the inventors have identified roles for LOXL2 in (1) creation of the tumor microenvironment and (2) fibroblast activation.

Accordingly, the present disclosure provides methods and compositions for reducing desmoplasia and fibroblast activation in tumors and fibrotic disease, including but not limited to the following embodiments:

1. A method for inhibiting fibroblast activation in a tumor environment, the method comprising inhibiting the activity of lysyl oxidase-like 2 (LOXL2).

2. The method of embodiment 1, wherein the fibroblast activation is mediated by transforming growth factor-beta (TGF-β) signaling.

3. The method of embodiment 1, wherein inhibition of LOXL2 activity results in disorganization of the extracellular matrix.

4. The method of embodiment 3, wherein disorganization of the extracellular matrix results in disruption of the cytoskeleton of cells in the tumor stroma.

5. The method of embodiment 1, wherein the fibroblasts are tumor-associated fibroblasts (TAFs).

6. The method of embodiment 1, wherein the fibroblasts are myofibroblasts.

7. A method for inhibiting desmoplasia in a tumor environment, the method comprising inhibiting the activity of lysyl oxidase-like 2 (LOXL2).

8. The method of embodiment 7, wherein the tumor is a metastatic tumor.

9. A method for inhibiting vasculogenesis in a tumor environment, the method comprising inhibiting the activity of lysyl oxidase-like 2 (LOXL2).

10. The method of embodiment 9, wherein vasculogenesis comprises recruitment of vascular cells or vascular cell progenitors to a tumor environment.

11. The method of embodiment 9, wherein vasculogenesis comprises vascular branching.

12. The method of embodiment 9, wherein vasculogenesis comprises increase in vessel length.

13. The method of embodiment 9, wherein vasculogenesis comprises an increase in the number of vessels.

14. A method for reducing the number of tumor-associated fibroblasts (TAFs) in a tumor stroma, the method comprising inhibiting the activity of lysyl oxidase-like 2 (LOXL2).

15. A method for inhibiting collagen deposition in a tumor environment, the method comprising inhibiting the activity of lysyl oxidase-like 2 (LOXL2).

16. A method for modulating a tumor environment, the method comprising inhibiting the activity of lysyl oxidase-like 2 (LOXL2).

17. The method of embodiment 16, wherein modulation comprises a reduction in desmoplasia.

18. The method of embodiment 16, wherein modulation comprises a reduction in the number of tumor-associated fibroblasts (TAFs).

19. The method of embodiment 16, wherein modulation comprises a reduction in the number of myofibroblasts.

20. The method of embodiment 16, wherein modulation comprises remodeling of the cytoskeleton of a cell.

21. The method of embodiment 20, wherein the cell is a tumor cell.

22. The method of embodiment 20, wherein the cell is a fibroblast.

23. The method of embodiment 20, wherein the cell is an endothelial cell.

24. The method of embodiment 16, wherein modulation comprises a reduction in tumor vasculature.

25. The method of embodiment 16, wherein modulation comprises a reduction in collagen production.

26. The method of embodiment 16, wherein modulation comprises a reduction in fibroblast activation.

27. The method of embodiment 16, wherein modulation comprises inhibition of recruitment of fibroblasts to the tumor environment.

28. The method of embodiment 16, wherein modulation comprises a reduction in expression of a gene encoding a stromal component.

29. The method of embodiment 28, wherein the stromal component is selected from the group consisting of alpha-smooth muscle actin, Type I collagen, vimentin, matrix metalloprotease 9, and fibronectin.

30. A method for modulating the production of growth factors in a tumor environment, the method comprising inhibiting the activity of lysyl oxidase-like 2 (LOXL2).

31. The method of embodiment 30, wherein the growth factor is selected from the group consisting of vascular endothelial growth factor (VEGF) and stromal cell-derived factor-1 (SDF-1).

32. A method for increasing necrosis in a tumor, the method comprising inhibiting the activity of lysyl oxidase-like 2 (LOXL2).

33. A method for increasing pyknosis in a tumor, the method comprising inhibiting the activity of lysyl oxidase-like 2 (LOXL2).

34. The method of any of embodiments 1, 7, 9, 14, 15, 16, 30, 32 or 33, wherein the activity of LOXL2 is inhibited using an anti-LOXL2 antibody.

35. The method of embodiment 34, wherein the antibody comprises heavy chain sequences as set forth in SEQ ID NO:1 and light chain sequences as set forth in SEQ ID NO:2.

36. The method of embodiment 34, wherein the antibody is a humanized antibody.

37. The method of embodiment 36, wherein the antibody comprises heavy chain sequences as set forth in SEQ ID NO:3 and light chain sequences as set forth in SEQ ID NO:4.

38. The method of any of embodiments 1, 7, 9, 14, 15, 16, 30, 32 or 33, wherein the activity of LOXL2 is inhibited using a nucleic acid.

39. The method of embodiment 38, wherein the nucleic acid is a siRNA.

40. A method for identifying an inhibitor of LOXL2, the method comprising assaying a test molecule for its ability to modulate a tumor environment.

41. The method of embodiment 40, wherein modulation comprises a reduction in desmoplasia.

42. The method of embodiment 40, wherein modulation comprises a reduction in the number of tumor-associated fibroblasts (TAFs).

43. The method of embodiment 40, wherein modulation comprises a reduction in the number of myofibroblasts.

44. The method of embodiment 40, wherein modulation comprises remodeling of the cytoskeleton of a cell.

45. The method of embodiment 44, wherein the cell is a tumor cell.

46. The method of embodiment 44, wherein the cell is a fibroblast.

47. The method of embodiment 44, wherein the cell is an endothelial cell.

48. The method of embodiment 40, wherein modulation comprises a reduction in tumor vasculature.

49. The method of embodiment 48, wherein reduction in tumor vasculature is evidenced by reduction in the levels of CD31 and/or vascular endothelial growth factor (VEGF).

50. The method of embodiment 40, wherein modulation comprises a reduction in collagen production and/or a reduction in degree of collagen crosslinking.

51. The method of embodiment 40, wherein modulation comprises a reduction in fibroblast activation.

52. The method of embodiment 40, wherein modulation comprises inhibition of recruitment of fibroblasts to the tumor environment.

53. The method of embodiment 40, wherein modulation comprises a reduction in expression of a gene encoding a stromal component.

54. The method of embodiment 53, wherein the stromal component is selected from the group consisting of alpha-smooth muscle actin, Type I collagen, vimentin, matrix metalloprotease 9, and fibronectin.

55. The method of embodiment 40, wherein modulation comprises reduction in the levels of stromal cell-derived factor-1 (SDF-1) in the tumor environment.

56. The method of embodiment 40, wherein modulation comprises an increase in the incidence of necrosis and/or pyknosis in cells of the tumor.

57. The method of embodiment 40, wherein the test molecule is a small organic molecule with a molecular weight less than 1 kD.

58. The method of embodiment 40, wherein the test molecule is a polypeptide.

59. The method of embodiment 58, wherein the polypeptide is an antibody.

60. The method of embodiment 40, wherein the test molecule is a nucleic acid.

61. The method of embodiment 60, wherein the nucleic acid is a siRNA.

62. An inhibitor of LOXL2 for use in inhibiting fibroblast activation in a tumor environment.

63. An inhibitor of LOXL2 for use in inhibiting desmoplasia in a tumor environment.

64. An inhibitor of LOXL2 for use in inhibiting vasculogenesis in a tumor environment.

65. An inhibitor of LOXL2 for use in reducing the number of tumor-associated fibroblasts (TAFs) in a tumor stroma.

66. An inhibitor of LOXL2 for use in inhibiting collagen deposition in a tumor environment.

67. An inhibitor of LOXL2 for use in modulating a tumor environment.

68. An inhibitor of LOXL2 for use in modulating the production of growth factors in a tumor environment.

69. An inhibitor of LOXL2 for use in increasing necrosis in a tumor.

70. An inhibitor of LOXL2 for use in increasing pyknosis in a tumor.

71. The inhibitor of any of claims 62-70, wherein the inhibitor of LOXL2 is an anti-LOXL2 antibody.

72. The inhibitor of embodiment 71, wherein the antibody comprises heavy chain sequences as set forth in SEQ ID NO:1 and light chain sequences as set forth in SEQ ID NO:2.

73. The inhibitor of embodiment 71, wherein the antibody is a humanized antibody.

74. The inhibitor of embodiment 73, wherein the antibody comprises heavy chain sequences as set forth in SEQ ID NO:3 and light chain sequences as set forth in SEQ ID NO:4.

75. The inhibitor of any of embodiments 62-70, wherein the inhibitor is a nucleic acid.

76. The inhibitor of embodiment 75, wherein the nucleic acid is a siRNA.

77. A pharmaceutical composition for use in inhibiting fibroblast activation in a tumor environment, wherein the composition comprises an inhibitor of LOXL2 and a pharmaceutically acceptable excipient.

78. A pharmaceutical composition for use in inhibiting desmoplasia in a tumor environment, wherein the composition comprises an inhibitor of LOXL2 and a pharmaceutically acceptable excipient.

79. A pharmaceutical composition for use in inhibiting vasculogenesis in a tumor environment, wherein the composition comprises an inhibitor of LOXL2 and a pharmaceutically acceptable excipient.

80. A pharmaceutical composition for use in reducing the number of tumor-associated fibroblasts (TAFs) in a tumor stroma, wherein the composition comprises an inhibitor of LOXL2 and a pharmaceutically acceptable excipient.

81. A pharmaceutical composition for use in inhibiting collagen deposition in a tumor environment, wherein the composition comprises an inhibitor of LOXL2 and a pharmaceutically acceptable excipient.

82. A pharmaceutical composition for use in modulating a tumor environment, wherein the composition comprises an inhibitor of LOXL2 and a pharmaceutically acceptable excipient.

83. A pharmaceutical composition for use in modulating the production of growth factors in a tumor environment, wherein the composition comprises an inhibitor of LOXL2 and a pharmaceutically acceptable excipient.

84. A pharmaceutical composition for use in increasing necrosis in a tumor, wherein the composition comprises an inhibitor of LOXL2 and a pharmaceutically acceptable excipient.

85. A pharmaceutical composition for use in increasing pyknosis in a tumor, wherein the composition comprises an inhibitor of LOXL2 and a pharmaceutically acceptable excipient.

86. The composition of any of embodiments 77-85, wherein the inhibitor of LOXL2 is an anti-LOXL2 antibody.

87. The composition of embodiment 86, wherein the antibody comprises heavy chain sequences as set forth in SEQ ID NO:1 and light chain sequences as set forth in SEQ ID NO:2.

88. The composition of embodiment 86, wherein the antibody is a humanized antibody.

89. The composition of embodiment 88, wherein the antibody comprises heavy chain sequences as set forth in SEQ ID NO:3 and light chain sequences as set forth in SEQ ID NO:4.

90. The composition of any of embodiments 77-85, wherein the inhibitor is a nucleic acid.

91. The composition of embodiment 90, wherein the nucleic acid is a siRNA.

### BRIEF DESCRIPTION OF THE DRAWINGS

FIG. 1, Panels a-p shows that LOXL2 is highly expressed and secreted in solid tumors and in liver fibrosis. FIG. 1, Panel a shows qRT-PCR analysis of LOXL2 transcripts in solid tumors as compared to non-neoplastic tissues. FIGS. 1, Panel b and 1, Panel c show immunohistochemistry (IHC) of laryngeal squamous cell carcinoma for collagen I (FIG. 1b) and LOXL2 (FIG. 1c) expression in matched tumor sections. FIGS. 1, Panel d and 1, Panel e show IHC analysis of sections from a lung squamous cell carcinoma (grade 2) testing for expression of collagen I (FIG. 1, Panel d) and LOXL2 (FIG. 1, Panel e). FIGS. 1, Panel f and 1, Panel g show IHC analysis of LOXL2 expression in sections from a pancreatic adenocarcinoma (grade 3). FIG. 1, Panel f shows LOXL2 expression in the matrix and the tumor-stroma boundary; while LOXL2 expression on glomeruloid structures was also apparent in FIG. 1, Panel f and FIG. 1, Panel g. FIGS. 1, Panel h and 1, Panel i show IHC analysis of LOXL2 expression in an omental metastasis of an ovarian carcinoma. FIG. 1, Panel h shows tumor cell expression, and FIG. 1, Panel i shows LOXL2 expression in glomeruloid structures. FIGS. 1, Panel j and 1, Panel k show IHC of sections from a pancreatic adenocarcinoma. FIG. 1, Panel j shows LOXL2 expression, and FIG. 1, Panel k shows LOX expression. FIG. 1, Panel l shows LOXL2 expression in a section from a renal cell clear cell carcinoma. FIGS. 1, Panel m and 1n show IHC for LOXL2 expression in active Hepatitis C-induced liver fibrosis (FIG. 1, Panel m: 5× magnification; FIG. 1, Panel n: 40× magnification). FIGS. 1, Panel o and 1, Panel p shows IHC for LOXL2 and LOX expression, respectively, in sections from a steatohepatitic liver (40× magnification).

FIG. 2, Panels a-f shows that secreted LOXL2 promotes invasion of tumor cells in vitro. FIG. 2, Panels a and b show immunoflorescence analysis of cultures of Hs578t tumor cells co-stained for LOXL2 (FIG. 2, Panel a) and collagen I (FIG. 2, Panel b). Expression of collagen I and LOXL2 is co-localized in the extracellular matrix in these cultures. FIG. 2, Panels c-f show rhodamine-phalloidin staining of cultures of MCF-7 cells, after treatment of the cultured MCF-7 cells with: MCF7 conditioned medium (FIG. 2, Panel c), MDA-MB231 conditioned medium (FIG. 2, Panel d), MDA-MB231 conditioned medium that was pre-incubated with 4 ug anti-IgG antibody (FIG. 2, Panel e), or MDA-MB231 conditioned medium that was pre-incubated with 4 ug of anti-LOXL2 antibody AB0023 (FIG. 2, Panel f).

FIG. 3, Panels a-k show that LOXL2 promotes fibroblast activation in vitro and in vivo. FIG. 3, Panel a shows a protein (“Western”) blot analysis, testing for effects of tension on the expression level of LOXL2 in human foreskin fibroblasts (HFFs). Cells were grown on a tissue culture plate (lanes labeled 1), a 0.2% bis-acrylamide cross-linked collagen coated gel (lanes labeled 2), or a 0.8% bis-acrylamide cross-linked collagen coated gel (lanes labeled 3). FIGS. 3, Panel b and 3, Panel c show photographs of HFF cells transfected with a non-targeting siRNA (FIG. 3, Panel b) or a LOXL2 siRNA (FIG. 3, Panel c), and stained for collagen I at 10 days post transfection. FIG. 3, Panels d and e show photographs of HFF cells transfected with a non-targeting siRNA (FIG. 3, Panel d) or a LOXL2 siRNA (FIG. 3, Panel e), and stained with rhodamine phalloidin at 10 days post transfection. FIG. 3, Panels f and g show photographs of HFF cells grown under low tension (FIG. 3f, Panel) or high tension (FIG. 3, Panel g), then stained with rhodamine-phalloidin. FIG. 3, Panel h shows a protein (“Western”) blot of lysates from HFF cells from transwell cultures with MDA-MD-231 or MCF7-LOXL2 cells. FIG. 3, Panel i shows quantitation, by densitometry, of the results shown in FIG. 3, Panel h, indicating AB0023-specific effects on pSMAD2 and VEGF expression. FIG. 3, Panel j shows a comparison of the size of xenografts generated in the sub-renal capsule of nu/nu mice implanted with MCF7 cells (MCF7-control) or with MCF7 cells stably transfected with a LOXL2 expression vector (MCF7-LOXL2). FIG. 3, Panel k shows analysis of the xenografts by quantitative RT-PCR, to examine the relative induction of various stromal components in the LOXL2-expressing tumors. Mouse-specific primers were used, to distinguish stromal expression from expression in the implanted (human) cells. aSMA=alpha smooth muscle actin; COL1A1=Type I collagen; MMP9=matrix metalloprotease 9; FN1=fibronectin type 1; VIM=vimentin. Fold activation in the stroma of MCF7-LOXL2-induced tumors, compared to MCF7-induced tumors, is shown by the numeral above the bar representing each gene.

FIG. 4, Panels a-o show examples of inhibition of angiogenesis and vasculogenesis by the anti-LOXL2 antibody AB0023, in vitro and in vivo. FIG. 4, Panels a and b show rhodamine-phalloidin staining of HUVEC cells transfected with either a non-targeting siRNA (FIG. 4, Panel a) or a siRNA targeted to LOXL2 (FIG. 4, Panel b), then cultured for 10 days. FIG. 4, Panels c-i show results of in vitro tube formation assays, in which human umbilical vein endothelial cells (HUVEC) in culture were treated with increasing concentrations of AB0023, followed by staining for the endothelial marker CD31. The four panels show HUVEC cultured in the absence of antibody (FIG. 4, Panel c) or in the presence of 1 ug/ml (FIG. 4, Panel d), 10 ug/ml (FIG. 4, Panel e) or 50 ug/ml (FIG. 4, Panel f) of AB0023. Quantitation of the mean number of branching points (FIG. 4, Panel g), mean number of vessels (FIG. 4, Panel h) and mean total tubule length (FIG. 4, Panel i) was also conducted. FIG. 4, Panels j-m show effects of the anti-LOXL2 antibody AB0023 on vasculogenesis in a Matrigel™ plug assay. Balb/C mice were implanted in the flank with a Matrigel™ plug containing bFGF, then treated with either AB0023 or vehicle (PBST). Histology (H&E staining) of the plug in animals treated with vehicle only, at day 10 after implantation, showed evidence of branching and invading vasculature (FIG. 4, Panel j), which is virtually absent in the plug from AB0023-treated animals (FIG. 4, Panel k). CD31 staining of plugs from animals treated with vehicle only (FIG. 4, Panel l) and AB0023 (FIG. 4, Panel m) provided similar results; i.e., lack of vasculogenesis in plugs from AB0023-treated animals. FIG. 4, Panel n provides a quantitative analysis of the average number of vessels in plugs from vehicle-treated and AB0023-treated animals, indicating a ˜7-fold decrease of vasculogenesis in the AB0023-treated mice (p=0.0319). FIG. 4, Panel o shows quantitation of CD31-positive cells in the plugs from vehicle-treated and AB0023-treated mice, corroborating the decrease in vasculogenesis (p=0.0168).

FIG. 5, Panels a-u show that the anti-LOXL2 antibody AB0023 is effective in reducing stromal activation and inhibiting generation of a tumor environment in vivo in both primary tumors and metastatic xenograft models of cancer. For the results shown in FIG. 5, Panels a and b, approximately 10⁶ MDA-MB231 cells were injected into mice (in the left ventricle) to generate a disseminated bone metastasis model and, 28 days after injection, the tumor burden was assessed. Injected animals were treated with the anti-LOX antibody M64, the anti-LOXL2 antibody AB0023, Taxotere or a vehicle control. FIG. 5, Panel a shows the day 28 tumor cell burden in the femur (AB0023 p=0.0021, M64 p=0.5262); FIG. 5, Panel b shows the 28 day tumor cell burden in total ventral bone (AB0023 p=0.0197, M64 p=0.5153).

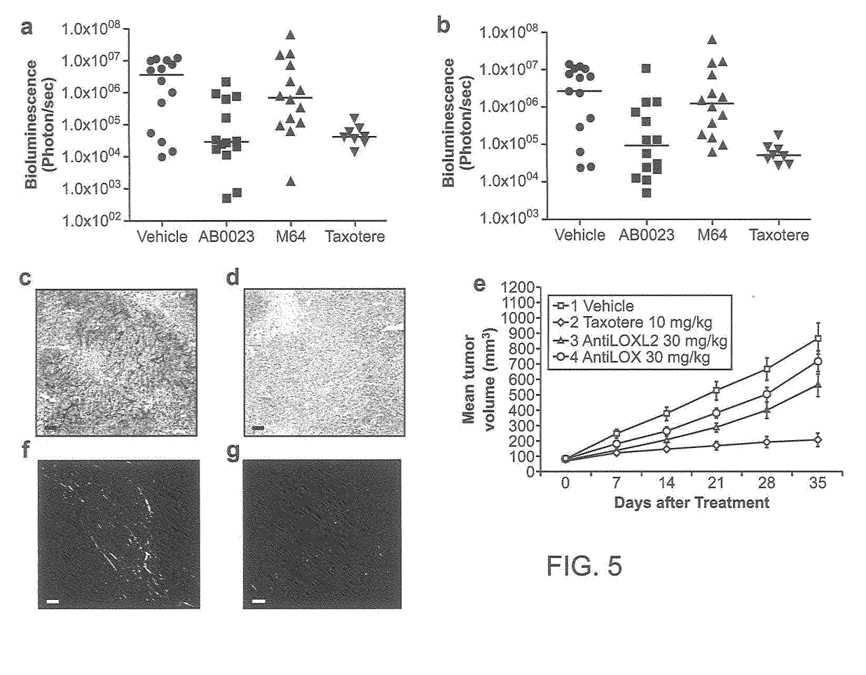

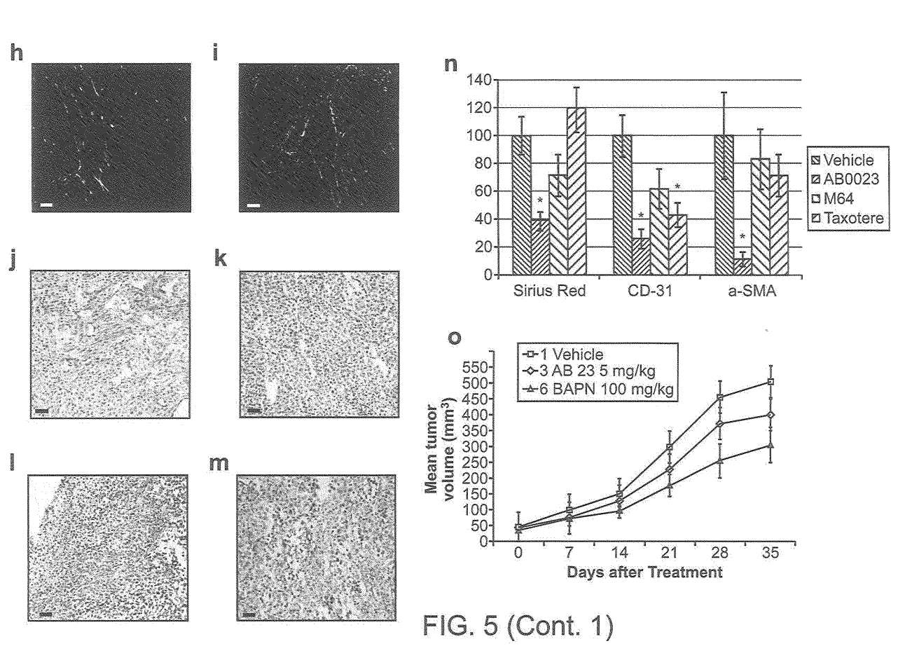

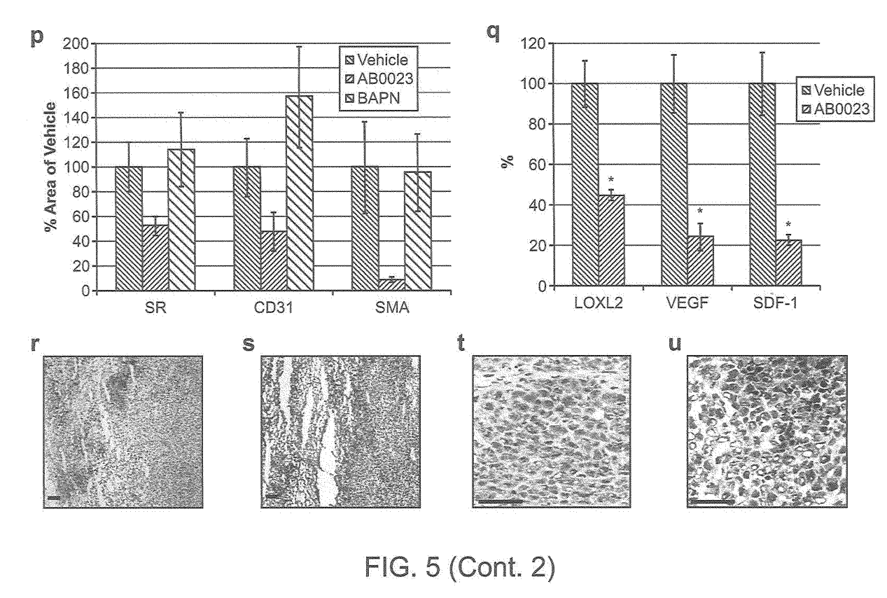

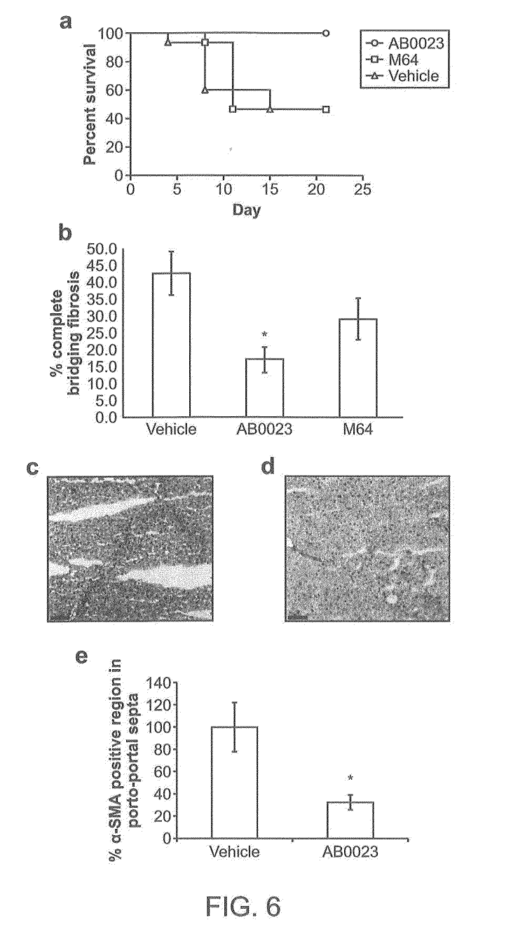

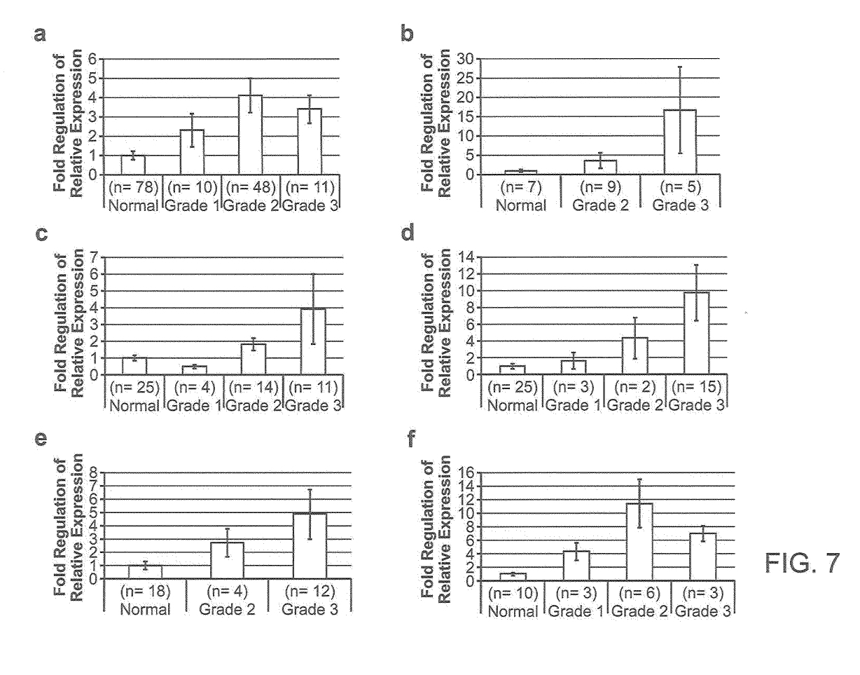

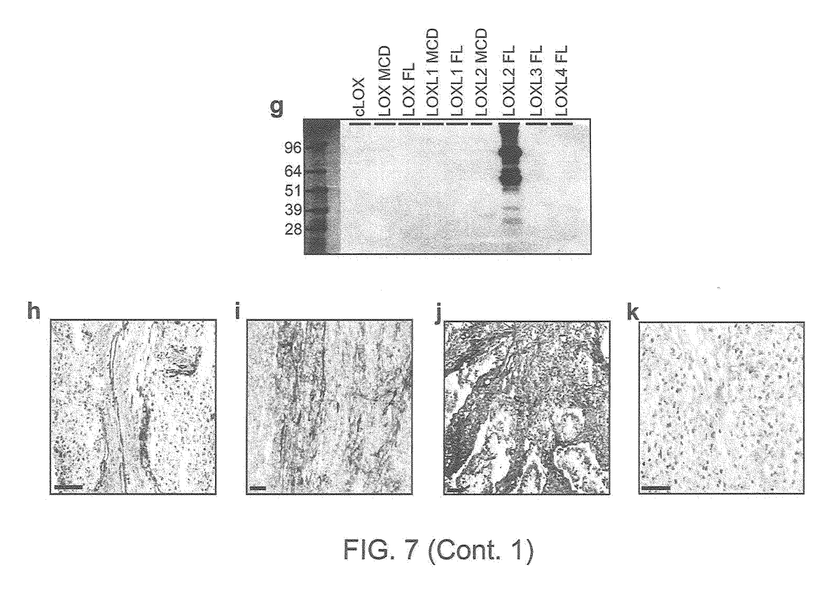

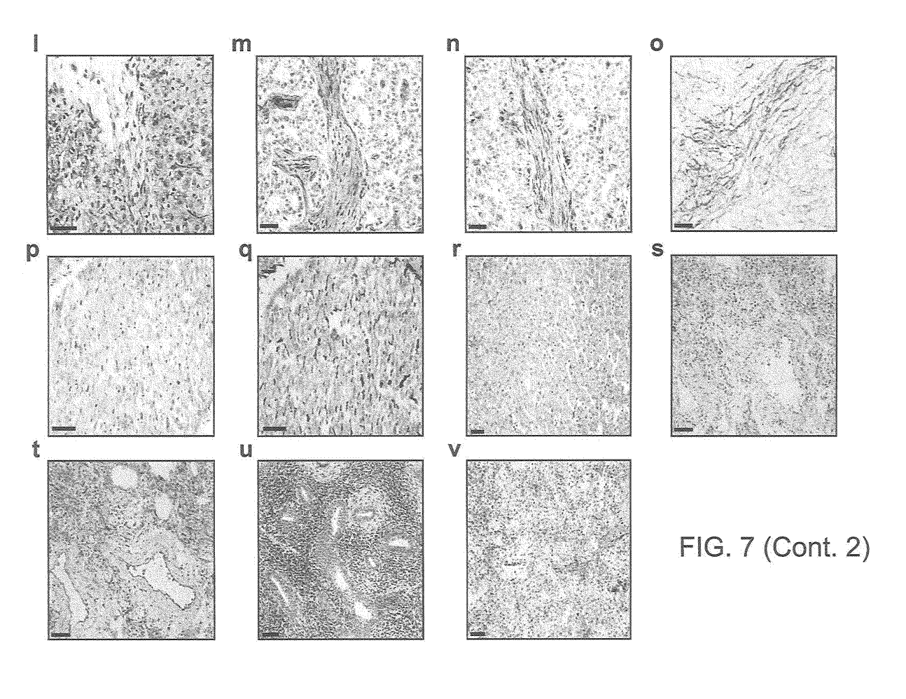

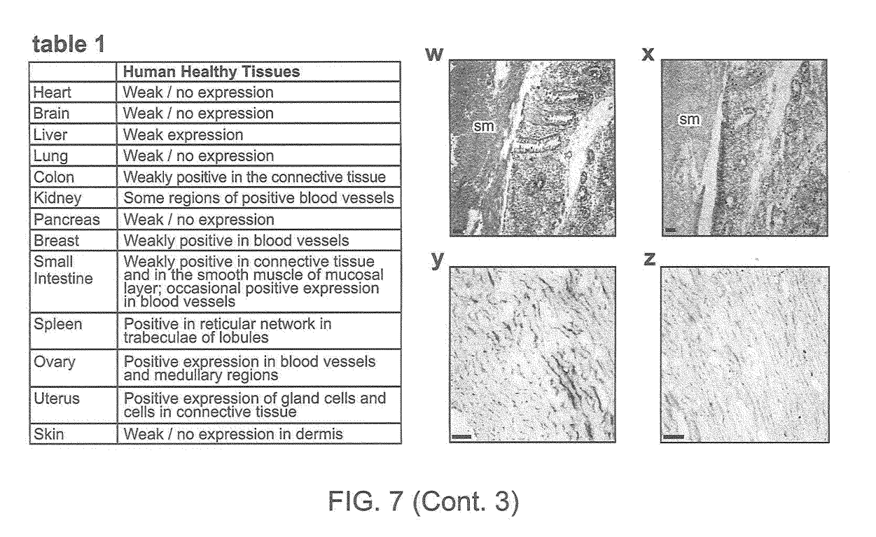

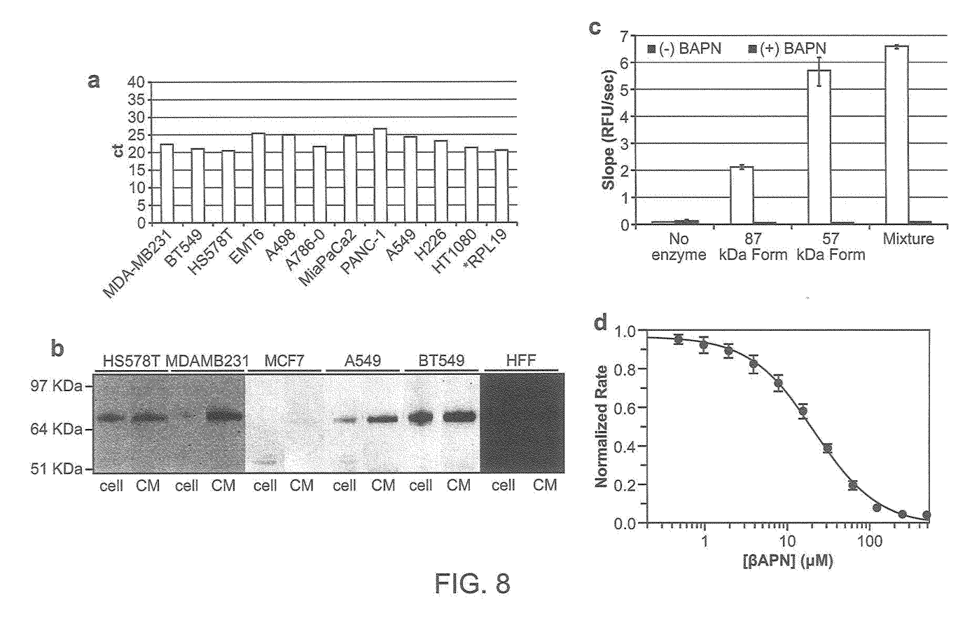

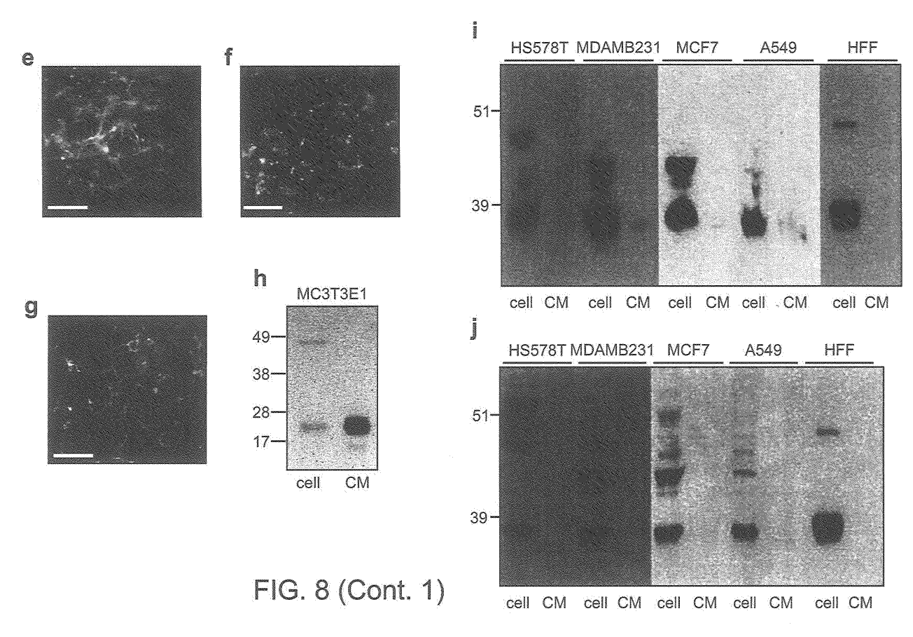

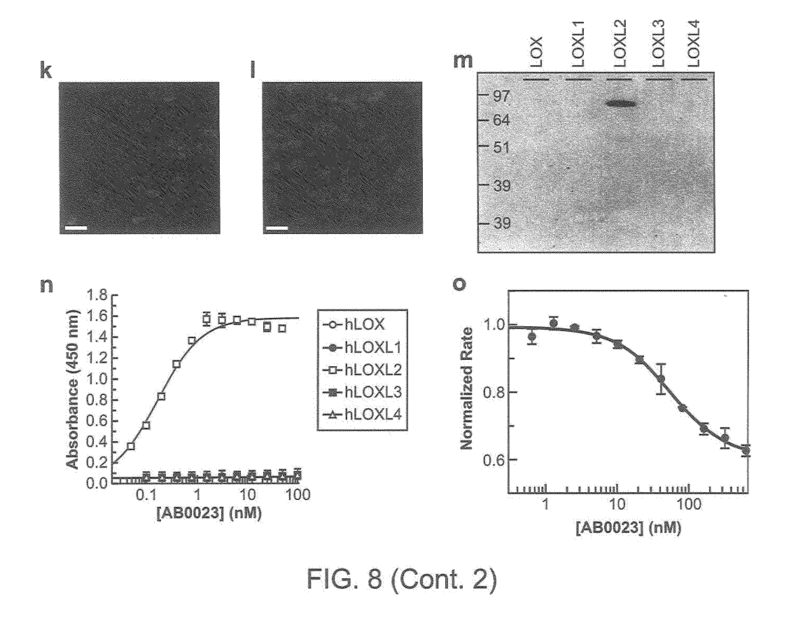

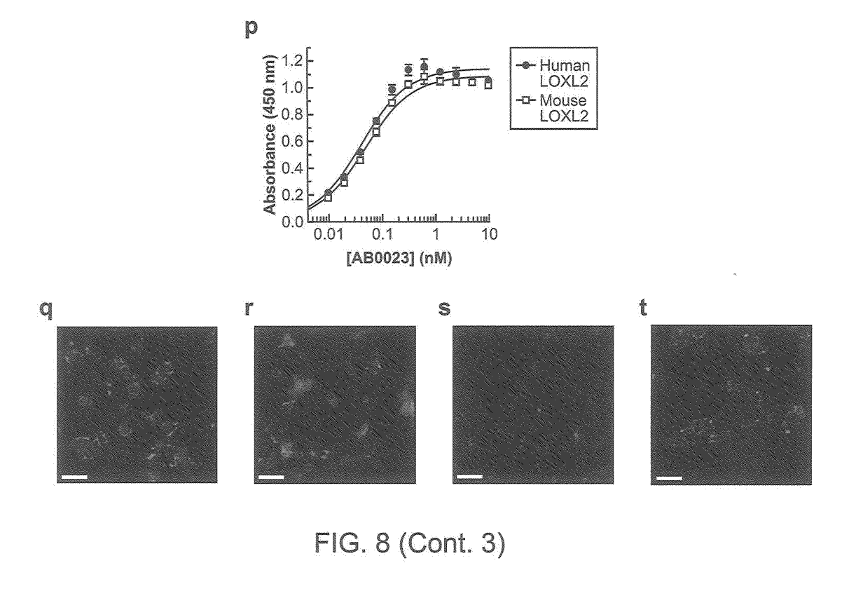

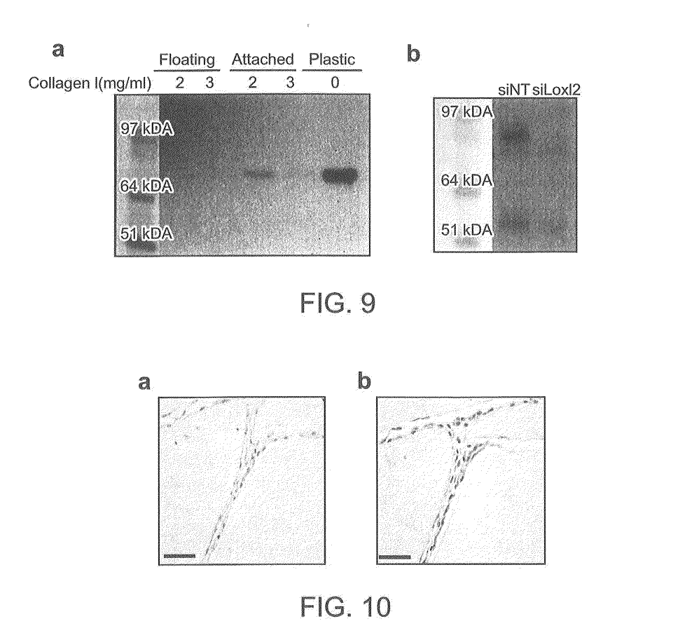

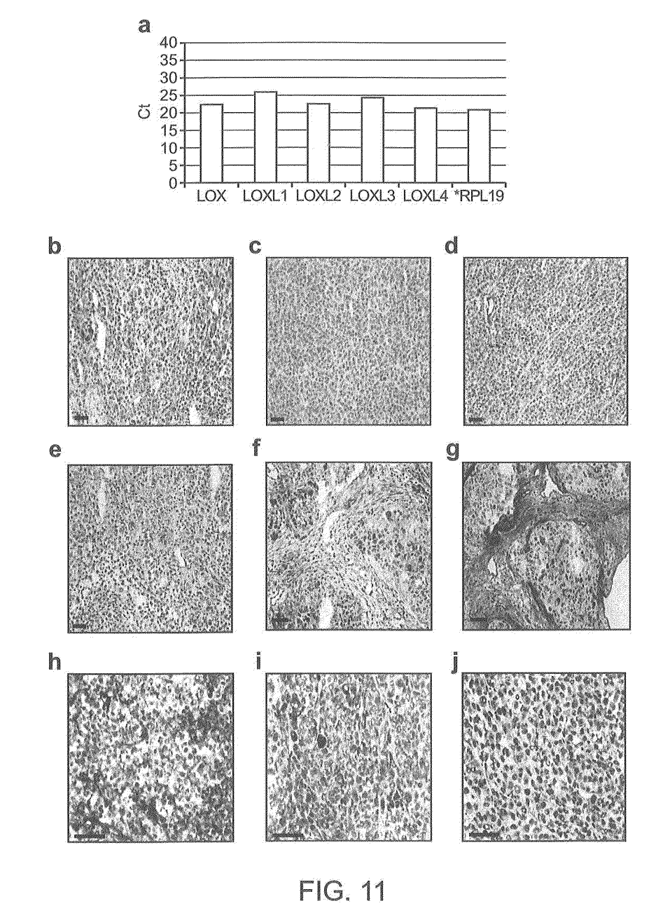

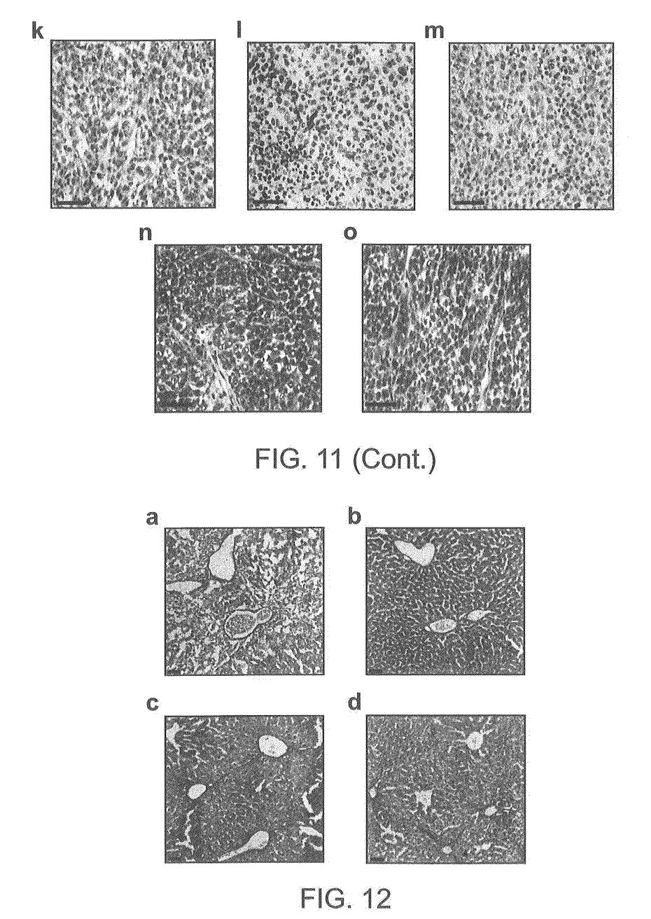

For the results shown in FIG. 5, Panels c-m, primary tumors were generated using the MDA-MB-435 cell line and treated as described. Sections from tumors generated in this model system, in which the host animals were treated only with vehicle were stained for the expression of LOXL2 (FIG. 5, Panel c) and for the expression of LOX (FIG. 5, Panel d). FIG. 5, Panel e shows measurements of tumor volumes in mice treated with vehicle only, taxotere (positive control for reduction of tumor volume), anti-LOXL2 antibody AB0023 and anti-LOX antibody M64. AB0023-treated mice maintained a significant decrease in tumor volume (45% at week 3, p=0.001; 33% at week 5, p=0.0240) while the M64 treated mice did not (27% at week 3, p=0.040; not significant at week 5). FIG. 5, Panels f-i show examples of Sirius Red staining of tumors from the vehicle-treated (FIG. 5, Panel f), AB0023-treated (FIG. 5, Panel g), M64-treated (FIG. 5, Panel h) and taxotere-treated (FIG. 5, Panel i) animals. FIG. 5, Panels j-m show IHC analyses of alpha-smooth muscle actin (α-SMA) expression in sections from tumors obtained from animals that had been treated with vehicle only (FIG. 5, Panel j), AB0023 (FIG. 5, Panel k), M64 (FIG. 5, Panel l) and taxotere (FIG. 5, Panel m). FIG. 5n shows quantitation of Sirius Red staining, α-SMA expression and CD31 expression in the tumor environment of the MDA-MB-435-induced tumors. The results indicate a 61% reduction in crosslinked collagen in the AB0023 treated mice (p=0.0027) as determined by Sirius Red staining, an 88% reduction in the presence of TAFs (p=0.011) assessed by α-SMA expression, and a 74% reduction in tumor vasculature as assessed by CD31 expression (p=0.0002).

FIG. 5, Panel o shows results of a separate study of tumor volume in MDA-MB-435-induced primary tumors in AB0023- and BAPN-treated mice; indicating a statistically significant reduction in tumor volume following treatment with the anti-LOXL2 antibody. FIG. 5, Panel p presents a quantitative analysis of Sirius Red staining (collagen production), CD-31 expression (vasculogenesis), and α-SMA expression (fibroblast activation) in MDA-MB-435-induced tumors from AB0023- and BAPN-treated mice; showing a reduction in all three markers in AB0023-treated mice. FIG. 5, Panel q shows analysis of expression of LOXL2, VEGF and SDF-1 in MDA-MB-435-induced tumors from AB0023-treated and control (vehicle-treated) mice; showing 76% reduction of VEGF levels (p=0.0001), 80% reduction of SDF1 levels (p=0.0200), and 55% reduction in LOXL2 levels (p=0.0005) in AB0023-treated MDA-MB-435 tumors.

FIG. 5, Panels r and s provide evidence of necrosis in AB0023-treated MDA-MB-435 tumors. FIG. 5, Panel r shows IHC analysis for Tumor Necrosis Factor alpha (TNF-α) in a section from an AB0023-treated MDA-MB-435 tumor. FIG. 5, Panel s shows hematoxylin and eosin (H&E) staining of a section from an AB0023-treated MDA-MB-435 tumor. FIGS. 5t and 5u provide evidence for pyknosis in AB0023-treated MDA-MB-435 tumors. While nuclei in sections of vehicle-treated tumors were well defined (FIG. 5, Panel t), those in sections of AB0023-treated tumor appeared pyknotic (FIG. 5, Panel u).

FIG. 6, Panels a-e show AB0023-mediated inhibition of CCl₄-induced liver fibrosis and myofibroblast activation. FIG. 6, Panel a shows a Kaplan Meier survival analysis of CCl₄-treated mice also treated with anti-LOXL2 antibody AB0023, anti-LOX antibody M64 or vehicle. A significant increase in survival was apparent in the AB0023 treatment arm (p=0.0029 in log rank test, or p=0.0064 in the Mantel-Cox test). FIG. 6, Panel b shows a significant decrease in the amount of bridging fibrosis in the livers of AB0023 treated mice (p=0.0020). FIG. 6, Panels c and d show IHC analysis for α-SMA in sections of the porto-portal region of a liver from a vehicle treated mouse (FIG. 6, Panel c), compared to a liver from an AB0023 treated mouse (FIG. 6, Panel d). FIG. 6, Panel e provides a quantitative analysis of α-SMA signal, demonstrating that lack of bridging fibrosis in the livers of AB0023-treated animals was accompanied by a significant reduction in the number of alpha-SMA positive myofibroblasts (p=0.0260).

FIG. 7, Panels a-z shows evidence of LOXL2 expression in various human tumors and normal tissues. (Panels a-f) Quantitative RT-PCR analysis of LOXL2 transcripts was performed on human colon adenocarcinoma (Panel a), pancreatic adenocarcinoma (Panel b), uterine adenocarcinoma (Panel c), renal cell carcinoma (Panel d), stomach adenocarcinoma (Panel e), and laryngeal squamous cell carcinoma (Panel f), a trend for increased LOXL2 transcript with increasing tumor grade was observed. (Panels g-y) A Western blot analysis of various LOX/L species shows the polyclonal antibody used for IHC of human and mouse tissue sections is specific for LOXL2 (Panel g, cLOX=mature LOX, propeptide cleaved; MCD=catalytic domain of protein only; FL=full length protein; this specificity was also confirmed by ELISA (data not shown)). Additional examples of LOXL2 expression in: breast infiltrative ductal carcinoma (Panel h), uterine endometrial carcinoma (Panel i), colon adenocarcinoma (Panel j), hepatocellular carcinoma (Panel k, also stained for LOX expression (Panel l)), neurendocrine carcinoma of the pancreas (Panel m, also stained for LOX expression (Panel n)), melanoma (Panel o), normal heart (Panel p, also stained with CD31 (Panel q)), normal liver (Panel r), normal lung (Panel s, also stained with CD31 (Panel t)), normal ovary (Panel u), normal spleen Panel v), normal smooth muscle (Panel x, also stained for LOX expression (Panel w)), and normal artery (z, also stained for LOX expression (Panel y)). Table 1 presented in FIG. 7 summarizes LOXL2 expression in human healthy tissues. Human normal tissues were stained with the anti-LOXL2 polyclonal antibody and a qualitative assessment of the relative LOXL2 expression levels was compiled.

FIG. 8, Panels a-t shows that secreted LOXL2 promotes remodeling and invasion of tumor cells in vitro (Panel a) A qRT-PCR analysis (Ct values) of LOXL2 transcripts in various tumor and fibroblast cell lines (normoxic conditions, RPL19 used for reference). (Panel b) A western analysis of LOXL2 expression in human tumor and fibroblast cell lines (whole cell pellet=cell; conditioned media=CM). (Panel c) An Amplex Red assay using purified recombinant human LOXL2 showed both the 87 kD and 55 kD forms of LOXL2 to be active and inhibited by BAPN (mixture=˜50:50 mixture of both forms). (Panel d) The dose response curve for BAPN inhibition of purified recombinant human LOXL2 (Amplex Red assay; data normalized to control). (Panels e-g) HS578t were transfected with a non-targeting siRNA (siNT) or a LOXL2 siRNA and then stained for expression of LOXL2 or collagen I. LOXL2 expression co-localized with collagen I (siNT stained for LOXL2 (Panel e), LOXL2 siRNA stained for LOXL2 (Panel f) and collagen I (Panel g)). (Panel h) Secretion of LOX in MC3T3E1 (CM Concentrated ˜20×). (Panels i,j) LOX expression in tumor or fibroblast cell lines under normoxic (Panel i) or hypoxic (Panel j) conditions showed no detectable secretion of LOX (CM concentrated ˜20×). (k,l) MDA-MB-231 cells transfected with non-targeting shRNA (Panel k) and stained with rhodamine-phalloidin retained their mesenchymal phenotype while those transfected with a LOXL2 shRNA (Panel l) adopted a more epithelial phenotype. (Panels m,n) A western blot analysis and ELISA (Panel n) both show AB0023 is specific for LOXL2. (Panel o) A dose response curve for AB0023 inhibition of LOXL2 enzymatic activity (Amplex Red assay). (Panel p) AB0023 cross reacts with mouse LOXL2. (Panels q-t) The growth media of SW620 cells was supplemented with the following conditioned medias: MDA-MB-231 CM (Panel r) or HEK293 CM transfected with an empty vector (Panel q), LOXL2 (Panel s) or LOXL2 Y689F (Panel t). The cells were stained with rhodamine-phalloidin.

FIG. 9, Panels a-b shows LOXL2 expression in HFF cells under varying tension and confirmation of LOXL2 knockdown. (Panel a) HFF cells were grown in tissue culture plates (Plastic) or collagen I gels containing 2 mg/ml (2) or 3 mg/ml (3) collagen I. The gels were either detached (Floating) or anchored to the culture dish (Attached). The conditioned media was analyzed by Western analysis and probed for LOXL2 expression. (Panel b) HFF cells were transfected with non-targeting siRNA (siNT) of LOXL2 siRNA (siLOXL2) and the conditioned media probed for LOXL2 expression via western blot analysis.

FIG. 10, Panels a-b shows LOXL2 expression in infiltrating cells in an in vivo matrigel plug (Panels a,b) IHC analysis of endothelial cell infiltrates in a matrigel plug confirms LOXL2 expression (Panel a). The section was also stained with CD31 (Panel b) to confirm presence of endothelial cells.

FIG. 11, Panels a-o shows AB0023 efficacy in vivo in primary tumor and metastatic xenograft models of cancer (Panel a) A qRT-PCR analysis of MDA-MB-231 cells confirms the transcription of all LOX/L proteins (RPL-19 used as a reference). (Panels b-e) CD31 staining of MDA-MB-435 established primary tumors harvested from mice treated with a vehicle (Panel b), anti-LOXL2 antibody AB0023 (Panel c), anti-LOX antibody M64 (Panel d), or Taxotere (Panel e) showed a 74% reduction in CD31 staining in the AB0023 treatment relative to vehicle (p=0.0002). (Panels f, g) A human breast adinocarcinoma stained for expression of VEGF (Panel f) and LOXL2 (Panel g) shows similarities in TAF expression. (Panel h-o) MDA-MB-435 established primary tumors from vehicle and AB0023 treated mice were stained for expression of LOXL2 (Panel h, vehicle treatment; Panel i, AB0023 treatment), VEGF (Panel j, vehicle; Panel k, AB0023), and SDF-1 (Panel l, vehicle; Panel m, AB0023), as well as with H&E (Panel n, vehicle, Panel o, AB0023).

FIG. 12, Panels a-d shows fibrogenesis in murine livers from a CCl4-induced fibrosis model. (Panels a-d) A murine CCl4-induced liver fibrosis model showed early evidence of liver damage and fibrosis, as evidenced by collagen I staining (Sirius Red) of a liver from an early-death animal (day 11) (Panel a) compared to a healthy liver (Panel b). Example of livers used in analysis of bridging fibrosis: the AB0023 treated mice (Panel d) had significantly less complete bridging fibrosis (p=0.002) as compared to the vehicle (Panel c).

### DETAILED DESCRIPTION

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N.J., 2000; and the series “Methods in Enzymology,” Academic Press, San Diego, Calif.

The present inventors have identified a role for matrix enzyme lysyl oxidase-like-2 (LOXL2) in the creation of the pathologic microenvironment of oncologic and fibrotic diseases. Analysis of human tumors and liver fibrosis revealed widespread and conserved expression of LOXL2 by activated fibroblasts and neovasculature. The inhibition of LOXL2 with an anti-LOXL2 monoclonal antibody was efficacious in both primary and metastatic xenograft models of cancer, as well as CCl₄-induced liver fibrosis. Inhibition of LOXL2 resulted not only in a substantial reduction in fibroblast activation, fibroblast recruitment, desmoplasia, and vascularization, but also in significantly decreased production of pro-angiogenic growth factors and cytokines such as VEGF and SDF1. Inhibition of lysyl oxidase (LOX) had little, if any such effects.

The small molecule beta-aminoproprionitrile (BAPN) has been used to explore the effects of inhibition of LOX/L activity in vitro and in vivo. BAPN covalently modifies the lysine-tyrosine quinone in the enzymatic domain and thus acts as an irreversible inhibitor. BAPN lacks specificity as it inhibits not only the potentially diverse activities of different LOX/Ls, but similar domains in other amine oxidases as well. The anti-LOXL2 antibody outperformed the small molecule pan-lysyl oxidase inhibitor beta-aminoproprionitrile (BAPN). The anti-LOXL2 antibody acts as a specific inhibitor of LOXL2, and represents a new therapeutic approach with broad applicability in oncologic and fibrotic diseases.

The present inventors have uncovered a role for LOXL2 in establishing the pathologic microenvironment of tumors and fibrotic disease, and have demonstrated it is a target for therapy. LOXL2 protein expression and secretion, by TAFs and tumor vasculature, is widespread among solid tumors, and is particularly evident at the tumor-stroma interface. LOXL2 expression is also pronounced in regions of desmoplasia and glomeruloid microvascular proliferation, both of which are associated with poor outcome in several cancers. In active liver fibrosis, LOXL2 was similarly expressed at the hepatocyte-myofibroblast interface and associated neovasculature.

The inventors have further determined that expression of LOXL2 results in remodeling of the actin cytoskeleton in multiple cells types, including tumor cells of epithelial origin, endothelial cells, and fibroblasts. One contribution of LOXL2 to disease progression is the activation and recruitment of disease-associated fibroblasts, most likely through its enzymatically-catalyzed cross-linking of fibrillar collagen and corresponding changes in local matrix tension. In tumors and in liver fibrosis, increases in tension can lead to disease-associated cellular differentiation. Beyond the production of fibrillar collagens and the creation of tension within tissue, TAFs (and potentially also myofibroblasts) secrete many of the angiogenic, vasculogenic and chemotactic growth factors and cytokines that support ongoing tumorigenesis and fibrosis.

It is disclosed herein that specific inhibition of activity of secreted LOXL2, in models of both cancer and fibrosis, resulted in significant reduction of disease as assessed by a variety of parameters. Inhibition of LOXL2 is capable of directly affecting angiogenesis, as well as invasion and differentiation of disease-associated epithelia. However, inhibition of angiogenesis alone is not completely responsible for the effects observed following inhibition of LOXL2, inasmuch as potent anti-angiogenics directed at the VEGFR and P1GF pathways do not affect the number of αSMA positive cells in tumors, as does inhibition of LOXL2.

It is also disclosed herein that inhibition of LOXL2 in vivo resulted in inhibition of fibroblast activation and recruitment, the consequences of which include substantial reduction of desmoplasia and the expression of pro-angiogenic growth factors and cyotkines, lack of formation of tumor vasculature, and increased necrosis and autophagy of tumor cells. Production of fibrillar collagen, a hallmark of fibrosis, was also greatly reduced by inhibition of LOXL2, not due to direct regulation of collagen expression but rather due to the substantial reduction in the number of activated myofibroblasts (the cell type responsible for the majority of collagen production).

Many potential sources of disease-associated activated fibroblasts have been proposed, including fibrocytes and other bone-marrow derived cells, resident fibroblasts or other precursors, and epithelial-to-mesenchymal transition (EMT) of epithelial cells. In the work disclosed herein, therapeutic benefits were obtained in three very different mouse models involving different sites of disease, and in the 2 models amenable for further analysis, the mechanism appeared conserved, wherein fibroblast activation was substantially reduced. These results suggest that LOXL2 is important for the ultimate differentiation and activation of fibroblasts, independent of their origin.

The inventors show herein that inhibition of LOXL2 alone was sufficient to obtain therapeutic efficacy, despite the use of model systems containing cells that make multiple lysyl oxidase-type enzymes, including LOX. In comparison, the use of a particular LOX-specific monoclonal antibody targeting a peptide previously identified as generating a polyclonal antiserum capable of inhibiting LOX enzymatic activity provided little therapeutic benefit in models of oncology and fibrosis.

The differential expression of LOXL2 in diseased versus healthy tissues provides a functional therapeutic window. In support of the safety of anti-LOXL2 antibody AB0023, the inventors found AB0023 to be well-tolerated at a dosage of 50 mg/kg twice per week for 14 weeks in mice, with no impact on weight or behavior and no drug-related observations upon necropsy, hematology, clinical chemistry and histopathology. Pilot studies in cynomolgus monkeys with a humanized anti-LOXL2 variant (AB0024) provided further support that anti-LOXL2 antibody therapy was well tolerated upon repeat dosing at 100 mg/kg.

Antibody therapeutics provide one example of a highly specific mechanism for inhibition. Indeed, specific targeting of secreted LOXL2 with an antibody (AB0023) that inhibits its enzymatic activity outperformed the less-specific cell-permeable pan-inhibitor BAPN, in cell based assays and in vivo. (Note that contrary to previous reports, find LOXL2 was found to be readily inhibited by BAPN in vitro, with a low nanomolar IC50, similar to that observed for LOX; FIG. 8, panel D and Rodriguez et al. (2010) J. Biol. Chem. 285:20964-20974). Apart from specificity, this therapeutic mode provides an additional advantage: as non-competitive allosteric inhibitors of LOXL2, AB0023 and AB0024 act independently of substrate concentration, or of the state of association between LOXL2 and its substrate, whereas the irreversible inhibitor BAPN behaves as a competitive inhibitor and is less effective at high substrate concentrations or under conditions where LOXL2 is bound to its substrate. This alternative mechanism of inhibition represents a novel therapeutic approach that has broad applicability for matrix enzymes functioning within a dynamic complex cellular milieu containing a local high concentration of substrate, such as fibrillar collagen, in active disease.

Allosteric inhibition of LOXL2, as described herein, represents a new approach to inhibiting the growth and progression of tumors and fibrotic diseases, by targeting fundamental shared features of disease progression, e.g., the creation of the stromal compartment or matrix microenvironment or metastatic niche. That is, inhibition of a single target (LOXL2) has multiple effects on a number of different drivers of desmoplasia, Targeting of LOXL2 can be made highly specific through use of a monoclonal antibody. In addition, targeting the genetically more stable stromal cells of the tumor microenvironment offers the potential for reduced likelihood of drug resistance.

### DEFINITIONS

“Tumor environment” refers to a tumor and its surrounding tissue. A subset of the tumor environment is the tumor-stroma interface; i.e., the periphery of the tumor (e.g., the tumor capsule) along with the adjacent stromal tissue. Another subset is the tumor itself; yet another subset is the stromal tissue outside of a tumor.

“Fibroblast activation” refers to a process by which normal fibroblasts are converted to tumor-associated fibroblasts (TAFs) in response to signals (e.g., growth factors, cytokines) released by tumor cells. One example of such a growth factor is Transforming Growth Factor-beta (TGF-β). Exemplary consequences of fibroblast activation are increased expression of alpha-smooth muscle actin (αSMA) and increased expression of vascular endothelial growth factor (VEGF) in the activated fibroblasts.

“Tumor-associated fibroblasts (TAFs)” are fibroblasts that have undergone fibroblast activation and are characterized, inter alia, by increased expression of alpha-smooth muscle actin (αSMA) and vascular endothelial growth factor (VEGF).

“Myofibroblasts” are cells with characteristics of both fibroblasts and smooth muscle cells. They can be present in fibrotic tissue and are characterized, inter alia, by expression of alpha-smooth muscle actin.

“Desmoplasia” refers to the growth of fibrous or connective tissue. Some tumors elicit a desmoplastic reaction, i.e., the pervasive growth of dense fibrous tissue around the tumor.

“Angiogenesis” refers to the formation of new blood vessels from pre-existing vessels.

“Vasculogenesis” refers to the formation of new blood vessels in the absence of pre-existing vessels.

Tumor Stroma

Growth and development of a tumor rely on interactions between the tumor and its surrounding stromal tissue. Tumors grow within a stromal framework containing connective tissue, fibroblasts, myofibroblasts, white blood cells, endothelial cells, pericytes and smooth muscle cells. The growing tumor influences the surrounding stroma by, inter alia, secreting growth factors (that influence the behavior of the stromal cells) and secreting proteases (that remodel stromal extracellular matrix). Stromal cells, in return, secrete growth factors that stimulate growth and division of the tumor cells; and secrete proteases that further modify the matrix. In this fashion, a tumor and its surrounding stromal tissue form a tumor environment that supports further growth of the tumor. For example, research has shown that certain carcinomas depend on the presence of tumor-associated fibroblasts for continued growth, and will not grow at a detectable or appreciable level in the presence of normal fibroblasts. It has also been shown that robust growth of certain tumors requires a particular matrix metalloprotease normally secreted by mast cells, which acts by releasing angiogenic factors from the extracellular matrix.

Lysyl Oxidase-Type Enzymes

As used herein, the terms “lysyl oxidase-type enzyme” and “LOX/L” refer to a member of a family of proteins that, inter alia, catalyzes oxidative deamination of ε-amino groups of lysine and hydroxylysine residues, resulting in conversion of peptidyl lysine to peptidyl-α-aminoadipic-δ-semialdehyde (allysine) and the release of stoichiometric quantities of ammonia and hydrogen peroxide:

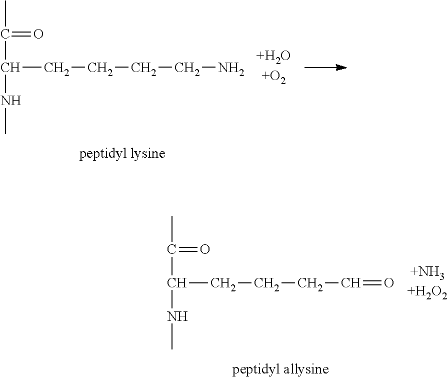

This reaction most often occurs extracellularly, on lysine residues in collagen and elastin. The aldehyde residues of allysine are reactive and can spontaneously condense with other allysine and lysine residues, resulting in crosslinking of collagen molecules to form collagen fibrils.

Lysyl oxidase-type enzymes have been purified from chicken, rat, mouse, bovines and humans. All lysyl oxidase-type enzymes contain a common catalytic domain, approximately 205 amino acids in length, located in the carboxy-terminal portion of the protein and containing the active site of the enzyme. The active site contains a copper-binding site which includes a conserved amino acid sequence containing four histidine residues which coordinate a Cu(II) atom. The active site also contains a lysyltyrosyl quinone (LTQ) cofactor, formed by intramolecular covalent linkage between a lysine and a tyrosine residue (corresponding to lys314 and tyr349 in rat lysyl oxidase, and to lys320 and tyr355 in human lysyl oxidase). The sequence surrounding the tyrosine residue that forms the LTQ cofactor is also conserved among lysyl oxidase-type enzymes. The catalytic domain also contains ten conserved cysteine residues, which participate in the formation of five disulfide bonds. The catalytic domain also includes a fibronectin binding domain. Finally, an amino acid sequence similar to a growth factor and cytokine receptor domain, containing four cysteine residues, is present in the catalytic domain. Despite the presence of these conserved regions, the different lysyl oxidase-type enzymes can be distinguished from one another, both within and outside their catalytic domains, by virtue of regions of divergent nucleotide and amino acid sequence.

The first member of this family of enzymes to be isolated and characterized was lysyl oxidase (EC 1.4.3.13); also known as protein-lysine 6-oxidase, protein-L-lysine:oxygen 6-oxidoreductase (deaminating), or LOX. See, e.g., Harris et al., Biochim. Biophys. Acta 341:332-344 (1974); Rayton et al., J. Biol. Chem. 254:621-626 (1979); Stassen, Biophys. Acta 438:49-60 (1976).

Additional lysyl oxidase-type enzymes were subsequently discovered. These proteins have been dubbed “LOX-like,” or “LOXL.” They all contain the common catalytic domain described above and have similar enzymatic activity. Currently, five different lysyl oxidase-type enzymes are known to exist in both humans and mice: LOX and the four LOX related, or LOX-like proteins LOXL1 (also denoted “lysyl oxidase-like,” “LOXL” or “LOL”), LOXL2 (also denoted “LOR-1”), LOXL3 (also denoted “LOR-2”), and LOXL4. Each of the genes encoding the five different lysyl oxidase-type enzymes resides on a different chromosome. See, for example, Molnar et al., Biochim Biophys Acta. 1647:220-24 (2003); Csiszar, Prog. Nucl. Acid Res. 70:1-32 (2001); WO 01/83702 published on Nov. 8, 2001, and U.S. Pat. No. 6,300,092, all of which are incorporated by reference herein. A LOX-like protein termed LOXC, with some similarity to LOXL4 but with a different expression pattern, has been isolated from a murine EC cell line. Ito et al. (2001) J. Biol. Chem. 276:24023-24029. Two lysyl oxidase-type enzymes, DmLOXL-1 and DmLOXL-2, have been isolated from Drosophila.

Although all lysyl oxidase-type enzymes share a common catalytic domain, they also differ from one another, particularly in their amino-terminal regions. The four LOXL proteins have amino-terminal extensions, compared to LOX. Thus, while human preproLOX (i.e., the primary translation product prior to signal sequence cleavage, see below) contains 417 amino acid residues; LOXL1 contains 574, LOXL2 contains 638, LOXL3 contains 753 and LOXL4 contains 756.

Within their amino-terminal regions, LOXL2, LOXL3 and LOXL4 contain four repeats of the scavenger receptor cysteine-rich (SRCR) domain. These domains are not present in LOX or LOXL1. SRCR domains are found in secreted, transmembrane, or extracellular matrix proteins, and are known to mediate ligand binding in a number of secreted and receptor proteins. Hoheneste et al. (1999) Nat. Struct. Biol. 6:228-232; Sasaki et al. (1998) EMBO J. 17:1606-1613. In addition to its SRCR domains, LOXL3 contains a nuclear localization signal in its amino-terminal region. A proline-rich domain appears to be unique to LOXL1. Molnar et al. (2003) Biochim. Biophys. Acta 1647:220-224. The various lysyl oxidase-type enzymes also differ in their glycosylation patterns.

Tissue distribution also differs among the lysyl oxidase-type enzymes. Human LOX mRNA is highly expressed in the heart, placenta, testis, lung, kidney and uterus, but marginally in the brain and liver. mRNA for human LOXL1 is expressed in the placenta, kidney, muscle, heart, lung, and pancreas and, similar to LOX, is expressed at much lower levels in the brain and liver. Kim et al. (1995) J. Biol. Chem. 270:7176-7182. High levels of LOXL2 mRNA are expressed in the uterus, placenta, and other organs, but as with LOX and LOXL1, low levels are expressed in the brain and liver. Jourdan Le-Saux et al. (1999) J. Biol. Chem. 274:12939:12944. LOXL3 mRNA is highly expressed in the testis, spleen, and prostate, moderately expressed in placenta, and not expressed in the liver, whereas high levels of LOXL4 mRNA are observed in the liver. Huang et al. (2001) Matrix Biol. 20:153-157; Maki and Kivirikko (2001) Biochem. J. 355:381-387; Jourdan Le-Saux et al. (2001) Genomics 74:211-218; Asuncion et al. (2001) Matrix Biol. 20:487-491.

The expression and/or involvement of the different lysyl oxidase-type enzymes in diseases also varies. See, for example, Kagen (1994) Pathol. Res. Pract. 190:910-919; Murawaki et al. (1991) Hepatology 14:1167-1173; Siegel et al. (1978) Proc. Natl. Acad. Sci. USA 75:2945-2949; Jourdan Le-Saux et al. (1994) Biochem. Biophys. Res. Comm. 199:587-592; and Kim et al. (1999) J. Cell Biochem. 72:181-188. Lysyl oxidase-type enzymes have also been implicated in a number of cancers, including head and neck cancer, bladder cancer, colon cancer, esophageal cancer and breast cancer. See, for example, Wu et al. (2007) Cancer Res. 67:4123-4129; Gorough et al. (2007) J. Pathol. 212:74-82; Csiszar (2001) Prog. Nucl. Acid Res. 70:1-32 and Kirschmann et al. (2002) Cancer Res. 62:4478-4483.

Thus, although the lysyl oxidase-type enzymes exhibit some overlap in structure and function, each has distinct structure and functions as well. With respect to structure, for example, certain antibodies raised against the catalytic domain of the human LOX protein do not bind to human LOXL2. With respect to function, it has been reported that targeted deletion of LOX appears to be lethal at parturition in mice, whereas LOXL1 deficiency causes no severe developmental phenotype. Hornstra et al. (2003) J. Biol. Chem. 278:14387-14393; Bronson et al. (2005) Neurosci. Lett. 390:118-122.

Although the most widely documented activity of lysyl oxidase-type enzymes is the oxidation of specific lysine residues in collagen and elastin outside of the cell, there is evidence that lysyl oxidase-type enzymes also participate in a number of intracellular processes. For example, there are reports that some lysyl oxidase-type enzymes regulate gene expression. Li et al. (1997) Proc. Natl. Acad. Sci. USA 94:12817-12822; Giampuzzi et al. (2000) J. Biol. Chem. 275:36341-36349. In addition, LOX has been reported to oxidize lysine residues in histone H1. Additional extracellular activities of LOX include the induction of chemotaxis of monocytes, fibroblasts and smooth muscle cells. Lazarus et al. (1995) Matrix Biol. 14:727-731; Nelson et al. (1988) Proc. Soc. Exp. Biol. Med. 188:346-352. Expression of LOX itself is induced by a number of growth factors and steroids such as TGF-β, TNF-α and interferon. Csiszar (2001) Prog. Nucl. Acid Res. 70:1-32. Recent studies have attributed other roles to LOX in diverse biological functions such as developmental regulation, tumor suppression, cell motility, and cellular senescence.

Examples of lysyl oxidase (LOX) proteins from various sources include enzymes having an amino acid sequence substantially identical to a polypeptide expressed or translated from one of the following sequences: EMBL/GenBank accessions: M94054; AAA59525.1—mRNA; S45875; AAB23549.1—mRNA; S78694; AAB21243.1—mRNA; AF039291; AAD02130.1—mRNA; BC074820; AAH74820.1—mRNA; BC074872; AAH74872.1—mRNA; M84150; AAA59541.1—Genomic DNA. One embodiment of LOX is human lysyl oxidase (hLOX) preproprotein.

Exemplary disclosures of sequences encoding lysyl oxidase-like enzymes are as follows: LOXL1 is encoded by mRNA deposited at GenBank/EMBL BC015090; AAH15090.1; LOXL2 is encoded by mRNA deposited at GenBank/EMBL U89942; LOXL3 is encoded by mRNA deposited at GenBank/EMBL AF282619; AAK51671.1; and LOXL4 is encoded by mRNA deposited at GenBank/EMBL AF338441; AAK71934.1.

The primary translation product of the LOX protein, known as the prepropeptide, contains a signal sequence extending from amino acids 1-21. This signal sequence is released intracellularly by cleavage between Cys21 and Ala22, in both mouse and human LOX, to generate a 46-48 kDa propeptide form of LOX, also referred to herein as the full-length form. The propeptide is N-glycosylated during passage through the Golgi apparatus to yield a 50 kDa protein, then secreted into the extracellular environment. At this stage, the protein is catalytically inactive. A further cleavage, between Gly168 and Asp169 in mouse LOX, and between Gly174 and Asp175 in human LOX, generates the mature, catalytically active, 30-32 kDA enzyme, releasing a 18 kDa propeptide. This final cleavage event is catalyzed by the metalloendoprotease procollagen C-proteinase, also known as bone morphogenetic protein-1 (BMP-1). Interestingly, this enzyme also functions in the processing of LOX's substrate, collagen. The N-glycosyl units are subsequently removed.

Potential signal peptide cleavage sites have been predicted at the amino termini of LOXL1, LOXL2, LOXL3, and LOXL4. The predicted signal cleavage sites are between Gly25 and Gln26 for LOXL1, between Ala25 and Gln26, for LOXL2, between Gly25 and Ser26 for LOXL3 and between Arg23 and Pro24 for LOXL4.

A BMP-1 cleavage site in the LOXL1 protein has been identified between Ser354 and Asp355. Borel et al. (2001) J. Biol. Chem. 276:48944-48949. Potential BMP-1 cleavage sites in other lysyl oxidase-type enzymes have been predicted, based on the consensus sequence for BMP-1 cleavage in procollagens and pro-LOX being at an Ala/Gly-Asp sequence, often followed by an acidic or charged residue. A predicted BMP-1 cleavage site in LOXL3 is located between Gly447 and Asp448; processing at this site may yield a mature peptide of similar size to mature LOX. A potential cleavage site for BMP-1 was also identified within LOXL4, between residues Ala569 and Asp570. Kim et al. (2003) J. Biol. Chem. 278:52071-52074. LOXL2 may also be proteolytically cleaved analogously to the other members of the LOXL family and secreted. Akiri et al. (2003) Cancer Res. 63:1657-1666.

As expected from the existence of a common catalytic domain in the lysyl oxidase-type enzymes, the sequence of the C-terminal 30 kDa region of the proenzyme in which the active site is located is highly conserved (approximately 95%). A more moderate degree of conservation (approximately 60-70%) is observed in the propeptide domain.

For the purposes of the present disclosure, the term “lysyl oxidase-type enzyme” encompasses all five of the lysine oxidizing enzymes discussed above (LOX, LOXL1, LOXL2, LOXL3 and LOXL4), and also encompasses functional fragments and/or derivatives of LOX, LOXL1, LOXL2, LOXL3 and LOXL4 that substantially retain enzymatic activity; e.g., the ability to catalyze deamination of lysyl residues. Typically, a functional fragment or derivative retains at least 50% of its lysine oxidation activity. In some embodiments, a functional fragment or derivative retains at least 60%, at least 70%, at least 80%, at least 90%, at least 95%, at least 99% or 100% of its lysine oxidation activity.

It is also intended that a functional fragment of a lysyl oxidase-type enzyme can include conservative amino acid substitutions (with respect to the native polypeptide sequence) that do not substantially alter catalytic activity. The term “conservative amino acid substitution” refers to grouping of amino acids on the basis of certain common structures and/or properties. With respect to common structures, amino acids can be grouped into those with non-polar side chains (glycine, alanine, valine, leucine, isoleucine, methionine, proline, phenylalanine and tryptophan), those with uncharged polar side chains (serine, threonine, asparagine, glutamine, tyrosine and cysteine) and those with charged polar side chains (lysine, arginine, aspartic acid, glutamic acid and histidine). A group of amino acids containing aromatic side chains includes phenylalanine, tryptophan and tyrosine. Heterocyclic side chains are present in proline, tryptophan and histidine. Within the group of amino acids containing non-polar side chains, those with short hydrocarbon side chains (glycine, alanine, valine, leucine, isoleucine) can be distinguished from those with longer, non-hydrocarbon side chains (methionine, proline, phenylalanine, tryptophan). Within the group of amino acids with charged polar side chains, the acidic amino acids (aspartic acid, glutamic acid) can be distinguished from those with basic side chains (lysine, arginine and histidine).

A functional method for defining common properties of individual amino acids is to analyze the normalized frequencies of amino acid changes between corresponding proteins of homologous organisms (Schulz, G. E. and R. H. Schirmer, Principles of Protein Structure, Springer-Verlag, 1979). According to such analyses, groups of amino acids can be defined in which amino acids within a group are preferentially substituted for one another in homologous proteins, and therefore have similar impact on overall protein structure (Schulz, G. E. and R. H. Schirmer, Principles of Protein Structure, Springer-Verlag, 1979). According to this type of analysis, the following groups of amino acids that can be conservatively substituted for one another can be identified:

(i) amino acids containing a charged group, consisting of Glu, Asp, Lys, Arg and His,

(ii) amino acids containing a positively-charged group, consisting of Lys, Arg and His,

(iii) amino acids containing a negatively-charged group, consisting of Glu and Asp,

(iv) amino acids containing an aromatic group, consisting of Phe, Tyr and Trp,

(v) amino acids containing a nitrogen ring group, consisting of His and Trp,

(vi) amino acids containing a large aliphatic non-polar group, consisting of Val, Leu and Ile,

(vii) amino acids containing a slightly-polar group, consisting of Met and Cys,

(viii) amino acids containing a small-residue group, consisting of Ser, Thr, Asp, Asn, Gly, Ala, Glu, Gln and Pro,

(ix) amino acids containing an aliphatic group consisting of Val, Leu, Be, Met and Cys, and

(x) amino acids containing a hydroxyl group consisting of Ser and Thr.

Thus, as exemplified above, conservative substitutions of amino acids are known to those of skill in this art and can be made generally without altering the biological activity of the resulting molecule. Those of skill in this art also recognize that, in general, single amino acid substitutions in non-essential regions of a polypeptide do not substantially alter biological activity. See, e.g., Watson, et al., “Molecular Biology of the Gene,” 4th Edition, 1987, The Benjamin/Cummings Pub. Co., Menlo Park, Calif., p. 224.

For additional information regarding lysyl oxidase-type enzymes, see, e.g., Rucker et al. (1998) Am. J. Clin. Nutr. 67:996 S-1002S and Kagan et al. (2003) J. Cell. Biochem 88:660-672. See also co-owned United States patent application publication Nos. 2009/0053224 (Feb. 26, 2009) and 2009/0104201 (Apr. 23, 2009); the disclosures of which are incorporated by reference herein.

Modulators of the Activity of Lysyl Oxidase-Type Enzymes

Modulators of the activity of lysyl oxidase-type enzymes include both activators (agonists) and inhibitors (antagonists), and can be selected by using a variety of screening assays. In one embodiment, modulators can be identified by determining if a test compound binds to a lysyl oxidase-type enzyme; wherein, if binding has occurred, the compound is a candidate modulator. Optionally, additional tests can be carried out on such a candidate modulator. Alternatively, a candidate compound can be contacted with a lysyl oxidase-type enzyme, and a biological activity of the lysyl oxidase-type enzyme assayed; a compound that alters the biological activity of the lysyl oxidase-type enzyme is a modulator of a lysyl oxidase-type enzyme. Generally, a compound that reduces a biological activity of a lysyl oxidase-type enzyme is an inhibitor of the enzyme.

Other methods of identifying modulators of the activity of lysyl oxidase-type enzymes include incubating a candidate compound in a cell culture containing one or more lysyl oxidase-type enzymes and assaying one or more biological activities or characteristics of the cells. Compounds that alter the biological activity or characteristic of the cells in the culture are potential modulators of the activity of a lysyl oxidase-type enzyme. Biological activities that can be assayed include, for example, lysine oxidation, peroxide production, ammonia production, levels of lysyl oxidase-type enzyme, levels of mRNA encoding a lysyl oxidase-type enzyme, and/or one or more functions specific to a lysyl oxidase-type enzyme. In additional embodiments of the aforementioned assay, in the absence of contact with the candidate compound, the one or more biological activities or cell characteristics are correlated with levels or activity of one or more lysyl oxidase-type enzymes. For example, the biological activity can be a cellular function such as migration, chemotaxis, epithelial-to-mesenchymal transition, or mesenchymal-to-epithelial transition, and the change is detected by comparison with one or more control or reference sample(s). For example, negative control samples can include a culture with decreased levels of a lysyl oxidase-type enzyme to which the candidate compound is added; or a culture with the same amount of lysyl oxidase-type enzyme as the test culture, but without addition of candidate compound. In some embodiments, separate cultures containing different levels of a lysyl oxidase-type enzyme are contacted with a candidate compound. If a change in biological activity is observed, and if the change is greater in the culture having higher levels of lysyl oxidase-type enzyme, the compound is identified as a modulator of the activity of a lysyl oxidase-type enzyme. Determination of whether the compound is an activator or an inhibitor of a lysyl oxidase-type enzyme may be apparent from the phenotype induced by the compound, or may require further assay, such as a test of the effect of the compound on the enzymatic activity of one or more lysyl oxidase-type enzymes.

Methods for obtaining lysysl oxidase-type enzymes, either biochemically or recombinantly, as well as methods for cell culture and enzymatic assay to identify modulators of the activity of lysyl oxidase-type enzymes as described above, are known in the art.

The enzymatic activity of a lysyl oxidase-type enzyme can be assayed by a number of different methods. For example, lysyl oxidase enzymatic activity can be assessed by detecting and/or quantitating production of hydrogen peroxide, ammonium ion, and/or aldehyde, by assaying lysine oxidation and/or collagen crosslinking, or by measuring cellular invasive capacity, cell adhesion, cell growth or metastatic growth. See, for example, Trackman et al. (1981) Anal. Biochem. 113:336-342; Kagan et al. (1982) Meth. Enzymol. 82A:637-649; Palamakumbura et al. (2002) Anal. Biochem. 300:245-251; Albini et al. (1987) Cancer Res. 47:3239-3245; Kamath et al. (2001) Cancer Res. 61:5933-5940; U.S. Pat. No. 4,997,854 and U.S. patent application publication No. 2004/0248871.

Test compounds include, but are not limited to, small organic compounds (e.g., organic molecules having a molecular weight between about 50 and about 2,500 Da), nucleic acids or proteins, for example. The compound or plurality of compounds can be chemically synthesized or microbiologically produced and/or comprised in, for example, samples, e.g., cell extracts from, e.g., plants, animals or microorganisms. Furthermore, the compound(s) can be known in the art but hitherto not known to be capable of modulating the activity of a lysyl oxidase-type enzyme. The reaction mixture for assaying for a modulator of a lysyl oxidase-type enzyme can be a cell-free extract or can comprise a cell culture or tissue culture. A plurality of compounds can be, e.g., added to a reaction mixture, added to a culture medium, injected into a cell or administered to a transgenic animal. The cell or tissue employed in the assay can be, for example, a bacterial cell, a fungal cell, an insect cell, a vertebrate cell, a mammalian cell, a primate cell, a human cell or can comprise or be obtained from a non-human transgenic animal.

Several methods are known to the person skilled in the art for producing and screening large libraries to identify compounds having specific affinity for a target, such as a lysyl oxidase-type enzyme. These methods include phage display method in which randomized peptides are displayed from phage and screened by affinity chromatography using an immobilized receptor. See, e.g., WO 91/17271, WO 92/01047, and U.S. Pat. No. 5,223,409. In another approach, combinatorial libraries of polymers immobilized on a solid support (e.g., a “chip”) are synthesized using photolithography. See, e.g., U.S. Pat. No. 5,143,854, WO 90/15070 and WO 92/10092. The immobilized polymers are contacted with a labeled receptor (e.g., a lysyl oxidase-type enzyme) and the support is scanned to determine the location of label, to thereby identify polymers binding to the receptor.

The synthesis and screening of peptide libraries on continuous cellulose membrane supports that can be used for identifying binding ligands of a polypeptide of interest (e.g., a lysyl oxidase-type enzyme) is described, for example, in Kramer (1998) Methods Mol. Biol. 87: 25-39. Ligands identified by such an assay are candidate modulators of the protein of interest, and can be selected for further testing. This method can also be used, for example, for determining the binding sites and the recognition motifs in a protein of interest. See, for example Rudiger (1997) EMBO J. 16:1501-1507 and Weiergraber (1996) FEBS Lett. 379:122-126.

WO 98/25146 describes additional methods for screening libraries of complexes for compounds having a desired property, e.g., the capacity to agonize, bind to, or antagonize a polypeptide or its cellular receptor. The complexes in such libraries comprise a compound under test, a tag recording at least one step in synthesis of the compound, and a tether susceptible to modification by a reporter molecule. Modification of the tether is used to signify that a complex contains a compound having a desired property. The tag can be decoded to reveal at least one step in the synthesis of such a compound. Other methods for identifying compounds which interact with a lysyl oxidase-type enzyme are, for example, in vitro screening with a phage display system, filter binding assays, and “real time” measuring of interaction using, for example, the BIAcore apparatus (Pharmacia).

All these methods can be used in accordance with the present disclosure to identify activators/agonists and inhibitors/antagonists of lysyl oxidase-type enzymes or related polypeptides.

Another approach to the synthesis of modulators of lysyl oxidase-type enzymes is to use mimetic analogs of peptides. Mimetic peptide analogues can be generated by, for example, substituting stereoisomers, i.e. D-amino acids, for naturally-occurring amino acids; see e.g., Tsukida (1997) J. Med. Chem. 40:3534-3541. Furthermore, pro-mimetic components can be incorporated into a peptide to reestablish conformational properties that may be lost upon removal of part of the original polypeptide. See, e.g., Nachman (1995) Regul. Pept. 57:359-370.

Another method for constructing peptide mimetics is to incorporate achiral O-amino acid residues into a peptide, resulting in the substitution of amide bonds by polymethylene units of an aliphatic chain. Banerjee (1996) Biopolymers 39:769-777. Superactive peptidomimetic analogues of small peptide hormones in other systems have been described. Zhang (1996) Biochem. Biophys. Res. Commun. 224:327-331.

Peptide mimetics of a modulator of a lysyl oxidase-type enzyme can also be identified by the synthesis of peptide mimetic combinatorial libraries through successive amide alkylation, followed by testing of the resulting compounds, e.g., for their binding and immunological properties. Methods for the generation and use of peptidomimetic combinatorial libraries have been described. See, for example, Ostresh, (1996) Methods in Enzymology 267:220-234 and Dorner (1996) Bioorg. Med. Chem. 4:709-715. Furthermore, a three-dimensional and/or crystallographic structure of one or more lysyl oxidase-type enzymes can be used for the design of peptide mimetic inhibitors of the activity of one or more lysyl oxidase-type enzymes. Rose (1996) Biochemistry 35:12933-12944; Rutenber (1996) Bioorg. Med. Chem. 4:1545-1558.

The structure-based design and synthesis of low-molecular-weight synthetic molecules that mimic the activity of native biological polypeptides is further described in, e.g., Dowd (1998) Nature Biotechnol. 16:190-195; Kieber-Emmons (1997) Current Opinion Biotechnol. 8:435-441; Moore (1997) Proc. West Pharmacol. Soc. 40:115-119; Mathews (1997) Proc. West Pharmacol. Soc. 40:121-125; and Mukhija (1998) European J. Biochem. 254:433-438.

It is also well known to the person skilled in the art that it is possible to design, synthesize and evaluate mimetics of small organic compounds that, for example, can act as a substrate or ligand of a lysyl oxidase-type enzyme. For example, it has been described that D-glucose mimetics of hapalosin exhibited similar efficiency as hapalosin in antagonizing multidrug resistance assistance-associated protein in cytotoxicity. Dinh (1998) J. Med. Chem. 41:981-987.

The structure of the lysyl oxidase-type enzymes can be investigated to guide the selection of modulators such as, for example, small molecules, peptides, peptide mimetics and antibodies. Structural properties of a lysyl oxidase-type enzyme can help to identify natural or synthetic molecules that bind to, or function as a ligand, substrate, binding partner or the receptor of, the lysyl oxidase-type enzyme. See, e.g., Engleman (1997) J. Clin. Invest. 99:2284-2292. For example, folding simulations and computer redesign of structural motifs of lysyl oxidase-type enzymes can be performed using appropriate computer programs. Olszewski (1996) Proteins 25:286-299; Hoffman (1995) Comput. Appl. Biosci. 11:675-679. Computer modeling of protein folding can be used for the conformational and energetic analysis of detailed peptide and protein structure. Monge (1995) J. Mol. Biol. 247:995-1012; Renouf (1995) Adv. Exp. Med. Biol. 376:37-45. Appropriate programs can be used for the identification of sites, on lysyl oxidase-type enzymes, that interact with ligands and binding partners, using computer assisted searches for complementary peptide sequences. Fassina (1994) Immunomethods 5:114-120. Additional systems for the design of protein and peptides are described, for example in Berry (1994) Biochem. Soc. Trans. 22:1033-1036; Wodak (1987), Ann. N.Y. Acad. Sci. 501:1-13; and Pabo (1986) Biochemistry 25:5987-5991. The results obtained from the above-described structural analyses can be used for, e.g., the preparation of organic molecules, peptides and peptide mimetics that function as modulators of the activity of one or more lysyl oxidase-type enzymes.

An inhibitor of a lysyl oxidase-type enzyme can be a competitive inhibitor, an uncompetitive inhibitor, a mixed inhibitor or a non-competitive inhibitor. Competitive inhibitors often bear a structural similarity to substrate, usually bind to the active site, and are more effective at lower substrate concentrations. The apparent K_{M} is increased in the presence of a competitive inhibitor. Uncompetitive inhibitors generally bind to the enzyme-substrate complex or to a site that becomes available after substrate is bound at the active site and may distort the active site. Both the apparent K_{M} and the Vₘₐₓ are decreased in the presence of an uncompetitive inhibitor, and substrate concentration has little or no effect on inhibition. Mixed inhibitors are capable of binding both to free enzyme and to the enzyme-substrate complex and thus affect both substrate binding and catalytic activity. Non-competitive inhibition is a special case of mixed inhibition in which the inhibitor binds enzyme and enzyme-substrate complex with equal avidity, and inhibition is not affected by substrate concentration. Non-competitive inhibitors generally bind to enzyme at a region outside the active site. For additional details on enzyme inhibition see, for example, Voet et al. (2008) supra. For enzymes such as the lysyl oxidase-type enzymes, whose natural substrates (e.g., collagen, elastin) are normally present in vast excess in vivo (compared to the concentration of any inhibitor that can be achieved in vivo), noncompetitive inhibitors are advantageous, since inhibition is independent of substrate concentration.

Antibodies

In certain embodiments, a modulator of a lysyl oxidase-type enzyme is an antibody. In additional embodiments, an antibody is an inhibitor of the activity of a lysyl oxidase-type enzyme.

As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce clinically significant production of antibodies to itself).

An “antibody fragment” comprises a portion of a full-length antibody, for example, the antigen binding or variable region of a full-length antibody. Examples of antibody fragments include Fab, Fab′, F(ab′)₂, and Fv fragments; diabodies; linear antibodies (Zapata et al. (1995) Protein Eng. 8(10): 1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although generally at a lower affinity than does the entire Fᵥ fragment.

The “F_{ab}” fragment also contains, in addition to heavy and light chain variable regions, the constant domain of the light chain and the first constant domain (CH₁) of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

In some embodiments, an antibody is a humanized antibody or a human antibody. Humanized antibodies include human immununoglobulins (recipient antibody) in which residues from a complementary determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen-binding subsequences of antibodies.

The humanized antibody can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different lysyl oxidase-type enzymes, or to two different epitopes on a single lysyl oxidase-type enzyme.

An antibody as disclosed herein can also be an immunoconjugate. Such immunoconjugates comprise an antibody (e.g., to a lysyl oxidase-type enzyme) conjugated to a second molecule, such as a reporter An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (e.g., to provide a radioconjugate).

An antibody that “specifically binds to” or is “specific for” a particular polypeptide or an epitope on a particular polypeptide is one that binds to that particular polypeptide or epitope without substantially binding to any other polypeptide or polypeptide epitope. In some embodiments, an antibody of the present disclosure specifically binds to its target with a dissociation constant (K_{d}) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; in the form of monoclonal antibody, scFv, Fab, or other form of antibody measured at a temperature of about 4° C., 25° C., 37° C. or 42° C.

In certain embodiments, an antibody of the present disclosure binds to one or more processing sites (e.g., sites of proteolytic cleavage) in a lysyl oxidase-type enzyme, thereby effectively blocking processing of the proenzyme or preproenzyme to the catalytically active enzyme, thereby reducing the activity of the lysyl oxidase-type enzyme.

In certain embodiments, an antibody according to the present disclosure binds to human LOX and/or human LOXL2, with a greater binding affinity, for example, 10 times, at least 100 times, or even at least 1000 times greater, than its binding affinity to other lysyl oxidase-type enzymes, e.g., LOXL1, LOXL3, and LOXL4.

In certain embodiments, an antibody according to the present disclosure is a non-competitive inhibitor of the catalytic activity of a lysyl oxidase-type enzyme. In certain embodiments, an antibody according to the present disclosure binds outside the catalytic domain of a lysyl oxidase-type enzyme. In certain embodiments, an antibody according to the present disclosure binds to the SRCR4 domain of LOXL2. In certain embodiments, an anti-LOXL2 antibody that binds to the SRCR4 domain of LOXL2 and functions as a non-competitive inhibitor is the AB0023 antibody, described herein and in co-owned U.S. Patent Application Publications No. US 2009/0053224 and US 2009/0104201. In certain embodiments, an anti-LOXL2 antibody that binds to the SRCR4 domain of LOXL2 and functions as a non-competitive inhibitor is the AB0024 antibody (a human version of the AB0023 antibody), described herein and in co-owned U.S. Patent Application Publications No. US 2009/0053224 and US 2009/0104201.

Optionally, an antibody according to the present disclosure not only binds to a lysyl oxidase-type enzyme but also reduces or inhibits uptake or internalization of the lysyl oxidase-type enzyme, e.g., via integrin beta 1 or other cellular receptors or proteins. Such an antibody could, for example, bind to extracellular matrix proteins, cellular receptors, and/or integrins.

Exemplary antibodies that recognize lysyl oxidase-type enzymes, and additional disclosure relating to antibodies to lysyl oxidase-type enzymes, is provided in co-owned U.S. Patent Application Publications No. US 2009/0053224 and US 2009/0104201, the disclosures of which are incorporated by reference for the purposes of describing antibodies to lysyl oxidase-type enzymes, their manufacture, and their use.

Polynucleotides for Modulating Expression of Lysyl Oxidase-Type Enzymes

Antisense

Modulation (e.g., inhibition) of a lysyl oxidase-type enzyme can be effected by down-regulating expression of the lysyl oxidase enzyme at either the transcriptional or translational level. One such method of modulation involves the use of antisense oligo- or polynucleotides capable of sequence-specific binding with a mRNA transcript encoding a lysyl oxidase-type enzyme.

Binding of an antisense oligonucleotide (or antisense oligonucleotide analogue) to a target mRNA molecule can lead to the enzymatic cleavage of the hybrid by intracellular RNase H. In certain cases, formation of an antisense RNA-mRNA hybrid can interfere with correct splicing. In both cases, the number of intact, functional target mRNAs, suitable for translation, is reduced or eliminated. In other cases, binding of an antisense oligonucleotide or oligonucleotide analogue to a target mRNA can prevent (e.g., by steric hindrance) ribosome binding, thereby preventing translation of the mRNA.

Antisense oligonucleotides can comprise any type of nucleotide subunit, e.g., they can be DNA, RNA, analogues such as peptide nucleic acids (PNA), or mixtures of the preceding. RNA oligonucleotides form a more stable duplex with a target mRNA molecule, but the unhybridized oligonucleotides are less stable intracellularly than other types of oligonucleotides and oligonucleotide analogues. This can be counteracted by expressing RNA oligonucleotides inside a cell using vectors designed for this purpose. This approach may be used, for example, when attempting to target a mRNA that encodes an abundant and long-lived protein.

Additional considerations can be taken into account when designing antisense oligonucleotides, including: (i) sufficient specificity in binding to the target sequence; (ii) solubility; (iii) stability against intra- and extracellular nucleases; (iv) ability to penetrate the cell membrane; and (v) when used to treat an organism, low toxicity.

Algorithms for identifying oligonucleotide sequences with the highest predicted binding affinity for their target mRNA, based on a thermodynamic cycle that accounts for the energy of structural alterations in both the target mRNA and the oligonucleotide, are available. For example, Walton et al. (1999) Biotechnol. Bioeng. 65:1-9 used such a method to design antisense oligonucleotides directed to rabbit β-globin (RBG) and mouse tumor necrosis factor-α (TNF α) transcripts. The same research group has also reported that the antisense activity of rationally selected oligonucleotides against three model target mRNAs (human lactate dehydrogenase A and B and rat gp130) in cell culture proved effective in almost all cases. This included tests against three different targets in two cell types using oligonucleotides made by both phosphodiester and phosphorothioate chemistries.

In addition, several approaches for designing and predicting efficiency of specific oligonucleotides using an in vitro system are available. See, e.g., Matveeva et al. (1998) Nature Biotechnology 16:1374-1375.

An antisense oligonucleotide according to the present disclosure includes a polynucleotide or a polynucleotide analogue of at least 10 nucleotides, for example, between 10 and 15, between 15 and 20, at least 17, at least 18, at least 19, at least 20, at least 22, at least 25, at least 30, or even at least 40 nucleotides. Such a polynucleotide or polynucleotide analogue is able to anneal or hybridize (i.e., form a double-stranded structure on the basis of base complementarity) in vivo, under physiological conditions, with a mRNA encoding a lysyl oxidase-type enzyme, e.g., LOX or LOXL2.

Antisense oligonucleotides according to the present disclosure can be expressed from a nucleic acid construct administered to a cell or tissue. Optionally, expression of the antisense sequences is controlled by an inducible promoter, such that expression of antisense sequences can be switched on and off in a cell or tissue. Alternatively antisense oligonucleotides can be chemically synthesized and administered directly to a cell or tissue, as part of, for example, a pharmaceutical composition.

Antisense technology has led to the generation of highly accurate antisense design algorithms and a wide variety of oligonucleotide delivery systems, thereby enabling those of ordinary skill in the art to design and implement antisense approaches suitable for downregulating expression of known sequences. For additional information relating to antisense technology, see, for example, Lichtenstein et al., “Antisense Technology: A Practical Approach,” Oxford University Press, 1998.

Small RNA and RNAi

Another method for inhibition of the activity of a lysyl oxidase-type enzyme is RNA interference (RNAi), an approach which utilizes double-stranded small interfering RNA (siRNA) molecules that are homologous to a target mRNA and lead to its degradation. Carthew (2001) Curr. Opin. Cell. Biol. 13:244-248.

RNA interference is typically a two-step process. In the first step, which is termed as the initiation step, input dsRNA is digested into 21-23 nucleotide (nt) small interfering RNAs (siRNAs), probably by the action of Dicer, a member of the RNase III family of double-strand-specific ribonucleases, which cleaves double-stranded RNA in an ATP-dependent manner. Input RNA can be delivered, e.g., directly or via a transgene or a virus. Successive cleavage events degrade the RNA to 19-21 bp duplexes (siRNA), each with 2-nucleotide 3′ overhangs. Hutvagner et al. (2002) Curr. Opin. Genet. Dev. 12:225-232; Bernstein (2001) Nature 409:363-366.

In the second, effector step, siRNA duplexes bind to a nuclease complex to form the RNA-induced silencing complex (RISC). An ATP-dependent unwinding of the siRNA duplex is required for activation of the RISC. The active RISC (containing a single siRNA and an RNase) then targets the homologous transcript by base pairing interactions and typically cleaves the mRNA into fragments of approximately 12 nucleotides, starting from the 3′ terminus of the siRNA. Hutvagner et al., supra; Hammond et al. (2001) Nat. Rev. Gen. 2:110-119; Sharp (2001) Genes. Dev. 15:485-490.

RNAi and associated methods are also described in Tuschl (2001) Chem. Biochem. 2:239-245; Cullen (2002) Nat. Immunol. 3:597-599; and Brantl (2002) Biochem. Biophys. Acta. 1575:15-25.

An exemplary strategy for synthesis of RNAi molecules suitable for use with the present disclosure, as inhibitors of the activity of a lysyl oxidase-type enzyme, is to scan the appropriate mRNA sequence downstream of the start codon for AA dinucleotide sequences. Each AA, plus the downstream (i.e., 3′ adjacent) 19 nucleotides, is recorded as a potential siRNA target site. Target sites in coding regions are preferred, since proteins that bind in untranslated regions (UTRs) of a mRNA, and/or translation initiation complexes, may interfere with binding of the siRNA endonuclease complex. Tuschl (2001) supra. It will be appreciated though, that siRNAs directed at untranslated regions can also be effective, as has been demonstrated in the case wherein siRNA directed at the 5′ UTR of the GAPDH gene mediated about 90% decrease in cellular GAPDH mRNA and completely abolished protein level (www.ambion.com/techlib/tn/91/912.html). Once a set of potential target sites is obtained, as described above, the sequences of the potential targets are compared to an appropriate genomic database (e.g., human, mouse, rat etc.) using a sequence alignment software, (such as the BLAST software available from NCBI at www.ncbi.nlm.nih.gov/BLAST/). Potential target sites that exhibit significant homology to other coding sequences are rejected.

Qualifying target sequences are selected as templates for siRNA synthesis. Selected sequences can include those with low G/C content as these have been shown to be more effective in mediating gene silencing, compared to those with G/C content higher than 55%. Several target sites can be selected along the length of the target gene for evaluation. For better evaluation of the selected siRNAs, a negative control is used in conjunction. Negative control siRNA can include a sequence with the same nucleotide composition as a test siRNA, but lacking significant homology to the genome. Thus, for example, a scrambled nucleotide sequence of the siRNA may be used, provided it does not display any significant homology to any other gene.

The siRNA molecules of the present disclosure can be transcribed from expression vectors which can facilitate stable expression of the siRNA transcripts once introduced into a host cell. These vectors are engineered to express small hairpin RNAs (shRNAs), which are processed in vivo into siRNA molecules capable of carrying out gene-specific silencing. See, for example, Brummelkamp et al. (2002) Science 296:550-553; Paddison et al (2002) Genes Dev. 16:948-958; Paul et al. (2002) Nature Biotech. 20:505-508; Yu et al. (2002) Proc. Natl. Acad. Sci. USA 99:6047-6052.

Small hairpin RNAs (shRNAs) are single-stranded polynucleotides that form a double-stranded, hairpin loop structure. The double-stranded region is formed from a first sequence that is hybridizable to a target sequence, such as a polynucleotide encoding a lysyl oxidase-type enzyme (e.g., a LOX or LOXL2 mRNA) and a second sequence that is complementary to the first sequence. The first and second sequences form a double stranded region; while the un-base-paired linker nucleotides that lie between the first and second sequences form a hairpin loop structure. The double-stranded region (stem) of the shRNA can comprise a restriction endonuclease recognition site.

A shRNA molecule can have optional nucleotide overhangs, such as 2-bp overhangs, for example, 3′ UU-overhangs. While there may be variation, stem length typically ranges from approximately 15 to 49, approximately 15 to 35, approximately 19 to 35, approximately 21 to 31 bp, or approximately 21 to 29 bp, and the size of the loop can range from approximately 4 to 30 bp, for example, about 4 to 23 bp.

For expression of shRNAs within cells, plasmid vectors can be employed that contain a promoter (e.g., the RNA Polymerase III H1-RNA promoter or the U6 RNA promoter), a cloning site for insertion of sequences encoding the shRNA, and a transcription termination signal (e.g., a stretch of 4-5 adenine-thymidine base pairs). Polymerase III promoters generally have well-defined transcriptional initiation and termination sites, and their transcripts lack poly(A) tails. The termination signal for these promoters is defined by the polythymidine tract, and the transcript is typically cleaved after the second encoded uridine. Cleavage at this position generates a 3′ UU overhang in the expressed shRNA, which is similar to the 3′ overhangs of synthetic siRNAs. Additional methods for expressing shRNA in mammalian cells are described in the references cited above.

An example of a suitable shRNA expression vector is pSUPER™ (Oligoengine, Inc., Seattle, Wash.), which includes the polymerase-III H1-RNA gene promoter with a well defined transcriptional startsite and a termination signal consisting of five consecutive adenine-thymidine pairs. Brummelkamp et al., supra. The transcription product is cleaved at a site following the second uridine (of the five encoded by the termination sequence), yielding a transcript which resembles the ends of synthetic siRNAs, which also contain nucleotide overhangs. Sequences to be transcribed into shRNA are cloned into such a vector such that they will generate a transcript comprising a first sequence complementary to a portion of a mRNA target (e.g., a mRNA encoding a lysyl oxidase-type enzyme), separated by a short spacer from a second sequence comprising the reverse complement of the first sequence. The resulting transcript folds back on itself to form a stem-loop structure, which mediates RNA interference (RNAi).

Another suitable siRNA expression vector encodes sense and antisense siRNA under the regulation of separate pol III promoters. Miyagishi et al. (2002) Nature Biotech. 20:497-500. The siRNA generated by this vector also includes a five thymidine (T5) termination signal.

siRNAs, shRNAs and/or vectors encoding them can be introduced into cells by a variety of methods, e.g., lipofection. Vector-mediated methods have also been developed. For example, siRNA molecules can be delivered into cells using retroviruses. Delivery of siRNA using retroviruses can provide advantages in certain situations, since retroviral delivery can be efficient, uniform and immediately selects for stable “knock-down” cells. Devroe et al. (2002) BMC Biotechnol. 2:15.

Recent scientific publications have validated the efficacy of such short double stranded RNA molecules in inhibiting target mRNA expression and thus have clearly demonstrated the therapeutic potential of such molecules. For example, RNAi has been utilized for inhibition in cells infected with hepatitis C virus (McCaffrey et al. (2002) Nature 418:38-39), HIV-1 infected cells (Jacque et al. (2002) Nature 418:435-438), cervical cancer cells (Jiang et al. (2002) Oncogene 21:6041-6048) and leukemic cells (Wilda et al. (2002) Oncogene 21:5716-5724).

Methods for Modulating Expression of Lysyl Oxidase-Type Enzymes

Another method for modulating the activity of a lysyl oxidase-type enzyme is to modulate the expression of its encoding gene, leading to lower levels of activity if gene expression is repressed, and higher levels if gene expression is activated. Modulation of gene expression in a cell can be achieved by a number of methods.

For example, oligonucleotides that bind genomic DNA (e.g., regulatory regions of a lysyl oxidase-type gene) by strand displacement or by triple-helix formation can block transcription, thereby preventing expression of a lysyl oxidase-type enzyme. In this regard, the use of so-called “switch back” chemical linking, in which an oligonucleotide recognizes a polypurine stretch on one strand on one strand of its target and a homopurine sequence on the other strand, has been described. Triple-helix formation can also be obtained using oligonucleotides containing artificial bases, thereby extending binding conditions with regard to ionic strength and pH.

Modulation of transcription of a gene encoding a lysyl oxidase-type enzyme can also be achieved, for example, by introducing into cell a fusion protein comprising a functional domain and a DNA-binding domain, or a nucleic acid encoding such a fusion protein. A functional domain can be, for example, a transcriptional activation domain or a transcriptional repression domain. Exemplary transcriptional activation domains include VP16, VP64 and the p65 subunit of NF-κB; exemplary transcriptional repression domains include KRAB, KOX and v-erbA.

In certain embodiments, the DNA-binding domain portion of such a fusion protein is a sequence-specific DNA-binding domain that binds in or near a gene encoding a lysyl oxidase-type enzyme, or in a regulatory region of such a gene. The DNA-binding domain can either naturally bind to a sequence at or near the gene or regulatory region, or can be engineered to so bind. For example, the DNA-binding domain can be obtained from a naturally-occurring protein that regulates expression of a gene encoding a lysyl oxidase-type enzyme. Alternatively, the DNA-binding domain can be engineered to bind to a sequence of choice in or near a gene encoding a lysyl oxidase-type enzyme or in a regulatory region of such a gene.

In this regard, the zinc finger DNA-binding domain is useful, inasmuch as it is possible to engineer zinc finger proteins to bind to any DNA sequence of choice. A zinc finger binding domain comprises one or more zinc finger structures. Miller et al. (1985) EMBO J. 4:1609-1614; Rhodes (1993) Scientific American, February: 56-65; U.S. Pat. No. 6,453,242. Typically, a single zinc finger is about 30 amino acids in length and contains four zinc-coordinating amino acid residues. Structural studies have demonstrated that the canonical (C₂H₂) zinc finger motif contains two beta sheets (held in a beta turn which generally contains two zinc-coordinating cysteine residues) packed against an alpha helix (generally containing two zinc coordinating histidine residues).

Zinc fingers include both canonical C₂H₂ zinc fingers (i.e., those in which the zinc ion is coordinated by two cysteine and two histidine residues) and non-canonical zinc fingers such as, for example, C₃H zinc fingers (those in which the zinc ion is coordinated by three cysteine residues and one histidine residue) and C₄ zinc fingers (those in which the zinc ion is coordinated by four cysteine residues). Non-canonical zinc fingers can also include those in which an amino acid other than cysteine or histidine is substituted for one of these zinc-coordinating residues. See e.g., WO 02/057293 (Jul. 25, 2002) and US 2003/0108880 (Jun. 12, 2003).

Zinc finger binding domains can be engineered to have a novel binding specificity, compared to a naturally-occurring zinc finger protein; thereby allowing the construction of zinc finger binding domains engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. Engineering methods include, but are not limited to, rational design and various types of empirical selection methods.

Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,030,215; 7,067,617; U.S. Patent Application Publication Nos. 2002/0165356; 2004/0197892; 2007/0154989; 2007/0213269; and International Patent Application Publication Nos. WO 98/53059 and WO 2003/016496.

Exemplary selection methods, including phage display, interaction trap, hybrid selection and two-hybrid systems, are disclosed in U.S. Pat. Nos. 5,789,538; 5,925,523; 6,007,988; 6,013,453; 6,140,466; 6,200,759; 6,242,568; 6,410,248; 6,733,970; 6,790,941; 7,029,847 and 7,297,491; as well as U.S. Patent Application Publication Nos. 2007/0009948 and 2007/0009962; WO 98/37186; WO 01/60970 and GB 2,338,237.

Enhancement of binding specificity for zinc finger binding domains has been described, for example, in U.S. Pat. No. 6,794,136 (Sep. 21, 2004). Additional aspects of zinc finger engineering, with respect to inter-finger linker sequences, are disclosed in U.S. Pat. No. 6,479,626 and U.S. Patent Application Publication No. 2003/0119023. See also Moore et al. (2001a) Proc. Natl. Acad. Sci. USA 98:1432-1436; Moore et al. (2001b) Proc. Natl. Acad. Sci. USA 98:1437-1441 and WO 01/53480.

Further details on the use of fusion proteins comprising engineered zinc finger DNA-binding domains are found, for example, in U.S. Pat. Nos. 6,534,261; 6,607,882; 6,824,978; 6,933,113; 6,979,539; 7,013,219; 7,070,934; 7,163,824 and 7,220,719.

Additional methods for modulating the expression of a lysyl oxidase-type enzyme include targeted mutagenesis, either of the gene or of a regulatory region that controls expression of the gene. Exemplary methods for targeted mutagenesis using fusion proteins comprising a nuclease domain and an engineered DNA-binding domain are provided, for example, in U.S. patent application publications 2005/0064474; 2007/0134796; and 2007/0218528.

Formulations, Kits and Routes of Administration

Therapeutic compositions comprising compounds identified as modulators of the activity of a lysyl oxidase-type enzyme (e.g., inhibitors or activators of a lysyl oxidase-type enzyme) are also provided. Such compositions typically comprise the modulator and a pharmaceutically acceptable carrier. Supplementary active compounds can also be incorporated into the compositions. Modulators, particularly inhibitors, of the activity of a lysyl oxidase-type enzyme can be used, for example, in combination with a chemotherapeutic or anti-neoplastic agent to reduce or eliminate desmoplasia and/or fibroblast activation, for example. Accordingly, therapeutic compositions as disclosed herein can contain both a modulator of the activity of a lysyl oxidase-type enzyme and one or more chemotherapeutic or anti-neoplastic agents. In additional embodiments, therapeutic compositions comprise a therapeutically effective amount of a modulator of the activity of a lysyl oxidase-type enzyme, but do not contain a chemotherapeutic or anti-neoplastic agent, and the compositions are administered separately from the chemotherapeutic or anti-neoplastic agent.

As used herein, the term “therapeutically effective amount” or “effective amount” refers to an amount of a therapeutic agent that when administered alone or in combination with another therapeutic agent to a cell, tissue, or subject (e.g., a mammal such as a human or a non-human animal such as a primate, rodent, cow, horse, pig, sheep, etc.) is effective to prevent or ameliorate the disease condition or the progression of the disease. A therapeutically effective dose further refers to that amount of the compound sufficient to result in full or partial amelioration of symptoms, e.g., treatment, healing, prevention or amelioration of the relevant medical condition, or an increase in rate of treatment, healing, prevention or amelioration of such conditions. A therapeutically effective amount of, for example, an inhibitor of the activity of a lysyl oxidase-type enzyme varies with the type of disease or disorder, extensiveness of the disease or disorder, and size of the organism suffering from the disease or disorder.

The therapeutic compositions disclosed herein are useful for, inter alia, reducing desmoplasia resulting from tumor growth and/or fibrosis. Accordingly, a “therapeutically effective amount” of a modulator (e.g., inhibitor) of the activity of a lysyl oxidase-type enzyme (e.g., LOXL2) is an amount that results in reduction of desmoplasia and/or symptoms associated with desmoplasia. For example, when the inhibitor of a lysyl oxidase enzyme is an antibody and the antibody is administered in vivo, normal dosage amounts may vary from about 10 ng/kg to up to 100 mg/kg of mammal body weight or more per day, for example, about 1 μg/kg/day to 50 mg/kg/day, e.g., about 30 mg/kg/day, optionally about 100 μg/kg/day to 20 mg/kg/day, 500 μg/kg/day to 10 mg/kg/day, or 1 mg/kg/day to 10 mg/kg/day, depending upon, e.g., body weight, route of administration, severity of disease, etc. Dosage amounts can also be administered rather than daily on a schedule of, for example, once a week, twice per week, three times per week, once every 10 days, once every two weeks, or once a month. Dosages can be in an amount of, for example, from about 10 ng/kg to up to 100 mg/kg of mammal body weight or more per dose, for example, about 1 μg/kg/dose to 50 mg/kg/dose, e.g., a bout 30 mg/kg/dose, optionally about 100 μg/kg/dose to 20 mg/kg/dose, 500 μg/kg/dose to 10 mg/kg/dose, or 1 mg/kg/dose to 10 mg/kg/dose, or about 15 mg/kg/dose. In one example, the dose is about 15/mg/kg administered twice weekly. The periods of treatment can range from, for example, 2 weeks, 3 weeks, 4 weeks, 5 weeks, 6 weeks, 7 weeks, 8 weeks, 9 weeks, 10 weeks, 11 weeks, 12 weeks, 13 weeks or 14 weeks, or more. Dosage regimen can include administration of a dose (e.g., from about 10 ng/kg to up to 100 mg/kg of mammal body weight or more per dose, for example, about 1 μg/kg/dose to 50 mg/kg/dose, e.g., a bout 30 mg/kg/dose, optionally about 100 μg/kg/dose to 20 mg/kg/dose, 500 μg/kg/dose to 10 mg/kg/dose, or 1 mg/kg/dose to 10 mg/kg/dose, or about 15 mg/kg/dose) every two weeks.

When a modulator of the activity of a lysyl oxidase-type enzyme is used in combination with a chemotherapeutic or anti-neoplastic agent, one can also refer to the therapeutically effective dose of the combination, which is the combined amounts of the modulator and the chemotherapeutic or anti-neoplastic agent that result in reduction of desmoplasia, whether administered in combination, serially or simultaneously. More than one combination of concentrations can be therapeutically effective.

Various pharmaceutical compositions and techniques for their preparation and use are known to those of skill in the art in light of the present disclosure. For a detailed listing of suitable pharmacological compositions and techniques for their administration one may refer to the detailed teachings herein, which may be further supplemented by texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The disclosed therapeutic compositions further include pharmaceutically acceptable materials, compositions or vehicle, such as a liquid or solid filler, diluent, excipient, solvent or encapsulating material, i.e., carriers. These carriers are involved in transporting the subject modulator from one organ, or region of the body, to another organ, or region of the body. Each carrier should be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Another aspect of the present disclosure relates to kits for carrying out the administration of a modulator of the activity of a lysyl oxidase-type enzyme. Another aspect of the present disclosure relates to kits for carrying out the combined administration of a modulator of the activity of a lysyl oxidase-type enzyme and a chemotherapeutic or anti-neoplastic agent. In one embodiment, a kit comprises an inhibitor of the activity of a lysyl oxidase-type enzyme (e.g. an inhibitor of LOXL2, e.g., an anti-LOXL2 antibody) formulated in a pharmaceutical carrier, optionally containing at least one chemotherapeutic or anti-neoplastic agent, formulated as appropriate, in one or more separate pharmaceutical preparations.

The formulation and delivery methods can be adapted according to the site(s) and degree of desmoplasia. Exemplary formulations include, but are not limited to, those suitable for parenteral administration, e.g., intravenous, intra-arterial, intra-ocular, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments and gels; and other formulations such as inhalants, aerosols and sprays. The dosage of the compounds of the disclosure will vary according to the extent and severity of the need for treatment, the activity of the administered composition, the general health of the subject, and other considerations well known to the skilled artisan.

Therapeutic compositions can be administered to reduce desmoplasia resulting from tumor growth by any suitable route that provides for delivery of the composition to the tumor-stroma interface (i.e., the periphery of the tumor (e.g., the tumor capsule)) along with the adjacent stromal tissue and/or to stromal tissue outside of a tumor.

In additional embodiments, the compositions described herein are delivered locally. Localized delivery allows for the delivery of the composition non-systemically, for example, to a wound or fibrotic area, reducing the body burden of the composition as compared to systemic delivery. Such local delivery can be achieved, for example, through the use of various medically implanted devices including, but not limited to, stents and catheters, or can be achieved by injection or surgery. Methods for coating, implanting, embedding, and otherwise attaching desired agents to medical devices such as stents and catheters are established in the art and contemplated herein.

Anti-LOXL2 Antibodies

A monoclonal antibody directed against LOXL2 has been described in co-owned United States Patent Application Publication No. US 2009/0053224 (Feb. 26, 2009). This antibody is designated AB0023. Antibodies having a heavy chain having the CDRs (CDR1, CDR2, and CDR3) of AB0023 and having a light chain having the CDRs (CDR1, CDR2, and CDR3) of AB0023 are of interest. The sequence of the CDRs and intervening framework regions of the variable region of its heavy chain is as follows (the sequences of CDR1, CDR2, and CDR3 are underlined):

(SEQ ID NO: 1)
MEWSRVFIFLLSVTAGVHSQVQLQQSGAELVRPGTSVKVSCKASGYAF
TYYLIEWVKQRPGQGLEWIGVINPGSGGTNYNEKFKGKATLTADKSSS
TAYMQLSSLTSDDSAVYFCARNWMNFDYWGQGTTLTVSS

Additional heavy chain variable region amino acid sequences having 75% or more, 80% or more, 90% or more, 95% or more, or 99% or more homology to SEQ ID NO:1 are also provided.

The sequence of the CDRs and intervening framework regions of the variable region of the light chain of the AB0023 antibody is (the sequences of CDR1, CDR2, and CDR3 are underlined):

(SEQ ID NO: 2)
MRCLAEFLGLLVLWIPGAIGDIVMTQAAPSVSVTPGESVSISCRSSKS
LLHSNGNTYLYWFLQRPGQSPQFLIYRMSNLASGVPDRFSGSGSGTAF
TLRISRVEAEDVGVYYCMQHLEYPYTFGGGIKLEIK

Additional light chain variable region amino acid sequences having 75% or more, 80% or more, 90% or more, 95% or more, or 99% or more homology to SEQ ID NO:2 are also provided.

Humanized versions of the above-mentioned anti-LOXL2 monoclonal antibody have been described in co-owned United States Patent Application Publication No. US 2009/0053224 (Feb. 26, 2009). An exemplary humanized antibody is designated AB0024. Humanized antibodies having a heavy chain having the CDRs (CDR1, CDR2, and CDR3) of AB0024 and having a light chain having the CDRs (CDR1, CDR2, and CDR3) of AB0024 are of interest. The sequence of the CDRs and intervening framework regions of the variable region of its heavy chain is as follows (the sequences of CDR1, CDR2, and CDR3 are underlined):

(SEQ ID NO: 3)
QVQLVQSGAEVKKPGASVKVSCKASGYAFTYYLIEWVRQAPGQGLEWI
GVINPGSGGTNYNEKFKGRATITADKSTSTAYMELSSLRSEDTAVYFC
ARNWMNFDYWGQGTTVTVSS

Additional heavy chain variable region amino acid sequences having 75% or more, 80% or more, 90% or more, 95% or more, or 99% or more homology to SEQ ID NO:3 are also provided.

The sequence of the CDRs and intervening framework regions of the variable region of the light chain of the AB0024 antibody is (the sequenced of CDR1, CDR2, and CDR3 are underlined):

(SEQ ID NO: 4)
DIVMTQTPLSLSVTPGQPASISCRSSKSLLHSNGNTYLYWFLQKPGQS
PQFLIYRMSNLASGVPDRFSGSGSGTDFTLKISRVEAEDVGVYYCMQH
LEYPYTFGGGTKVEIK

Additional light chain variable region amino acid sequences having 75% or more, 80% or more, 90% or more, 95% or more, or 99% or more homology to SEQ ID NO:4 are also provided.

Additional anti-LOXL2 antibody sequences, including additional humanized variants of the variable regions, framework region amino acid sequences and the amino acid sequences of the complementarity-determining regions, are disclosed in co-owned United States Patent Application Publication No. US 2009/0053224 (Feb. 26, 2009), the disclosure of which is incorporated by reference in its entirety herein for the purpose of providing the amino acid sequences of various anti-LOXL2 antibodies.

### EXAMPLES

### Example 1

### Tissues and Cell Lines

Cell lines were obtained from ATCC (Manassas, Va.) and were maintained in DMEM+10% FBS or serum-free DMEM, depending on the experiment.

### Example 2

### Constructs and Expression Vectors

Generation of Human, Rat, and Cynomolgus Monkey LOXL2 Expression Vectors

Rat LOXL2 was cloned from normal rat cDNA (a mixture of heart, kidney, skeletal muscle and colon cDNA, Biochain Institute) by PCR using Platinum Pfx DNA polymerase (Invitrogen) and primers 5′ atggagatcccttttggctc 3′ (SEQ ID NO:5) and 5′ ttactgcacagagagctgatta3′ (SEQ ID NO:6). 30 PCR cycles were run at 94° C. for 15 seconds, 55° C. for 30 seconds, and 68° C. for 2.5 minutes after an initial incubation at 94° C. for 4 minutes. PCR fragments were gel purified (Gel Extraction Kit, Qiagen) and sequence verified (MCLab, South San Francisco, Calif.). A correct clone was amplified by PCR using primers 5′ atagctagcgccaccatggagatcccttttggctc 3′ (SEQ ID NO:7) and 5′ tatactcgagtctgcacagagagctgattatttag3′ (SEQ ID NO:8) (as previously described, but for 18 cycles), cloned into pSecTag2hygro-B (Invitrogen) at the NheI and XhoI sites, and sequence verified. Cynomolgus monkey LOXL2 was cloned by PCR (as described previously) from normal cDNA (a mixture of stomach, kidney, colon, penis and skeletal muscle cDNA, Biochain Institute) using primers 5′ cctgtcccccctgagcctggcacag3′ (SEQ ID NO:9) and 5′ ttactgcggggagagctggttgttcaagag3′ (SEQ ID NO:10) (generates a fragment with an incomplete signal peptide) and the correct ORF sequence determined by comparison of multiple PCR reactions. This was cloned into the pSecTag2hygro vector using primers 5′ tataggcccagccggcccagtatgacagctggccc3′ (SEQ ID NO:11) and 5′ tatagcggccgcctgcggggagagctggttg3′ (SEQ ID NO:12) at the SfiI and NotI sites (excises endogenous signal peptide), and sequence verified (MCLab). Human LOXL2 was assembled by Genecopoeia (Germantown, Md.) into the pReceiverM08 vector and contains hemagglutinin (HA) and his₆ tags. The catalytically inactive LOXL2, with a Y689F mutation in the lysine tyrosylquinone (LTQ) region was a gift from the Gera Neufeld lab (Technion, the Israel Institute of Technology).

### Example 3

### Antibody Production and Purification

Hybridoma cells were cultured in low IgG DMEM, 10% fetal bovine serum, containing penicillin/streptomycin, 5% hybridoma cloning factor, and HT media supplement. Ascites fluid was produced in BALB/c mice and antibody was purified by packed bed chromatography with MabSelect resin (GE Health). After batch binding, flow-through was collected and the resin was washed with 10 column volumes of PBS, pH 7.4. The antibody was eluted with 0.1M citric acid pH 3. The eluate was neutralized with 1:10 volume 0.1M Tris pH 8.0 and dialyzed overnight at 4° C. in 0.01% Tween 20/PBS.

A mouse anti-human LOXL2 antibody (AB0023) was obtained by immunization with full-length LOXL2 protein and purified by SEC-HPLC (Tosoh TSKGEL G3000SWXL 7.8×300). Analytical size exclusion chromatography was used to assess antibody stability and purity. Purified AB0023 was run on SDS-PAGE Coomassie (Invitrogen BT 4-12% Gels) reducing and non-reducing gels to assess purity. Potency was examined by ELISA to determine K_{d}, and effect on LOXL2 enzymatic activity was determined using an Amplex Red Assay (Molecular Probes/Invitrogen, Carlsbad, Calif.). Antibody concentration was measured by absorbance at 280 nm using an Extinction Coefficient Abs 0.1% of 1.4. Identity was assayed by isoelectric focusing (Invitrogen pH 3-10 IEF Gels). For safety analysis, endotoxin levels were measured at a sensitivity range of 0.01-1 EU/ml (Charles River EndoSafe PTS).

An anti-LOX antibody was obtained by immunization with a peptide having the amino acid sequence DTYERPRPGGRYRPGC (SEQ ID NO:13).

### Example 4

### Purification of LOXL2 and LOXL2 Fragments

Ni-Sepharose (GE Healthcare) resin was equilibrated with 0.1M Tris-HCL pH 8.0. Conditioned medium was loaded onto equilibrated resin. After loading, the nickel affinity column was washed with 0.1M Tris-HCL pH 8.0, 0.25M NaCl, 0.02M Imidazole. Elution was carried out with 0.1M Tris pH 8.0, 0.150M NaCl, 0.3M Imidazole. SDS-PAGE was performed with 4-12% BisTris (Invitrogen) gels on reduced samples to determine purity. Purified protein was then dialyzed overnight at 4° C. in 0.05M Borate pH 8.0.

### Example 5

### Immunofluoresence Assays

Rhodamine Phalloidin Staining

Cells were seeded at 80% confluency, in an 8-chambered slide, 24 hours prior to the day of staining. After 24 hours, media was aspirated and the chambers were washed with PBS. Cells were then fixed with 4% Parafomaldehyde (PFA) for 20 minutes at room temperature and then permeabilized with 0.5% Saponin (JT Baker, Phillipsburg, N.J.) for 5 minutes at room temperature. Cells were then stained for 15 minutes at room temperature with a 1:100 dilution of rhodamine phalloidin (Invitrogen, Carlsbad, Calif.). Slides were mounted with Vectashield (Vector Laboratories, Burlingame, Calif.).

Co-Localization of LOX, LOXL2 and Collagen Type 1: Immunofluorescence

Hs578t cells were seeded in an 8 chamber glass slide (BD Falcon, Franklin Lakes, N.J.) and incubated overnight. For low confluency, cells were seeded at 30-40,000 cells per slide. Low confluency conditions were used for detection of LOX in the cytosol 24 hours after seeding. For high confluency, cells were seeded at 100,000 cells per slide. High confluency conditions were used for detection of matrix-associated LOX, and collagen, approximately 48-72 hours after seeding.

After the cells were incubated for 24 hours, anti-LOX or anti-LOXL2 mAbs were added to the slides, in regular growth medium, to a final concentration of 1 ug/ml, and the slides were incubated for approximately 24 hours. After 24 hours, medium was removed and the cells were rinsed with 1×PBS. The cells were then fixed in 4% paraformaldehyde (PFA) at room temperature for 20 minutes. For collagen detection, anti-collagen antibody (1:50 anti-collagen type I rabbit polyclonal, Calbiochem. Gibbstown, N.J.) was added one hour prior to fixing the cells with 4% PFA and was detected using anti-rabbit Cy3 (ImmunoJackson Labs, West Grove, Pa.) as the secondary Ab.

Cells were permeabilized by addition of saponin buffer (0.5% Saponin/1% BSA in PBS) to the cells at room temperature and incubation for 20 minutes. The secondary antibody (Alexa Fluor 488 donkey anti-mouse IgG, Invitrogen, Carlsbad, Calif.) was added in saponin buffer at room temperature and incubated for 30-45 minutes. The cells were washed three times in saponin buffer and then mounted with Vectashield (Vector Laboratories, Burlingame, Calif.).

### Example 6

### Cell-Based Assays

Preparation of Cell Lysate and Conditioned Medium Samples for Protein Blotting Analysis

Hs578T, MDA-MB-231, MCF7, A549, and HFF cell lines were grown in Dulbecco's modified Eagle's medium (DMEM, Mediatech, Manasas, Va.), supplemented with 10% FBS (PAA, Etobicoke, Ontario, Canada) and L-glutamine (Mediatech, Manasas, Va.). Cells were cultured under normoxic (95% air, 5% CO₂) or hypoxic (2% O₂, 5% CO₂, balanced with N₂) conditions at 37° C. Conditioned medium (DMEM without FBS) was collected and concentrated using an Amicon Ultra-4 (Millipore, Billerica, Mass.). Cells were scraped, vortexed and sonicated in 8 M urea (in 16 mM Na₂HPO₄). Then the cell lysate was concentrated using an Amicon Ultra-15 (Millipore, Billerica, Mass.). Concentrated conditioned media and cell lysates were mixed with SDS sample buffer (Boston Bioproducts, Worcester, Mass.) and boiled at 95° C. for 5 min.

Induction of EMT-like Phenotype by LOXL2-Containing Media:

MCF7 or SW620 cells were seeded at 50,000 cells per well of an 8-chambered culture slide in HGDMEM (high-glucose Dulbecco's modified Eagle's medium containing 4.5 g/l glucose)+10% FBS, 2 mM L-glutamine, 24 hours prior to being exposed to conditioned medium (CM). 500 uls of fresh conditioned medium from MDA MB 231 cells was added to the chambers containing MCF7 cells. The cells were incubated with the CM for 48-96 hours. Conditioned medium from MCF7 or SW620 cells was used as a negative control. After 48-96 hours incubation with CM, the cells were stained with rhodamine-phalloidin as described above.

LOXL2 Catalytic Activity is Required for EMT-like Change in SW620 Cells Treated with LOXL2 CM

Rat, cynomolgous monkey and human LOXL2 (wild-type and the Y689F mutant) were individually transfected into HEK293 cells in T175 flasks using Lipofectamine 2000 (Invitrogen, Carlsbad, Calif.) according to the manufacturer's instructions. The transfection medium was aspirated four hours after transfection and replaced with 30 ml DMEM+0.5% FBS, and the cells were grown at 37° C., 5% C0₂ for 72 hours. The conditioned medium was collected and concentrated ˜10× in a 10,000 MW cutoff column (Millipore) and filtered through 0.2 um filter (Aerodisk).

Three-Dimensional Collagen I Gels

HFF cells were grown in 2 mg/ml or 3 mg/ml Collagen I gel in 6 well plates at a density of 2×10⁵ cells/well. Wozniak M A & Keely P J (2005) Biological Procedures Online 7(1):144-161. Briefly Collagen 1 (BD Biosciences) was mixed with neutralizing solution (100 mM HEPES in PBS, pH 7.3) and HFF cells in 1 ml of RPMI (Mediatech) containing 10% FBS and 2 mM L-glutamine were added to a well. The plates were incubated at 37° C. for 30 min, then 2 ml/well of RPMI/10% FBS/2 mM glutamine medium was added. Half of the gels were floated in the well by dislodging the gel with a small pipette tip while other half were left attached. Aliquots of the conditioned media were collected on Day 4, Day 7 and Day 8, resolved on a SDS-PAGE gel, and proteins in the gel were transferred to a nitrocellulose membrane by blotting. Membranes were incubated with a mouse anti-LOXL2 monoclonal antibody, then with a HRP-conjugated goat anti-mouse antibody (GE-Healthcare). Signal was developed with a chemiluminescent solution (AlphaInnotech) and analyzed using UVP imaging.

Two-dimensional Polyacrylamide Gel Electrophoresis

Polyacrylamide gels were cast and placed into 6-well plates as previously described (Schlunck et al 2007). Cells were seeded into 6-well plates at 1×10⁵ cells/well and were cultured without media changes for 7-8 days. Subsequently, medium was removed for analysis. Cells on gels were then lysed for protein blot (“Western”) analysis or fixed with 4% paraformaldehyde and stained with rhodamine phalloidin (Invitrogen, Carlsbad, Calif.) according to manufacturer's recommendations.

siRNA Knockdown of Lox and Loxl2 in HFF Cells

siRNA sequences for inhibition of LOXL2 expression were as follows:

(SEQ ID NO: 14)
5′-UAU GCU UUC CGG AAU CUC GAG GGU C-3′
(double-stranded oligo)
(SEQ ID NO: 15)
5′-UGG AGU AAU CGG AUU CUG CAA CCU C-3′
(double-stranded oligo)
(SEQ ID NO: 16)
5′-UCA ACG AAU UGU CAA AUU UGA ACC C-3′
(double-stranded oligo)

siRNA sequence for inhibition of LOX expression was as follows:

(SEQ ID NO: 17)
5′-AUA ACA GCC AGG ACU CAA UCC CUG U-3′
(double-stranded oligo)

Sixty microliters of 20 uM siRNA was mixed into 1 ml of OptiMEM® (final siRNA concentration was 100 nM); and 30 ul of Dharmafect 3 transfection reagent (Thermo Scientific, Chicago, Ill.) was mixed into 1 ml of OptiMEM®. The two mixtures were combined and incubated for 20 minutes at room temperature.

HFF cells were cultured in 10 cm² tissue culture plates until they reached approximately 75% confluency, then they were trypsinized and resuspended in 10 ml of complete medium. Two ml of the transfection mixture (described in the previous paragraph) was added to the cells and the resultant mixture was plated in a 10 cm² culture dish. Cells cultures were harvested after 5 days for measurement of protein levels.

In-Vitro HUVEC Assay

Human umbilical vein endothelial cells (HUVECs) were plated on a feeder layer of fibroblasts and cultured in 24-well plates in Lonza EBM-2 medium (a basal medium developed for normal human endothelial cells in a low-serum environment) supplemented with hEGF, Hydrocortisone, GA-1000 (Gentamicin, Amphotericin-B), FBS (Fetal Bovine Serum) 10 ml (2% final), VEGF, hFGF-B, R3-IGF-1, Ascorbic Acid and heparin. Cells were grown until the cultures demonstrated the earliest stages of tubule formation. At this point (day 1), 0.5 ml of fresh endothelial cell growth medium containing no additions (control), anti-LOXL2 antibody AB0023, or suramin was added to the wells. The plate was then cultured at 37° C. and 5% CO₂. On days 4, 7 and 9 the medium was removed from all wells and carefully replaced with 0.5 ml of fresh medium containing the additions listed above. On day 11, the plates were fixed and tubules were assayed for CD31 expression as follows. Wells were washed with 1 ml PBS and fixed with 1 ml ice cold 70% ethanol for 30 minutes at room temperature. Cells were then incubated, for 60 minutes at 37° C., with 0.5 ml mouse anti-human CD31 antibody diluted 1:400 in PBS containing 1% BSA. Wells were then washed three times with 1 ml PBS, followed by incubation for 60 minutes at 37° C. with 0.5 ml alkaline phosphatase-conjugated goat anti-mouse IgG secondary antibody diluted 1:500 in PBS containing 1% BSA. Wells were washed a further three times with 1 ml PBS prior to incubation for 10 minutes at 37° C. with 0.5 ml freshly prepared and filtered BCIP/NBT substrate. Wells were then carefully washed three times with 1 ml distilled water and left to air dry overnight.

Digital images of each well were taken using a Nikon Coolpix camera on a Leica inverted microscope at 10× magnification. Four random fields per well were imaged, producing 96 images in total. Images were then converted to BMP files and imported into an image analysis package to measure number of vessel branches, number of vessels and total vessel length. These measurements were performed by thresholding the image so as to detect only CD31-stained vessels, which the software then skeletonises to produce single pixel width vessels, from which it is able to measure individual vessel length, total vessel length and number of vessel branch points. The data is then exported to Excel to calculate mean and standard deviation for each data set. Basic statistics were carried out using a one way ANOVA.

### Example 7

### Xenograft Model of Metastasis and Primary Tumorigenesis

To provide growing tumor tissue stock for subsequent orthotopic implantation, five- to six-week-old nude mice (NCr nu/nu) were injected subcutaneously with 2×10⁶ MDA-MB-435-GFP cells (Anticancer, Inc., San Diego, Calif.) on the right flank. For this purpose, cultures of MDA-MB-435-GFP cells were harvested and dissociated by trypsinization, washed three times with cold serum-containing medium, and then kept on ice until injection. Cells were injected into the subcutaneous space of the flank of the animal in a total volume of 0.1 ml, within 30 min of harvesting. The nude mice were sacrificed to harvest tumor tissue 4 to 6 weeks after tumor cell injection for surgical orthotopic implantation (SOI) of tumor fragments.

Tumor pieces (˜1 mm³), extracted from subcutaneously-growing GFP-expressing breast tumors, were implanted by surgical orthotopic implantation (SOI) on the breast of female nude mice (NCr nu/nu). Treatments with AB0023 (anti-LOXL2 monoclonal antibody), M64 (anti-LOX monoclonal antibody) and vehicle (all via intraperitoneal injection) and with Taxotere (by intravenous injection) were initiated when the average primary tumor volume reached 75 mm³. Mice were administered the antibodies at a dose of 30 mg/kg twice a week for 28 days and Taxotere, at 10 mg/kg, was administered once a week for 3 weeks.

Body weight and tumor size were recorded weekly. At the conclusion of the study, mice were sacrificed by cervical dislocation after being anesthetized with carbon dioxide. Primary breast tumors were imaged, harvested, cut in half symmetrically and snap-frozen for histological and immunohistochemical analyses.

### Example 8

### CCl₄-Induced Liver Fibrosis

Male BALB/c mice (10-12 weeks old) were obtained from Aragen Biosciences (Morgan Hill, Calif.). Mice were distributed into 4 groups. Mice in 3 of the groups were injected with CCl₄ (Sigma-Aldrich, St. Louis, Mo.), and mice in the remaining group were injected with saline.

CCl₄ was intraperitoneally administered to mice at 1 ml/kg body weight (CCl₄: mineral oil in 1:1 (v/v) ratio) twice weekly for 4 weeks. In the control group, 0.9% saline (saline:mineral oil in 1:1 (v/v) ratio) was administered intraperitoneally using the same dosing regimen.

In the CCl₄-treated groups, the first group was treated with AB0023 (diluted in PBS/0.01% Tween-20), the second group was treated with pep4 M64 (diluted in 10 ml-histidine buffer) and the third group was treated with vehicle (PBST). Antibodies and vehicle were injected intraperioneally at a dose of 30 mg/kg twice a week. The treatment started a day prior to the first administration of CCl₄ and continued until the end of the study. The study was terminated after 4 weeks of CCl₄ and antibody administration. Mice were euthanized and sacrificed humanely and the livers were harvested 96 hours after completion of dosing. The livers were snap-frozen for histological and immunohistochemical analyses.

### Example 9

### In-Vivo Matrigel Plug Angiogensis Assay

Athymic female Ncr:Nu/Nu mice were injected subcutaneously in the flank with 0.5 ml high-concentration Matrigel (BD Biosciences, San Jose, Calif.) supplemented with 100 ng/ml FGF and 60 U heparin. Matrigel injections were conducted one week after initiation of treatments with antibodies). Antibodies (or PBST, as a control) were administered by intraperitoneal injection of 30 mg/kg twice weekly. Matrigel plugs were harvested 10 days after implantation by excising the plug together with attached skin, and were fixed in 10% neutral buffered formalin and embedded in paraffin. 5 um sections were cut and stained with hematoxylin and eosin, anti-CD31 or anti-CD34 antibodies to assess degree of vessel formation.

### Example 10

### Immunohistochemistry

The solutions used for the immunohistochemistry (IHC) protocols were obtained from Biocare Medical (Concord, Calif.) unless otherwise stated. All procedures were performed at room temperature. Slides were fixed with 4% PFA for 10 minutes and were subsequently treated with Peroxidazed-1 (Biocare Medical, Concord, Calif.) for 5 minutes. Then, the slides were background blocked with SNIPER (Biocare Medical, Concord, Calif.) for 10 minutes. Primary antibody (2-5 ug/ml final concentration) was diluted in Da Vinci Green Universal Diluent and applied to slides for 30 minutes. Slides were then rinsed in PBS-Tween-20. The Mach2 polymer kit was used for antigen detection by adding rabbit probe for 30 minutes. DAB chromagen was added to the slides for 3-5 minutes, then slides were rinsed once with deionized water. The slides were then counter-stained with hematoxylin, followed by dehydration with graded alcohol. The slides were mounted with entellan mounting medium (Electron Microscopy Sciences, Hatfield, Pa.).

### Example 11

### Quantitative Analysis of IHC Images

Liver Fibrosis

Ten fields (or areas or lobes) for each treatment regimen were randomly selected and stained with Sirius Red. The area used for scoring was 1.7 mm×1.3 mm and contained at least 8 portal triads.

Triads and areas of complete bridging fibrosis were counted. The number of areas of complete bridging fibrosis was divided by total number of triad areas and the percentage of complete bridging fibrosis was obtained from each field. The percentages from 10 fields (per treatment) were averaged and standard error was calculated.

Fibroblast Activation by Staining for aSMA Expression

For each treatment, five fields were selected and stained with an anti-alpha-smooth muscle actin (aSMA) antibody. aSMA-positive signal in the porto-portal region (threshold area %) was analyzed by Metamorph (Molecular Devices, Downingtown, Pa.). aSMA-positive signal in sections from animals undergoing AB0023-treatment was compared to signal obtained in sections from animals that had been treated with vehicle (PBS).

### Example 12

### RT-PCR Analysis

Total RNA Isolation and Quantitative Real-Time PCR

RNA was extracted from pieces of frozen tissue using a RNeasy Mini Kit (Qiagen, Valencia, Calif.) according to the manufacturer's instructions. Briefly, tissues were homogenized in RLT lysis buffer with a Polytron hand-held electric homogenizer, and eluted with nuclease-free water (Ambion, Austin, Tex.). Residual genomic DNA contamination was removed using recombinant DNase I (Ambion, Austin, Tex.). One hundred nanograms of total RNA per reaction was used for reverse transcriptase-mediated cDNA synthesis and subsequent PCR with the BrilliantII qPCR one-step core reagent kit (Agilent, Santa Clara, Calif.). Reactions were conducted in duplicate on a Mx3000P instrument (Agilent, Santa Clara, Calif.). Gene expression was analyzed by real-time PCR (TaqMan®), using species-specific primer and probe sets designed by Beacon Designer software for human (h) and mouse (m):

hLOX:
Forward

| | |
|---|---|
| 5′ CTTGACTGGGGAAGGGTCTG 3′, | (SEQ ID NO: 18) |
| Reverse | |
| 5′ AAAACGGGGCTCAAATCACG 3′, | (SEQ ID NO: 19) |
| Probe | |
| 5′ ATCCCACCCTTGGCATTGCTTGGT 3′ | (SEQ ID NO: 20) |

hLOXL1:

| | |
|---|---|
| Forward | |
| 5′ AGCAGACTTCCTCCCCAACC 3′, | (SEQ ID NO: 21) |
| Reverse | |
| 5′ CAGTAGGTCGTAGTGGCTGAAC 3′ | (SEQ ID NO: 22) |
| Probe | |
| 5′ CACGGCACACCTGGGAGTGGCAC 3′ | (SEQ ID NO: 23) |

hLOXL2:

| | |
|---|---|
| Forward | |
| 5′ GGGGTTTGTCCACAGAGCTG 3′, | (SEQ ID NO: 24) |
| Reverse | |
| 5′ ACGTGTCACTGGAGAAGAGC 3′, | (SEQ ID NO: 25) |
| Probe | |
| 5′ TGGAGCAGCACCAAGAGCCAGTCT 3′ | (SEQ ID NO: 26) |

hLOXL3:

| | |
|---|---|
| Forward | |
| 5′ GTGTGCGACAAAGGCTGGAG 3′, | (SEQ ID NO: 27) |
| Reverse | |
| 5′ CCGCGTTGACCCTCTTTTCG 3′, | (SEQ ID NO: 28) |
| Probe | |
| 5′ AAGCCCAGCATCCCGCAGACCAC 3′ | (SEQ ID NO: 29) |

hLOXL4:

| | |
|---|---|
| Forward | |
| 5′ CTTACCACACACATGGGTGTTTC 3′, | (SEQ ID NO: 30) |
| Reverse | |
| 5′ TCAAGCACTCCGTAACTGTTGG 3′, | (SEQ ID NO: 31) |
| Probe | |
| 5′ CCTTGGAAGCACAGACCTCGGGCA 3′ | (SEQ ID NO: 32) |

hACTA2: (alpha-smooth muscle actin)

| | |
|---|---|
| Forward | |
| 5′ CTATCCAGGCGGTGCTGTC 3′, | (SEQ ID NO: 33) |
| Reverse | |
| 5′ ATGATGGCATGGGGCAAGG 3′, | (SEQ ID NO: 34) |
| Probe | |
| 5′ CCTCTGGACGCACAACTGGCATCG 3′ | (SEQ ID NO: 35) |

hFN1: (fibronectin)

| | |
|---|---|
| Forward | |
| 5′ TGGGAGTTTCCTGAGGGTTTTC 3′, | (SEQ ID NO: 36) |
| Reverse | |
| 5′ GCATCTTGGTTGGCTGCATATG 3′, | (SEQ ID NO: 37) |
| Probe | |
| 5′ AGGGCTGCACATTGCCTGTTCTGC 3′ | (SEQ ID NO: 38) |

hVIM: (vimentin)

| | |
|---|---|
| Forward | |
| 5′ CAGGCAAAGCAGGAGTCCAC 3′, | (SEQ ID NO: 39) |
| Reverse | |
| 5′ CTTCAACGGCAAAGTTCTCTTCC 3′, | (SEQ ID NO: 40) |
| Probe | |
| 5′ ACCGGAGACAGGTGCAGTCCCTCA 3′ | (SEQ ID NO: 41) |

hSNAI1: (Snail)

| | |
|---|---|
| Forward | |
| 5′ TCAAGATGCACATCCGAAGCC 3′, | (SEQ ID NO: 42) |
| Reverse | |
| 5′ CAGTGGGGACAGGAGAAGGG 3′, | (SEQ ID NO: 43) |
| Probe | |
| 5′ CCTGCGTCTGCGGAACCTGCGG 3′ | (SEQ ID NO: 44) |

hCOL1A1: (Type I Collagen)

| | |
|---|---|
| Forward | |
| 5′ ACAGAACGGCCTCAGGTACC 3′, | (SEQ ID NO: 45) |
| Reverse | |
| 5′ TTCTTGGTCTCGTCACAGATCAC 3′, | (SEQ ID NO: 46) |
| Probe | |
| 5′ CGTGTGGAAACCCGAGCCCTGCC 3′ | (SEQ ID NO: 47) |

hRPL19: (Ribosomal protein L19)

| | |
|---|---|
| Forward | |
| 5′ CCGGCTGCTCAGAAGATAC 3′, | (SEQ ID NO: 48) |
| Reverse | |
| 5′ TTCAGGTACAGGCTGTGATACAT 3′, | (SEQ ID NO: 49) |
| Probe | |
| 5′ TGGCGATCGATCTTCTTAGATTCACG 3′ | (SEQ ID NO: 50) |

mLOX:

| | |
|---|---|
| Forward | |
| 5′ CAAGAGGGAAGCAGAGCCTTC 3′, | (SEQ ID NO: 51) |
| Reverse | |
| 5′ GCACCTTCTGAATGTAAGAGTCTC 3′, | (SEQ ID NO: 52) |
| Probe | |
| 5′ ACCAAGGAGCACGCACCACAACGA 3′ | (SEQ ID NO: 53) |

mLOXL1:

| | |
|---|---|
| Forward | |
| 5′ GGCCTTCGCCACCACCTATC 3′, | (SEQ ID NO: 54) |
| Reverse | |
| 5′ GTAGTACACGTAGCCCTGTTCG 3′, | (SEQ ID NO: 55) |
| Probe | |
| 5′ CCAGCCATCCTCCTACCCGCAGCA 3′ | (SEQ ID NO: 56) |

mLOXL2:

| | |
|---|---|
| Forward | |
| 5′ GCTATGTAGAGGCCAAGTCCTG 3′, | (SEQ ID NO: 57) |
| Reverse | |
| 5′ CAGTGACACCCCAGCCATTG 3′, | (SEQ ID NO: 58) |
| Probe | |
| 5′ TCCTCCTACGGTCCAGGCGAAGGC 3′ | (SEQ ID NO: 59) |

mLOXL3:

| | |
|---|---|
| Forward | |
| 5′ AACGGCAAGCTGTCTGGAAG 3′, | (SEQ ID NO: 60) |
| Reverse | |
| 5′ AGCCAACATTGACCTAGCACTG 3′, | (SEQ ID NO: 61) |
| Probe | |
| 5′ TCCCGCCCATTCCCACCCATCTCG 3′ | (SEQ ID NO: 62) |

mLOXL4:

| | |
|---|---|
| Forward | |
| 5′ CAAGACAGGTCCAGTAGAGTTAGG 3′, | (SEQ ID NO: 63) |
| Reverse | |
| 5′ AGGTCTTATACCACCTGAGCAAG 3′, | (SEQ ID NO: 64) |
| Probe | |
| 5′ ACAGAGCACAGCCGCCTCACTGGA 3′ | (SEQ ID NO: 65) |

mACTA2: : (alpha-smooth muscle actin)

| | |
|---|---|
| Forward | |
| 5′ TCTGCCTCTAGCACACAACTG 3′, | (SEQ ID NO: 66) |
| Reverse | |
| 5′ AAACCACGAGTAACAAATCAAAGC 3′, | (SEQ ID NO: 67) |
| Probe | |
| 5′ TGTGGATCAGCGCCTCCAGTTCCT 3′ | (SEQ ID NO: 68) |

mFN1: (fibronectin)

| | |
|---|---|
| Forward | |
| 5′ CACCTCTGCTTTCTTTTGCCATC 3′, | (SEQ ID NO: 69) |
| Reverse | |
| 5′ CTGTGGGAGGGGTGTTTGAAC 3′, | (SEQ ID NO: 70) |
| Probe | |
| 5′ TGCAGCACTGTCAGGACATGGCCT 3′ | (SEQ ID NO: 71) |

mVIM: (vimentin)

| | |
|---|---|
| Forward | |
| 5′ CGCCCTCATTCCCTTGTTGC 3′, | (SEQ ID NO: 72) |
| Reverse | |
| 5′ GGAGGACGAGGACACAGACC 3′, | (SEQ ID NO: 73) |
| Probe | |
| 5′ TTCCAGCCGCAGCAAGCCAGCC 3′ | (SEQ ID NO: 74) |

mCOL1A1: (Type I Collagen)

| | |
|---|---|
| Forward | |
| 5′ CGGCTGTGTGCGATGACG 3′, | (SEQ ID NO: 75) |
| Reverse | |
| 5′ ACGTATTCTTCCGGGCAGAAAG 3′, | (SEQ ID NO: 76) |
| Probe | |
| 5′ CAGCACTCGCCCTCCCGTCTTTGG 3′ | (SEQ ID NO: 77) |

mRPL19: (Ribosomal protein L19)

| | |
|---|---|
| Forward | |
| 5′ AGAAGGTGACCTGGATGAGAA 3′, | (SEQ ID NO: 78) |
| Reverse | |
| 5′ TGATACATATGGCGGTCAATCT 3′, | (SEQ ID NO: 79) |
| Probe | |
| 5′ CTTCTCAGGAGATACCGGGAATCCAAG 3′ | (SEQ ID NO: 80) |

Average fold-changes in transcript levels were calculated by differences in threshold cycles (Cₜ) between tumor and normal samples. Expression levels were normalized to those of the RPL19 gene.

### Example 13

### LOXL2 is Strongly Expressed by the Stroma of Diverse Tumor Types and by Pathogenic Cells in Liver Fibrosis

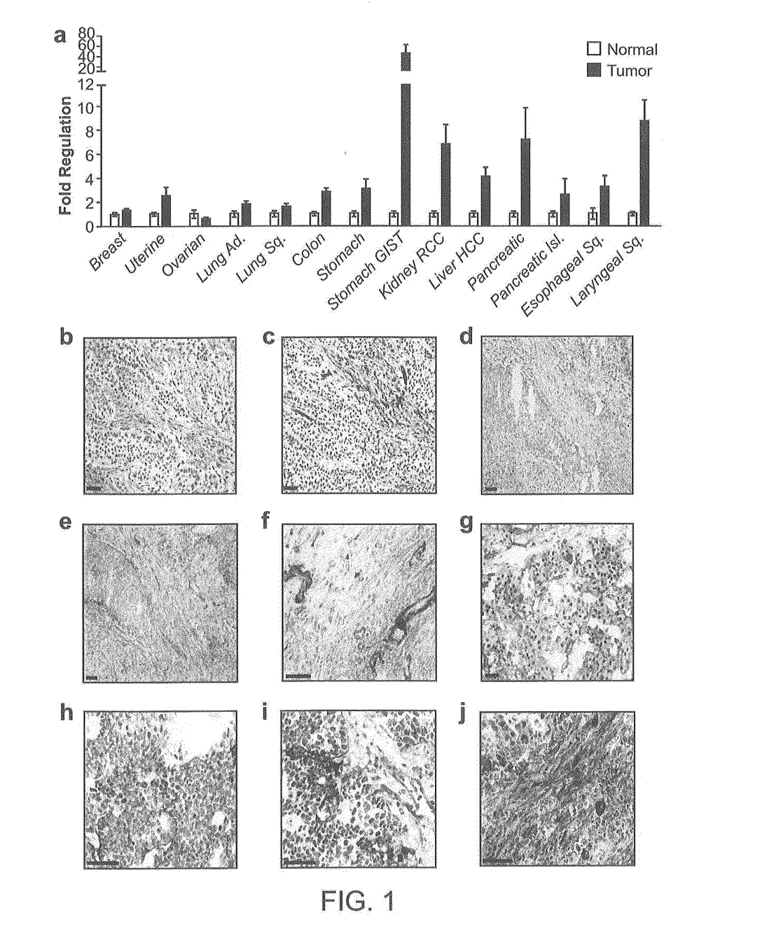

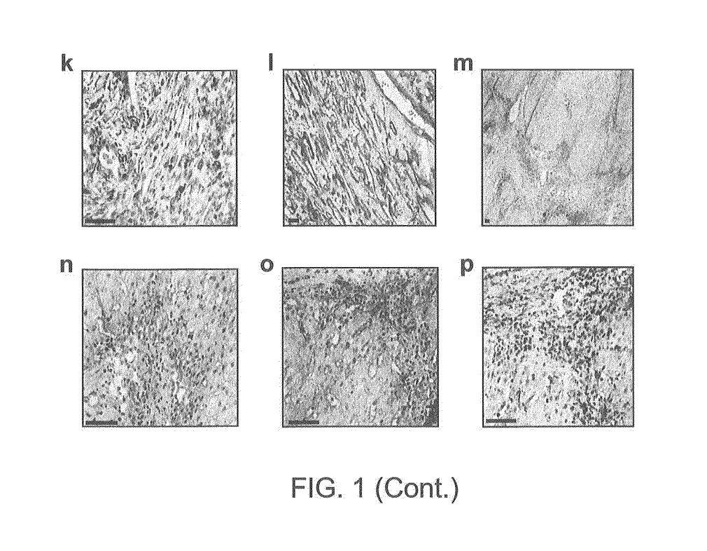

Analysis of LOXL2 transcript in tumors revealed elevated expression in most major solid tumors when compared to non-neoplastic tissues (summarized in FIG. 1, Panel A). In several tumor types, LOXL2 transcript showed a trend of increased expression with increasing stage or grade (such as colon, pancreatic, uterine, renal cell, stomach and head and neck cancers, FIG. 7, panels A, B, C, D, E, F; also elevated transcript in grade III lung adenocarcinoma (not shown)). The distribution and localization of LOXL2 protein in tumors was further investigated by immunohistochemistry using a LOXL2-specific polyclonal antibody (FIG. 7, panel G) and minimally-processed fresh-frozen tissues. In addition to some cytoplasmic signal, LOXL2 was abundantly secreted in tumors and often associated with regions of collagenous matrix (FIG. 1, Panels B, C, D, E, F; FIG. 7, panels H, I, J, K, M, O). A similar pattern of expression was observed across diverse solid tumor types, with LOXL2 expression by stromal fibroblasts and vasculature, and by some regions of tumor cells (FIG. 1, Panels C, E, F, G, H, I, J; FIG. 7, panels H-O). FIG. 1, Panel k shows LOX expression. The stromal fibroblasts expressing LOXL2 were αSMA positive (not shown). Significant secreted LOXL2 signal was detected at active disease interfaces such as the tumor-stroma boundary (FIG. 1, Panels E, F, G; FIG. 7, Panel H), and strong LOXL2 signal was associated with glomeruloid microvascular structures indicative of tumor-associated angiogenesis (FIG. 1, Panels F, I). LOXL2 was also strongly expressed in highly angiogenic tumors such as clear cell renal cell carcinomas (FIG. 1, Panel L). In comparison, little LOXL2 protein was detected in most non-neoplastic tissues and major organs such as the heart, liver and lungs (FIG. 7, Panels P, R, S; summarized in Table 1 of FIG. 7). Some signal was observed in reproductive organs such as ovary and uterus, consistent with previous reports, as well as reticular fibers in spleen (examples in FIG. 7, Panels U, V), and some regions of non-neoplastic kidney. Despite strong expression on tumor-associated vasculature, little LOXL2 protein was detected in the vasculature of healthy tissues (FIG. 7, panels P, Q, S, T). The differential expression, secretion and association with active disease support the targeting of LOXL2 in oncology. While tumor cell expression of LOXL2 has been reported previously (primarily cytoplasmic), this analysis revealed widespread expression by tumor-associated stromal cells such as TAFs and neovasculature. This pattern of localization for LOXL2 was conserved among solid tumors of different origins.

In comparison, the predominant pattern of localization detected for LOX protein in neoplastic tissue was cytoplasmic staining of fibroblasts and endothelial cells, and some tumor cells, with less evidence for secretion and less association with the collagenous matrix in tumors. This pattern of expression was also conserved among different tumor types (FIG. 1, Panel K, FIG. 7, panels L, N). In contrast to LOXL2, high levels of LOX protein were detected in normal tissues such as artery and vascular and non-vascular smooth muscle (FIG. 7, Panels W, X, Y, Z). Significant LOX expression in artery is consistent with literature describing bovine aorta as a primary source of cleaved, enzymatically active LOX protein.

Expression of LOXL2 and LOX was also evaluated in fibrotic liver. LOXL2 was highly expressed and secreted at the disease interface comprised of fibroblasts, hepatocytes, blood vessels and inflammatory cells (FIG. 1, Panels M, N, O). LOX protein was also detected, but with a predominantly cytoplasmic cellular localization in fibroblasts (FIG. 1, Panel P). Despite the different etiologies for these diseases, similar patterns of expression and localization for LOX and LOXL2 were observed for both tumors and active fibrotic liver.

### Example 14

### Secreted LOXL2 Promotes Remodeling and Invasion of Tumor Cells in Vitro

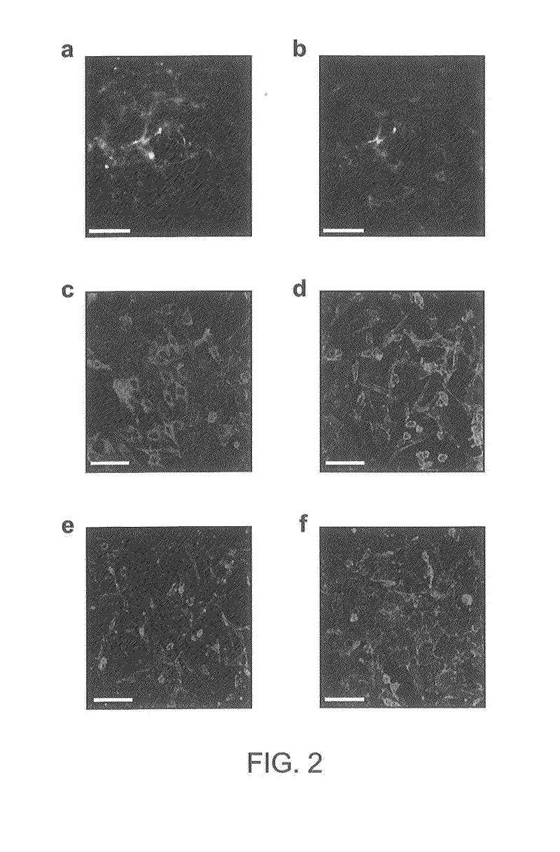

LOXL2 was expressed by a number of different tumor cell lines under normoxic conditions (FIG. 8, panels A, B). LOXL2 protein was detected in conditioned media as both full-length (˜80 kDa) and cleaved proteins (˜55 KDa). Analysis of purified LOXL2 protein revealed that both these forms of LOXL2 were enzymatically active and were inhibited by BAPN in vitro (FIG. 8, panels C, D), contrary to previous reports. Use of immunoflourescence and a LOXL2-specific monoclonal antibody (AB0023) indicated that LOXL2 was co-localized with its substrate collagen I, in the extracellular matrix of tumor cells, consistent with the results obtained for tumor tissues (FIG. 2. Panels A, B; FIG. 8, panels E, F, G). In comparison, while secreted processed LOX was present in conditioned media from an osteoblast cell line (FIG. 8, panel H), LOX was not detected reproducibly in the conditioned media isolated from tumor cell lines or fibroblasts under normoxic or hypoxic conditions (FIG. 8, panels I, J) but was found instead in the cell pellet fraction.

LOXL2 has been described as playing a role in the epithelial-to-mesenchymal transition (EMT) via direct interaction with the EMT-associated transcription factor SNAIL. Depletion of LOXL2 using shRNA knockdown of LOXL2 in breast tumor cell line MDA-MB-231, which expresses all 5 lysyl oxidase-type enzymes, resulted in remodeling of the actin cytoskeleton to produce a more epithelial phenotype with an associated reduction in actin stress fibers (visualized by phalloidin staining, FIG. 8, panels K, L). However, a full mesenchymal-to-epithelial transition (MET)-like change was not observed as a result of LOXL2 inhibition, since the cells remained negative for E-cadherin.

We explored the role of extracellular LOXL2 in remodeling of tumor cells by treatment of MCF7 cells (which express little LOXL2 under normoxic conditions) with conditioned media from MDA-MB231 or Hs-578t tumor cells, which contains endogenously secreted LOXL2. The conditioned media was treated with either an IgG control antibody or AB0023, an inhibitory LOXL2 monoclonal antibody. AB0023 binds to the SRCR3-4 region of LOXL2, demonstrates no cross-reactivity with other lysyl oxidase-type enzymes (FIG. 8, panels M, N), and inhibits the lysyl oxidase enzymatic activity of LOXL2 (FIG. 8, panel O). AB0023 binds human and mouse LOXL2 with similar affinity (FIG. 8, panel P). The LOXL2-containing conditioned media induced remodeling of the actin cytoskeleton resulting in elongated cell morphology and increased actin stress fibers, and this remodeling was abrogated by addition of AB0023 (FIG. 2, Panels C, D, E, F). However, this change in phenotype was not inhibited by pre-incubation of the conditioned media with BAPN, even at high concentrations (2 mM, data not shown). Purified, enzymatically active LOXL2 alone was not capable of inducing the cellular remodeling, suggesting that the LOXL2 secreted by cells induces these changes in concert with other protein/s. To further investigate the domains required for phenotypic remodeling by secreted LOXL2, truncated and mutated versions of the protein were expressed and evaluated for their ability to induce this change, including the N-terminal SRCR domains and a secreted but enzymatically-inactive variant of LOXL2 generated by mutation of the lysine residue required for LTQ formation in the enzymatic domain. When expressed in conditioned media, both the SRCR domains alone (data not shown) and enzmatically-inactive LOXL2 were incapable of inducing remodeling (FIG. 8, panels Q, R, S, T), indicating that both the enzymatic domain and enzymatic activity are required for this process. Overall, these data support a role for secreted, enzymatically-active LOXL2 in remodeling the actin cytoskeleton of tumor cells toward a more mesenchymal phenotype.

### Example 15

### LOXL2 Promotes Fibroblast Activation In Vitro and In Vivo

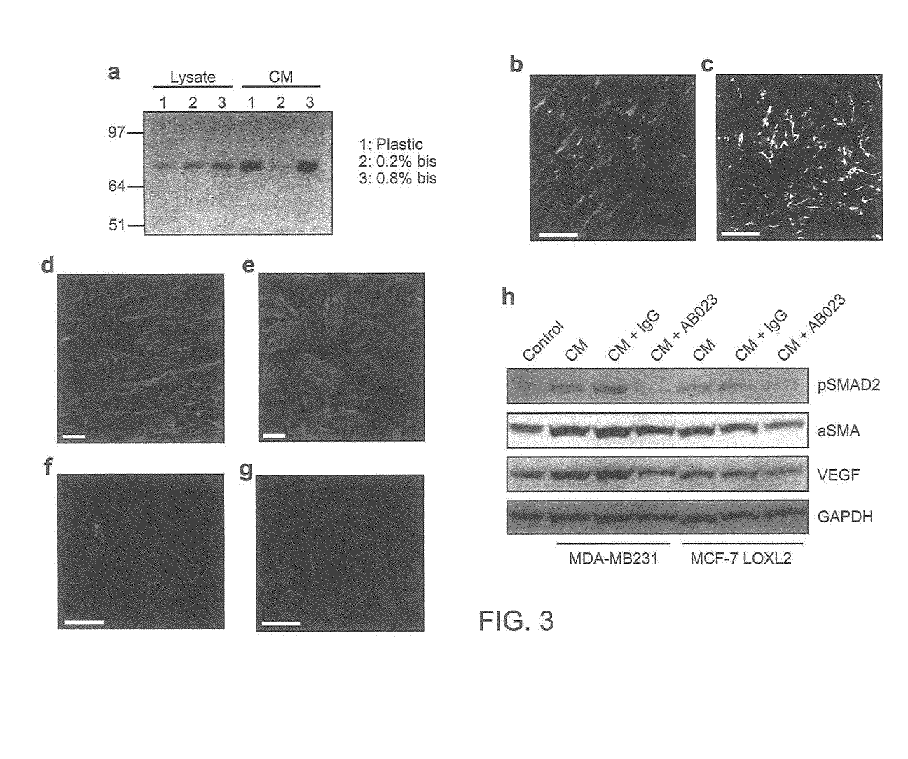

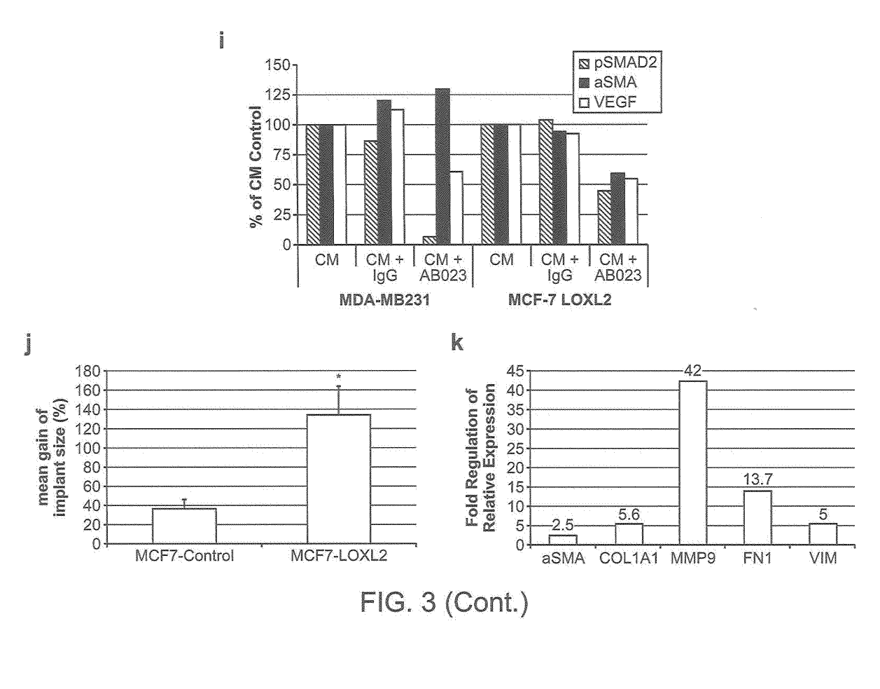

To investigate the regulation of secretion of LOXL2 by fibroblasts, in vitro models of variable tension were established using bis-acrylamide cross-linked gels coated with a collagen matrix, and floating or attached collagen gels. At low tension (0.2% bis, and floating collagen gels), LOXL2 protein was detected intracellularly with very limited secreted protein apparent in conditioned media. At higher tension (0.8% bis, attached collagen gels, and standard tissue-culture plates), LOXL2 was abundantly secreted by fibroblasts (FIG. 3, Panel A, FIG. 9, panel A). These data suggest that LOXL2 secretion could be induced by changes in local tension (for example, associated with inflammation or matrix production or crosslinking). Significant levels of secreted LOX were not detected from HFF cells grown on either the 0.2 or 0.8% bis-acrylamide collagen gels or on floating or attached collagen gels, but only for HFF cells grown on tissue-culture treated plastic.

Given the strong expression of LOXL2 by TAFs in human tumors and the effects of LOXL2 in tumor cell remodeling, a role for LOXL2 in fibroblast morphology was examined using siRNA knockdown. Depletion of LOXL2 in HFF resulted in reduced intercellular organization when compared to control-transfected cells. siLOXL2 cells still secreted collagen I, but the collagen was disorganized and lacked the fibrillar structural organization apparent in control transfected cells (FIG. 3, Panels B, C; FIG. 9, panel B). Staining of siLOXL2 knockdown cells using phalloidin revealed dramatic alterations to the actin cytoskeleton: cells became less elongated and more rounded, with a reduction in either central or peripheral actin stress fibers (FIG. 3. Panels D, E). Consistent with this change in phenotype, fibroblasts grown under low-tension conditions (0.2% bis), which secrete little LOXL2, demonstrated a more rounded, epithelioid structure with reduced actin stress fibers, whereas cells grown under higher tension (0.8% bis), which secrete significantly more LOXL2, adopted a more elongated, fibroblastoid phenotype (FIG. 3, Panels F, G). These findings support a role for LOXL2 in maintenance of an activated fibroblastic morphology and intercellular organization via the collagenous extracellular matrix.

The involvement of LOXL2 with pathways associated with fibroblast activation, fibrosis and desmoplasia was examined. No significant effects on AKT phosphorylation were observed upon treatment of cells with PDGF-BB in fibroblasts or tumor cell lines in the presence or absence of AB0023 over 10-60 minute time periods (not shown). Investigation of TGFb signaling revealed evidence of modest inhibition (about 10-20%) of SMAD2 phosphorylation by AB0023 in a 10-60 minute time frame. However, more dramatic effects were apparent upon treatment of HFF cells with LOXL2-containing conditioned media from tumor cell lines by transwell co-culture for a more prolonged period of time. After 72 hours, co-culture in the presence of AB0023 resulted in a relative decrease in pSMAD2 phosphporylation of 56-94% (FIG. 3<Panels H, I). A reduction of 41% in α-SMA levels was also observed. Evaluation of VEGF protein expression, a characteristic of tumor-associated fibroblasts, revealed a 39-46% reduction in the presence of AB0023. Overall, these data suggest that LOXL2 does not act by directly potentiating signaling pathways driven by growth factors, as more profound effects might be expected in experiments performed over short incubation times. Rather, these results may indicate that LOXL2 can mediate TGFb signaling and associated fibroblast activation by its activity on the extracellular matrix. Activation of TGFb signaling from the latent complex in the extracellular matrix has been shown to be induced by increases in tension. These findings are therefore consistent with the modulation of signaling via LOXL2-induced changes in the extracellular matrix, either directly or possibly through integrin-mediated sensing of cross-linked fibrillar collagen.

The consequences of LOXL2 expression were evaluated in vivo by comparing tumor formation of MCF7 control cells with MCF7 cells stably transfected with an expression vector encoding LOXL2 (MCF7-LOXL2). The proliferation rate of the transfected cells in vitro was less than that observed for MCF7-control cells. However, upon generation of tumors in the sub-renal capsule of nu/nu mice, MCF7-LOXL2 cells yielded larger tumors (3.5× increased volume) compared to control MCF7 cells (FIG. 3, Panel J). Analysis of the stromal components of these tumors using qRT-PCR with mouse-specific primers indicated that, compared to controls, MCF7-LOXL2 cells had induced activation of the stroma with increases in aSMA, collagen I, vimentin, MMP9 and fibronectin transcripts (FIG. 3, Panel K). These results support a role for LOXL2 in activation and remodeling of tumor-associated stroma in vivo.

Overall, these results suggest that disruption of LOXL2 can lead to perturbation of the interaction between cells and their environment, likely through disruption of collagen/matrix mediated signaling, resulting in disruption of both intracellular organization of the actin cytoskeleton and intercellular organization of fibroblasts. These data also indicated that LOXL2 could play an auto-stimulatory role in maintenance of a more highly activated state, and thus be important for the ongoing activation of disease-associated fibroblasts such as TAFs and myofibroblasts.

### Example 16

### Anti-LOXL2 Antibody AB0023 Inhibits Angiogenesis In Vitro and In Vivo

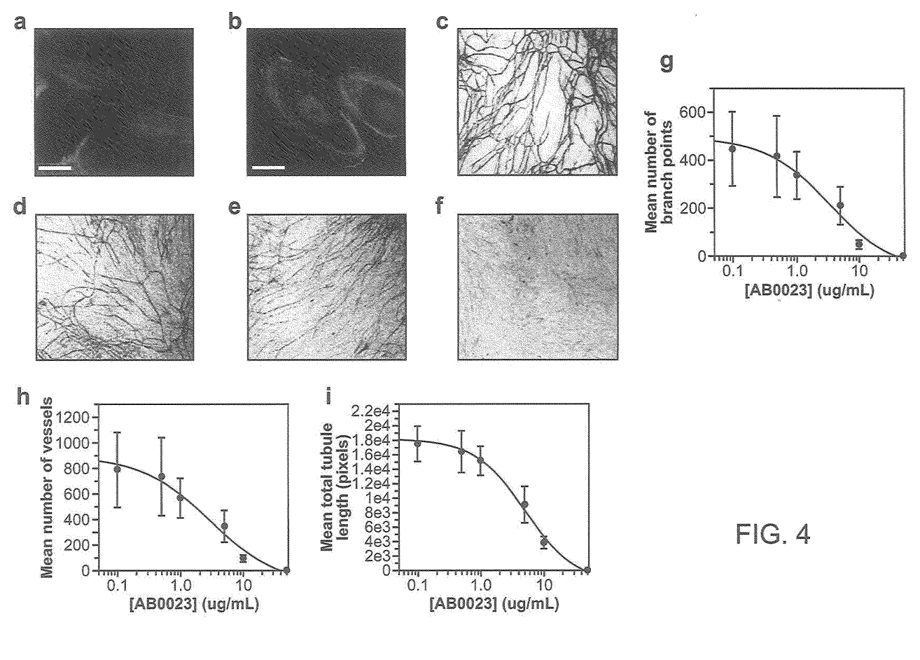

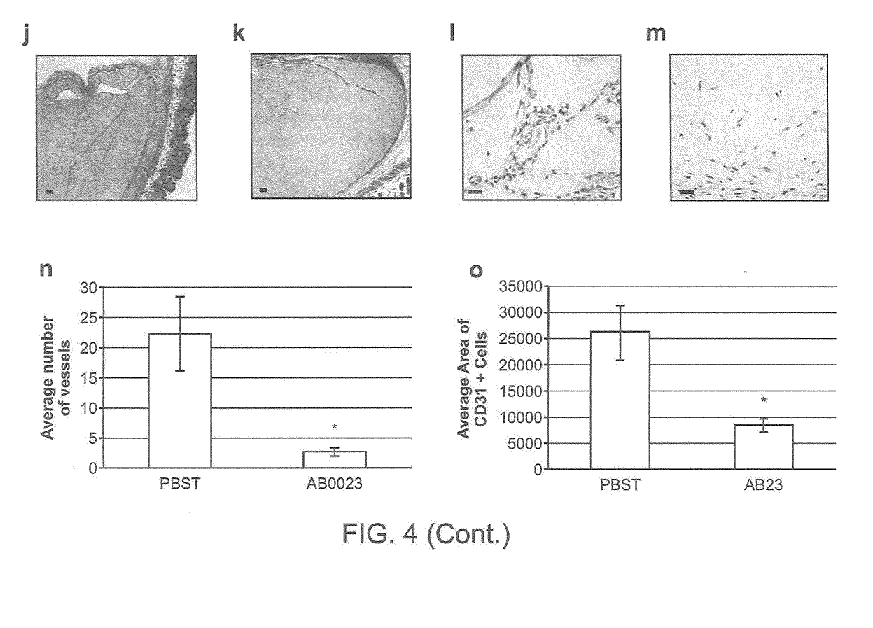

Analysis of human tumors revealed striking expression of LOXL2 by endothelial cells of glomeruloid vessels and other neovasculature. LOXL2 is known to be expressed by cultured primary endothelial cell and has been described as important for vascular elastogenesis in this context. HUVEC cells were depleted for LOXL2 using siRNA knockdown and examined for changes in morphology. Compared to control-transfected cells, siLOXL2 HUVEC cells demonstrated a reduction in actin stress fibers (FIG. 4, Panels A, B), similar to the effects of LOXL2 inhibition on fibroblasts and tumor cells, described above.

To further assess the role of secreted LOXL2, the ability of anti-LOXL2 antibody AB0023 to inhibit angiogenesis was investigated using an in vitro HUVEC tube formation assay. AB0023 inhibited vessel branching, vessel length and the total number of vessels formed in a dose-dependent manner with complete inhibition of all processes at 50 ug/ml (FIG. 4, Panels C, D, E, F, G, H, I). The calculated IC50 for inhibition of each of these processes by AB0023 (FIG. 4, Panels G, H, I; 22.2 nM, 19.9 nM, 33.2 nM, respectively) is consistent with the apparent IC50 observed in vitro for inhibition of purified LOXL2 enzymatic activity by AB0023 (˜30 nM).

The ability of AB0023 to inhibit angiogenesis in vivo was assessed using Matrigel plugs, containing bFGF, inserted in the flank of Balb/C mice. Plugs isolated from vehicle-treated animals contained evidence of invading and branching vasculature comprised of CD31-positive cells (FIG. 4, Panels J, L), whereas plugs isolated from animals treated with AB0023 by intraperitoneal injection displayed limited evidence of vasculature and far fewer CD31-positive cells (FIG. 4, Panels K, M). LOXL2 expression by infiltrating endothelial cells was confirmed by IHC (FIG. 10, panels A, B). Quantitative analysis of the average number of vessels from independent plugs yielded a ˜7-fold reduction for AB0023 treated animals (p=0.0319; FIG. 4, Panel N). A separate analysis quantifying CD31 positive cells in the matrigel plugs also revealed a significant decrease in AB0023-treated animals (p=0.0168; FIG. 4, Panel O). These results indicate that secreted LOXL2 plays an important role in multiple aspects of angiogenesis, and that angiogenesis is inhibited directly by AB0023 both in vitro and in vivo.

### Example 17

### Inhibition of LOXL2 Provides Therapeutic Benefits In Vivo in Both Primary Tumor and Metastatic Xenograft Models of Cancer

The therapeutic consequences of inhibiting either LOXL2 or LOX were assessed in a model of disseminated bone metastasis. Specific antibodies targeting LOXL2 or LOX were used to treat mice injected in the left ventricle with ˜1 million labeled MDA-MB-231 cells. The breast tumor cell line MDA-MB-231 has been widely used as a model to study LOX, and expresses all 5 lysyl oxidase-type enzymes (FIG. 11, panel A). The ability of LOXL2 inhibitory antibody AB0023 to reduce tumor burden was compared to LOX-specific antibody M64, which is a monoclonal antibody targeting the same peptide sequence in the LOX enzymatic domain previously described as generating an inhibitory polyclonal antiserum. After 28 days, a significant reduction in tumor cell burden in the femurs and in total ventral bone was observed for the anti-LOXL2 AB0023-treated tumors (femurs 127-fold by median value, p=0.0021; total ventral bone 28-fold by median value, p=0.0197; FIG. 5, Panels A, B), but not for anti-LOX antibody M64 treated tumors (p=0.5262 and 0.5153 respectively; FIG. 5, Panels A, B; high-dose taxotere (20 mg/kg) was also used as a positive control). In a separate study, a significant survival benefit (p=0.025) was observed in animals treated with 30 mg/kg AB0023 twice weekly in combination with 5 mg/kg Paclitaxel once per week.

Our analysis of human tumors revealed a hitherto unrecognized strong expression of LOXL2 by stromal cells among different cancers. Xenograft models of primary tumorigenesis are typically poor models for the tumor microenvironment and desmoplasia apparent in human tumors, thus a number of different cell lines were evaluated to identify a model yielding tumor formation representative of LOXL2 expression in human tumors. MDA-MB-435 was chosen as a primary tumor model for analysis of anti-LOXL2 antibody AB0023, as tumors formed by these cells generated a desmoplastic reaction and share similarities with human tumors with respect to the localization of LOXL2, with secreted LOXL2 protein at the tumor-stroma interface and collagenous matrix; and are similar in that LOXL2 is expressed by fibroblasts, blood vessels and some tumor cells (FIG. 5, Panel C). LOX localization in MDA-MB-435-generated tumors was also consistent with the patterns detected in human tumors, with cytoplasmic staining of fibroblasts, a subset of tumor cells and blood vessels, and some evidence of secreted LOX associated with the matrix (FIG. 5, Panel D).

Tumors were propagated initially in the flanks of nu/nu mice then implanted into the mammary fat pad and allowed to establish. Treatment of established primary tumors with anti-LOXL2 AB0023 resulted in a 45% decrease in tumor volume across a 3 week period (p=0.001). Weaker inhibition was also observed for anti-LOX M64, with a 27% reduction in tumor volume (p=0.04). Extension of the study for an additional 2 weeks resulted in continued tumor growth. However, a statistically significant decrease in tumor volume was maintained by treatment with AB0023 (p=0.024; 33% reduction in volume; FIG. 5, Panel E) but not by treatment with anti-LOX antibody M64. Overall, these results indicate that anti-LOXL2 AB0023 was effective in reducing tumor burden for established primary tumors over a 5 week period.

### Example 18

### Inhibition of LOXL2 Significantly Reduces Stromal Activation and Inhibits Generation of the Tumor Microenvironment

To further investigate the mechanism by which anti-LOXL2 AB0023 reduced primary tumor volume, tumors covering a matched range of relative size were harvested from vehicle-treated controls, as well as from anti-LOXL2 AB0023-treated, anti-LOX M64-treated, and taxotere-treated groups at day 39 in the MDA-MB-435 established primary tumor study. Tumors were sectioned for histology and immunohistochemistry, and analyzed using a variety of antibodies for specific cellular markers. Strikingly, the composition of AB0023-treated tumors was different when compared to all other groups, including the very small tumors isolated from high-dose taxotere-treated animals (positive control group), which despite their small size were similar in composition to the much larger vehicle-treated tumors. AB0023 treated tumors lacked many significant features of the tumor microenvironment. Specifically, there was a significant reduction in collagenous matrix or desmoplasia, as demonstrated by a 61% reduction in Sirius red staining (p=0.0027; FIG. 5, Panels G, N). Associated with this was an 88% reduction (p=0.011) in the presence of activated TAFs as assessed by aSMA signal (FIG. 5, Panels K, N). No significant differences were observed for either of these markers in the anti-LOX M64 or taxotere treated tumors (FIG. 5, Panels F-N). Tumor vasculature was also significantly reduced in the anti-LOXL2 AB0023 treated tumors (74% reduction in CD31 signal, p=0.0002; FIG. 5, Panel N). While less effective, taxotere treatment also reduced the relative tumor vasculature (43% reduction in CD31 signal, p=0.023; FIG. 5, Panel N) consistent with previous reports (FIG. 5, Panel N; FIG. 11, panels B, C, D, E).

In an independent study using the MDA-MB-435 primary tumor model, AB0023 (5 mg/kg twice per week) was compared directly with BAPN (100 mg/kg daily). A similar reduction in tumor volume was observed for AB0023-treated animals after 46 days (38.4%; p=0.04; FIG. 5, Panel O). In comparison, a 19% reduction in tumor volume (not statistically significant) was observed for mice treated daily with BAPN (FIG. 5, Panel O). Importantly, analysis of the stroma and matrix in these tumors revealed that AB0023 was significantly more effective in inhibiting stromal activation and generation of tumor infrastructure. Treatment with AB0023 again resulted in reduced collagen production (47% reduction, p=0.0193) as assessed by Sirius red staining, greatly reduced fibroblast activation as determined by αSMA positivity (>90% reduction, p=0.0161), similar to the first study, whereas tumors treated with BAPN contained desmoplastic matrix and activated fibroblasts, similar to vehicle-treated controls (FIG. 5, Panel P). Formation of vasculature was again significantly inhibited in AB0023 treated tumors (52% reduction in CD31 signal, p=0.0307) whereas there was no reduction in tumor vasculature resulting from BAPN treatment (FIG. 5, Panel P). Overall, these results confirm the effectiveness of AB0023 in inhibiting LOXL2-mediated generation of the tumor microenvironment. In comparison, the pan-LOX/L inhibitor BAPN was ineffective in inhibiting fibroblast activation, desmoplasia or angiogenesis.

Given the emerging important role of activated, tumor-associated fibroblasts in promoting tumor growth through angiogenesis, vasculogenesis and other processes, and the substantial reduction in activated fibroblasts in anti-LOXL2 AB0023 treated tumors, the effect of AB0023 treatment on expression other key factors associated with tumorigenesis was investigated. TAFs are responsible for significant VEGF production in tumors, and LOXL2 and VEGF expression patterns in human tumors share similarities in TAF-associated expression (FIG. 11, panels F, G). Analysis of MDA-MB-435 tumors revealed a 76% reduction in VEGF signal in AB0023-treated tumors compared to vehicle-treated tumors (p=0.0001; FIG. 5, Panel Q). Analysis of SDF-1/CXCL12, a pro-angiogenic and pro-tumorigenic cytokine expressed by TAFs, revealed a similar reduction in signal (80%, p=0.0205; FIG. 5, Panel Q). Levels of connective tissue growth factor (CTGF) were also reduced in tissue from AB0023-treated animals. LOXL2 signal itself was reduced by 55% (p=0.0005) in AB0023-treated tumors (FIG. 5, Panel Q; FIG. 11, panels H, I, J, K, L, M). Reductions in the levels of these growth factors, or in the level of LOXL2, were not observed in animals treated with anti-LOX antibody.

Tissue-based ELISA was used to measure the levels of transforming growth factor beta1 (TGF-β1) and of phosphorylated SMAD2 (PSMAD2) a downstream marker of TGF-β signaling. Levels of both proteins were reduced in both fibroblasts and tumor cells from AB0023-treated animals. A comparable reduction was not observed in anti-LOX treated animals or in controls that did not receive antibody. These results indicate that inhibition of LOXL2 blocks TGF-β signaling pathways in tumor tissue, leading to slower tumor growth and/or death of tumor cells.

Analysis of tumors using H&E staining and other markers indicated that fibroblasts were present in AB0023 treated tumors, although less abundant overall than in the vehicle-treated control (FIG. 11, panels N, O). This indicates that the ongoing recruitment of fibroblasts was probably also impacted by anti-LOXL2 treatment, in addition to fibroblast activation. Altogether, these data show that the inhibition of LOXL2 by AB0023 results in substantial reduction of TAF activation and fibroblast recruitment, with corresponding significant reduction in levels of key angiogenic, vasculogenic and tumor-associated factors such as VEGF and SDF-1, as well as LOXL2 itself.

Tumor cells in AB0023-treated tumors also showed differences compared to vehicle-treated tumor cells. Several tumors in the AB0023-treated group contained significant regions of necrosis (FIG. 5, Panels R, S) whereas little necrosis was apparent in other treatment groups. Furthermore, AB0023-treated tumors showed other evidence of reduced viability, with pyknosis and increased cytoplasmic condensation of nuclei consistent with early tumor necrosis, compared to the well-defined nuclei of vehicle-treated tumors (FIG. 5, Panels T, U). Levels of Beclin-1, a protein associated with autophagy, were increased in tumor cells from AB0023-treated animals and from taxotere-treated animals, but not in tumor cells from animals treated with anti-LOX antibody or in vehicle controls. These results are consistent with the idea that tumor cells in AB0023-treated animals underwent necrotic and type II autophagic cell death, resulting from the deprivation of growth factors secreted by TAFs, whose numbers are reduced in AB0023-treated animals as described supra. These analyses revealed that inhibition of LOXL2 by AB0023 was significantly more effective in reducing tumor burden, and the establishment of the tumor microenvironment, than was treatment with anti-LOX antibody M64 or the pan-LOX/L inhibitor BAPN. Inhibition of LOXL2 with a specific monoclonal antibody provides an example of a novel therapeutic strategy that targets the stromal microenvironment, which is genetically more stable than a tumor cell. The conservation of stromal LOXL2 expression patterns among multiple tumor types suggests broad applicability for the use of LOXL2 inhibitors in cancer therapy. The consequences of inhibiting LOXL2 activity extend beyond alterations of tumor infrastructure, but also include effects on the production of growth factors, pro-angiogenic proteins, and pro-vasculogenic proteins by TAFs. Thus, anti-LOXL2 therapy, while highly target-specific, has a broad spectrum of therapeutic effects that negatively impact tumor development.

### Example 19

### Anti-LOXL2 AB0023 Inhibits Liver Fibrosis and Myofibroblast Activation In Vivo

The effectiveness of anti-LOXL2 and anti-LOX antibody treatments were assessed in the context of CCl₄-induced liver fibrosis in Balb/C mice. A significant degree of mortality resulting from injection of animals with CCl₄, which was associated with liver damage and evidence of fibrogenesis (FIG. 12, panels A, B), was prevented by anti-LOXL2 antibody AB0023 but not by anti-LOX specific antibody M64 (AB0023 survival benefit p=0.0029 by log rank test and p=0.0064 by Mantel-Cox test, FIG. 6, Panel A). Analysis of the livers of surviving animals from all groups revealed that AB0023 had significantly inhibited bridging fibrosis (p=0.002, FIG. 6, Panel B; FIG. 12, Panels C, D) whereas treatment with anti-LOX M64, while showing a trend for reduction in bridging fibrosis, did not meet statistical significance (p=0.127). The porto-portal septa of vehicle-treated (FIG. 6, Panel C) and M64-treated animals contained significant populations of aSMA-positive myofibroblasts associated with bridging fibrosis. In keeping with the lack of bridging fibrosis in AB0023-treated animals, there was substantial reduction in aSMA positive myofibroblasts in porto-portal septa (FIG. 6, Panels C, D) of livers from AB0023-treated animals, indicating that AB0023 had inhibited the CCl₄-induced activation of disease-associated fibroblasts. FIG. 6, Panel e provides a quantitative analysis of α-SMA signal, demonstrating that lack of bridging fibrosis in the livers of AB0023-treated animals was accompanied by a significant reduction in the number of alpha-SMA positive myofibroblasts (p=0.0260). These results are consistent with a requirement for LOXL2 for the activation of myofibroblasts in vivo, similar to the observations presented above with respect to stromal TAFs.

### REFERENCES

Akagawa, H., A. Narita, et al. (2007). “Systematic screening of lysyl oxidase-like (LOXL) family genes demonstrates that LOXL2 is a susceptibility gene to intracranial aneurysms.” Hum Genet. 121(3-4): 377-87.
Akiri, G., E. Sabo, et al. (2003). “Lysyl oxidase-related protein-1 promotes tumor fibrosis and tumor progression in vivo.” Cancer Res 63(7): 1657-66.
Asuncion, L., B. Fogelgren, et al. (2001). “A novel human lysyl oxidase-like gene (LOXL4) on chromosome 10q24 has an altered scavenger receptor cysteine rich domain.” Matrix Biol 20(7): 487-91.
Atsawasuwan, P., Y. Mochida, et al. (2008). “Lysyl oxidase binds transforming growth factor-beta and regulates its signaling via amine oxidase activity.” J Biol Chem 283(49): 34229-40.
Atsawasuwan, P., Y. Mochida, et al. (2005). “Expression of lysyl oxidase isoforms in MC3T3-E1 osteoblastic cells.” Biochem Biophys Res Commun 327(4): 1042-6.
Bhowmick, N. A., E. G. Neilson, et al. (2004). “Stromal fibroblasts in cancer initiation and progression.” Nature 432(7015): 332-7.
Cardone, A., A. Tolino, et al. (1997). “Prognostic value of desmoplastic reaction and lymphocytic infiltration in the management of breast cancer.” Panminerva Med 39(3): 174-7.
Chang, Y. C., C. B. Liao, et al. (2008). “Expression of tumor suppressor p53 facilitates DNA repair but not UV-induced G2/M arrest or apoptosis in Chinese hamster ovary CHO-K1 cells.” J Cell Biochem 103(2): 528-37.
Chen, W., X. Wang, et al. (2008). “Blockage of NF-kappaB by IKKbeta- or RelA-siRNA rather than the NF-kappaB super-suppressor IkappaBalpha mutant potentiates adriamycin-induced cytotoxicity in lung cancer cells.” J Cell Biochem 105(2): 554-61.
Chichester, C. O., K. C. Palmer, et al. (1981). “Lung lysyl oxidase and prolyl hydroxylase: increases induced by cadmium chloride inhalation and the effect of beta-aminopropionitrile in rats.” Am Rev Respir Dis 124(6): 709-13.
Chioza, B. A., A. Ujfalusy, et al. (2001). “Mutations in the lysyl oxidase gene are not associated with amyotrophic lateral sclerosis.” Amyotroph Lateral Scler Other Motor Neuron Disord 2(2): 93-7.
Chu, T. J. and D. G. Peters (2008). “Serial analysis of the vascular endothelial transcriptome under static and shear stress conditions.” Physiol Genomics 34(2): 185-92.
Chu, W. K., P. M. Dai, et al. (2008). “Glycogen synthase kinase-3beta regulates DeltaNp63 gene transcription through the beta-catenin signaling pathway.” J Cell Biochem 105(2): 447-53.
Conti, J. A., T. J. Kendall, et al. (2008). “The desmoplastic reaction surrounding hepatic colorectal adenocarcinoma metastases aids tumor growth and survival via alphav integrin ligation.” Clin Cancer Res 14(20): 6405-13.
Copeland, R. A. (2005). Evaluation of enzyme inhibitors in drug discovery: a guide for medicinal chemists and pharmacologists. Hoboken, N.J., Wiley-Interscience.
Csiszar, K. (2001). “Lysyl oxidases: a novel multifunctional amine oxidase family.” Prog Nucleic Acid Res Mol Biol 70:1-32.
Csiszar, K., I. Entersz, et al. (1996). “Functional analysis of the promoter and first intron of the human lysyl oxidase gene.” Mol Biol Rep 23(2): 97-108.
Csiszar, K., S. F. Fong, et al. (2002). “Somatic mutations of the lysyl oxidase gene on chromosome 5q23.1 in colorectal tumors.” Int J Cancer 97(5): 636-42.
Decitre, M., C. Gleyzal, et al. (1998). “Lysyl oxidase-like protein localizes to sites of de novo fibrinogenesis in fibrosis and in the early stromal reaction of ductal breast carcinomas.” Lab Invest 78(2): 143-51.
Di, L. J., L. Wang, et al. (2008). “Identification of long range regulatory elements of mouse alpha-globin gene cluster by quantitative associated chromatin trap (QACT).” J Cell Biochem 105(1): 301-12.
El Mabrouk, M., H. Y. Qureshi, et al. (2008). “Interleukin-4 antagonizes oncostatin M and transforming growth factor beta-induced responses in articular chondrocytes.” J Cell Biochem 103(2): 588-97.
Erler, J. T., K. L. Bennewith, et al. (2009). “Hypoxia-induced lysyl oxidase is a Critical mediator of bone marrow cell recruitment to form the premetastatic niche.” Cancer Cell 15(1): 35-44.
Erler, J. T., K. L. Bennewith, et al. (2006). “Lysyl oxidase is essential for hypoxia-induced metastasis.” Nature 440(7088): 1222-6.
Erler, J. T. and A. J. Giaccia (2006). “Lysyl oxidase mediates hypoxic control of metastasis.” Cancer Res 66(21): 10238-41.
Fogelgren, B., N. Polgar, et al. (2005). “Cellular fibronectin binds to lysyl oxidase with high affinity and is critical for its proteolytic activation.” J Biol Chem 280(26): 24690-7.
Fong, S. F., E. Dietzsch, et al. (2007). “Lysyl oxidase-like 2 expression is increased in colon and esophageal tumors and associated with less differentiated colon tumors.” Genes Chromosomes Cancer 46(7): 644-55.
Gacheru, S, N., K. M. Thomas, et al. (1997). “Transcriptional and post-transcriptional control of lysyl oxidase expression in vascular smooth muscle cells: effects of TGF-beta 1 and serum deprivation.” J Cell Biochem 65(3): 395-407.
Gorogh, T., C. Holtmeier, et al. (2008). “Functional analysis of the 5′ flanking domain of the LOXL4 gene in head and neck squamous cell carcinoma cells.” Int J Oncol 33(5): 1091-8.
Gorogh, T., J. B. Weise, et al. (2007). “Selective upregulation and amplification of the lysyl oxidase like-4 (LOXL4) gene in head and neck squamous cell carcinoma.” J Pathol 212(1): 74-82.
Han, C., K. Lim, et al. (2008). “Regulation of Wnt/beta-catenin pathway by cPLA2alpha and PPARdelta.” J Cell Biochem 105(2): 534-45.
Hayashi, K., K. S. Fong, et al. (2004). “Comparative immunocytochemical localization of lysyl oxidase (LOX) and the lysyl oxidase-like (LOXL) proteins: changes in the expression of LOXL during development and growth of mouse tissues.” J Mol Histol 35(8-9): 845-55.
He, S., K. L. Dunn, et al. (2008). “Chromatin organization and nuclear microenvironments in cancer cells.” J Cell Biochem 104(6): 2004-15.
Hein, S., S. Y. Yamamoto, et al. (2001). “Lysyl oxidases: expression in the fetal membranes and placenta.” Placenta 22(1): 49-57.
Ho, C. Y., C. H. Wong, et al. (2008). “Perturbation of the chromosomal binding of RCC1, Mad2 and survivin causes spindle assembly defects and mitotic catastrophe.” J Cell Biochem 105(3): 835-46.
Hollosi, P., J. K. Yakushiji, et al. (2009). “Lysyl oxidase-like 2 promotes migration in noninvasive breast cancer cells but not in normal breast epithelial cells.” Int J Cancer.
Jansen, M. K. and K. Csiszar (2007). “Intracellular localization of the matrix enzyme lysyl oxidase in polarized epithelial cells.” Matrix Biol 26(2): 136-9.
Jeong, J. H., J. Y. An, et al. (2008). “Quercetin-induced ubiquitination and down-regulation of Her-2/neu.” J Cell Biochem 105(2): 585-95.
Jin, C. X., W. L. Li, et al. (2008). “Conversion of immortal liver progenitor cells into pancreatic endocrine progenitor cells by persistent expression of Pdx-1.” J Cell Biochem 104(1): 224-36.
Jourdan-Le Saux, C., O. Le Saux, et al. (1998). “The human lysyl oxidase-related gene (LOXL2) maps between markers D8S280 and D8S278 on chromosome 8p21.2-p21.3.” Genomics 51(2): 305-7.
Jourdan-Le Saux, C., O. Le Saux, et al. (2000). “The mouse lysyl oxidase-like 2 gene (mLOXL2) maps to chromosome 14 and is highly expressed in skin, lung and thymus.” Matrix Biol 19(2): 179-83.
Jourdan-Le Saux, C., H. Tronecker, et al. (1999). “The LOXL2 gene encodes a new lysyl oxidase-like protein and is expressed at high levels in reproductive tissues.” J Biol Chem 274(18): 12939-44.
Jung, S. T., M. S. Kim, et al. (2003). “Purification of enzymatically active human lysyl oxidase and lysyl oxidase-like protein from Escherichia coli inclusion bodies.” Protein Expr Purif 31(2): 240-6.
Kagan, H. M., V. B. Reddy, et al. (1995). “Catalytic properties and structural components of lysyl oxidase.” Ciba Found Symp 192: 100-15; discussion 115-21.
Kaku, M., Y. Mochida, et al. (2007). “Post-translational modifications of collagen upon BMP-induced osteoblast differentiation.” Biochem Biophys Res Commun 359(3): 463-8.
Kim, M. S., S. S. Kim, et al. (2003). “Expression and purification of enzymatically active forms of the human lysyl oxidase-like protein 4.” J Biol Chem 278(52): 52071-4.
Kim, Y., C. D. Boyd, et al. (1995). “A new gene with sequence and structural similarity to the gene encoding human lysyl oxidase.” J Biol Chem 270(13): 7176-82.
Kim, Y., C. D. Boyd, et al. (1997). “A highly polymorphic (CA) repeat sequence in the human lysyl oxidase-like gene.” Clin Genet. 51(2): 131-2.
Kim, Y., S. Peyrol, et al. (1999). “Coexpression of the lysyl oxidase-like gene (LOXL) and the gene encoding type III procollagen in induced liver fibrosis.” J Cell Biochem 72(2): 181-8.
Kirschmann, D. A., E. A. Seftor, et al. (2002). “A molecular role for lysyl oxidase in breast cancer invasion.” Cancer Res 62(15): 4478-83.
Klutke, J., Q. Ji, et al. (2008). “Decreased endopelvic fascia elastin content in uterine prolapse.” Acta Obstet Gynecol Scand 87(1): 111-5.
Kresse, S. H., M. Skarn, et al. (2008). “DNA copy number changes in high-grade malignant peripheral nerve sheath tumors by array CGH.” Mol Cancer 7: 48.
Laczko, R., K. M. Szauter, et al. (2007). “Active lysyl oxidase (LOX) correlates with focal adhesion kinase (FAK)/paxillin activation and migration in invasive astrocytes.” Neuropathol Appl Neurobiol 33(6): 631-43.
Lalancette, C., D. Miller, et al. (2008). “Paternal contributions: new functional insights for spermatozoal RNA.” J Cell Biochem 104(5): 1570-9.
Lelievre, E., A. Hinek, et al. (2008). “VE-statin/egf17 regulates vascular elastogenesis by interacting with lysyl oxidases.” EMBO J 27(12): 1658-70.
Leskovac, V. (2003). Comprehensive enzyme kinetics. New York, Kluwer Academic/Plenum Pub.
Li, H., X. Fan, et al. (2007). “Tumor microenvironment: the role of the tumor stroma in cancer.” J Cell Biochem 101(4): 805-15.
Liang, S., B. Moghimi, et al. (2008). “Locus control region mediated regulation of adult beta-globin gene expression.” J Cell Biochem 105(1): 9-16.
Liao, P. C., S. K. Tan, et al. (2008). “Involvement of endoplasmic reticulum in paclitaxel-induced apoptosis.” J Cell Biochem 104(4): 1509-23.
Liao, Q. C., Y. L. Li, et al. (2008). “Inhibition of adipocyte differentiation by phytoestrogen genistein through a potential downregulation of extracellular signal-regulated kinases 1/2 activity.” J Cell Biochem 104(5): 1853-64.
Liao, R., J. Sun, et al. (2008). “MicroRNAs play a role in the development of human hematopoietic stem cells.” J Cell Biochem 104(3): 805-17.
Liu, B. F. and J. J. Liang (2008). “Confocal fluorescence microscopy study of interaction between lens MIP26/AQP0 and crystallins in living cells.” J Cell Biochem 104(1): 51-8.
Lu, H. T., Y. C. Liang, et al. (2008). “Disease-modifying effects of glucosamine HCl involving regulation of metalloproteinases and chemokines activated by interleukin-1beta in human primary synovial fibroblasts.” J Cell Biochem 104(1): 38-50.
Lucero, H. A. and H. M. Kagan (2006). “Lysyl oxidase: an oxidative enzyme and effector of cell function.” Cell Mol Life Sci 63(19-20): 2304-16.
Ma, J., F. Zeng, et al. (2008). “Characterization and functional studies of a FYVE domain-containing phosphatase in C. elegans.” J Cell Biochem 104(5): 1843-52.
Macartney-Coxson, D. P., K. A. Hood, et al. (2008). “Metastatic susceptibility locus, an 8p hot-spot for tumour progression disrupted in colorectal liver metastases: 13 candidate genes examined at the DNA, mRNA and protein level.” BMC Cancer 8: 187.
Maeshima, A. M., T. Niki, et al. (2002). “Modified scar grade: a prognostic indicator in small peripheral lung adenocarcinoma.” Cancer 95(12): 2546-54.
Maki, J. M. and K. I. Kivirikko (2001). “Cloning and characterization of a fourth human lysyl oxidase isoenzyme.” Biochem J 355(Pt 2): 381-7.
Maki, J. M., H. Tikkanen, et al. (2001). “Cloning and characterization of a fifth human lysyl oxidase isoenzyme: the third member of the lysyl oxidase-related subfamily with four scavenger receptor cysteine-rich domains.” Matrix Biol 20(7): 493-6.
McElroy, K. E., P. J. Bouchard, et al. (2000). “Implementation of a continuous, enzyme-coupled fluorescence assay for high-throughput analysis of glutamate-producing enzymes.” Anal Biochem 284(2): 382-7.
Molnar, J., K. S. Fong, et al. (2003). “Structural and functional diversity of lysyl oxidase and the LOX-like proteins.” Biochim Biophys Acta 1647(1-2): 220-4.
Molnar, J., Z. Ujfaludi, et al. (2005). “Drosophila lysyl oxidases Dmloxl-1 and Dmloxl-2 are differentially expressed and the active DmLOXL-1 influences gene expression and development.” J Biol Chem 280(24): 22977-85.
Monticone, M., Y. Liu, et al. (2004). “Gene expression profile of human bone marrow stromal cells determined by restriction fragment differential display analysis.” J Cell Biochem 92(4): 733-44.
Muller, K. C., L. Welker, et al. (2006). “Lung fibroblasts from patients with emphysema show markers of senescence in vitro.” Respir Res 7: 32.
Nagaoka, H., Y. Mochida, et al. (2008). “1,25(OH)2D3 regulates collagen quality in an osteoblastic cell culture system.” Biochem Biophys Res Commun 377(2): 674-8.
Nakken, K. E., S. Nygard, et al. (2007). “Multiple inflammatory-, tissue remodelling- and fibrosis genes are differentially transcribed in the livers of Abcb4 (−/−) mice harbouring chronic cholangitis.” Scand J Gastroenterol 42(10): 1245-55.
Orimo, A., Y. Tomioka, et al. (2001). “Cancer-associated myofibroblasts possess various factors to promote endometrial tumor progression.” Clin Cancer Res 7(10): 3097-105.
Orimo, A. and R. A. Weinberg (2006). “Stromal fibroblasts in cancer: a novel tumor-promoting cell type.” Cell Cycle 5(15): 1597-601.
Palamakumbura, A. H. and P. C. Trackman (2002). “A fluorometric assay for detection of lysyl oxidase enzyme activity in biological samples.” Anal Biochem 300(2): 245-51.
Pascal, T., F. Debacq-Chainiaux, et al. (2005). “Comparison of replicative senescence and stress-induced premature senescence combining differential display and low-density DNA arrays.” FEBS Lett 579(17): 3651-9.
Payne, S. L., B. Fogelgren, et al. (2005). “Lysyl oxidase regulates breast cancer cell migration and adhesion through a hydrogen peroxide-mediated mechanism.” Cancer Res 65(24): 11429-36.
Payne, S. L., M. J. Hendrix, et al. (2007). “Paradoxical roles for lysyl oxidases in cancer—a prospect.” J Cell Biochem 101(6): 1338-54.
Peinado, H., M. Del Carmen Iglesias-de la Cruz, et al. (2005). “A molecular role for lysyl oxidase-like 2 enzyme in snail regulation and tumor progression.” EMBO J 24(19): 3446-58.
Peinado, H., G. Moreno-Bueno, et al. (2008). “Lysyl oxidase-like 2 as a new poor prognosis marker of squamous cell carcinomas.” Cancer Res 68(12): 4541-50.
Peinado, H., F. Portillo, et al. (2005). “Switching on-off Snail: LOXL2 versus GSK3beta.” Cell Cycle 4(12): 1749-52.
Pires Martins, R., R. E. Leach, et al. (2001). “Whole-body gene expression by data mining.” Genomics 72(1): 34-42.
Polgar, N., B. Fogelgren, et al. (2007). “Lysyl oxidase interacts with hormone placental lactogen and synergistically promotes breast epithelial cell proliferation and migration.” J Biol Chem 282(5): 3262-72.
Postovit, L. M., D. E. Abbott, et al. (2008). “Hypoxia/reoxygenation: a dynamic regulator of lysyl oxidase-facilitated breast cancer migration.” J Cell Biochem 103(5): 1369-78.
Qi, Y. J., Q. Y. He, et al. (2008). “Proteomic identification of malignant transformation-related proteins in esophageal squamous cell carcinoma.” J Cell Biochem 104(5): 1625-35.
Qiu, J., H. Q. Gao, et al. (2008). “Proteomics investigation of protein expression changes in ouabain induced apoptosis in human umbilical vein endothelial cells.” J Cell Biochem 104(3): 1054-64.
Rost, T., V. Pyritz, et al. (2003). “Reduction of LOX- and LOXL2-mRNA expression in head and neck squamous cell carcinomas.” Anticancer Res 23(2B): 1565-73.
Salnikow, K., O. Aprelikova, et al. (2008). “Regulation of hypoxia-inducible genes by ETS1 transcription factor.” Carcinogenesis 29(8): 1493-9.
Schlotzer-Schrehardt, U., F. Pasutto, et al. (2008). “Genotype-correlated expression of lysyl oxidase-like 1 in ocular tissues of patients with pseudoexfoliation syndrome/glaucoma and normal patients.” Am J Pathol 173(6): 1724-35.
Schmidt, H., A. Semjonow, et al. (2007). “[Mapping of a deletion interval on 8p21-22 in prostate cancer by gene dosage PCR].” Verh Dtsch Ges Pathol 91: 302-7.
Sebban, S., B. Davidson, et al. (2009). “Lysyl oxidase-like 4 is alternatively spliced in an anatomic site-specific manner in tumors involving the serosal cavities.” Virchows Arch 454(1): 71-9.
Shen, W., K. Liu, et al. (2008). “Protective effects of R-alpha-lipoic acid and acetyl-L-carnitine in MIN6 and isolated rat islet cells chronically exposed to oleic acid.” J Cell Biochem 104(4): 1232-43.
Stein, G. S., S. K. Zaidi, et al. (2008). “Genetic and epigenetic regulation in nuclear microenvironments for biological control in cancer.” J Cell Biochem 104(6): 2016-26.
Sun, J., G. Watkins, et al. (2008). “Deregulation of cofactor of BRCA1 expression in breast cancer cells.” J Cell Biochem 103(6): 1798-807.
Szabo, Z., E. Light, et al. (1997). “The human lysyl oxidase-like gene maps between STS markers D155215 and GHLC.GCT7C09 on chromosome 15.” Hum Genet 101(2): 198-200.
Szauter, K. M., T. Cao, et al. (2005). “Lysyl oxidase in development, aging and pathologies of the skin.” Pathol Biol (Paris) 53(7): 448-56.
Tang, S. S., D. E. Simpson, et al. (1984). “Beta-substituted ethylamine derivatives as suicide inhibitors of lysyl oxidase.” J Biol Chem 259(2): 975-9.
Tang, S. S., P. C. Trackman, et al. (1983). “Reaction of aortic lysyl oxidase with beta-aminopropionitrile.” J Biol Chem 258(7): 4331-8.
Trackman, P. C. and H. M. Kagan (1979). “Nonpeptidyl amine inhibitors are substrates of lysyl oxidase.” J Biol Chem 254(16): 7831-6.
Trackman, P. C., C. G. Zoski, et al. (1981). “Development of a peroxidase-coupled fluorometric assay for lysyl oxidase.” Anal Biochem 113(2): 336-42.
Tsai, C. S., F. Y. Lin, et al. (2008). “Cilostazol attenuates MCP-1 and MMP-9 expression in vivo in LPS-administrated balloon-injured rabbit aorta and in vitro in LPS-treated monocytic THP-1 cells.” J Cell Biochem 103(1): 54-66.
Urban, Z., O. Agapova, et al. (2007). “Population differences in elastin maturation in optic nerve head tissue and astrocytes.” Invest Ophthalmol V is Sci 48(7): 3209-15.
Vadasz, Z., O. Kessler, et al. (2005). “Abnormal deposition of collagen around hepatocytes in Wilson's disease is associated with hepatocyte specific expression of lysyl oxidase and lysyl oxidase like protein-2.” J Hepatol 43(3): 499-507.
Wang, Q. R., B. H. Wang, et al. (2008). “Purification and growth of endothelial progenitor cells from murine bone marrow mononuclear cells.” J Cell Biochem 103(1): 21-9.
Wang, X. M., J. Li, et al. (2008). “Involvement of the role of Chk1 in lithium-induced G2/M phase cell cycle arrest in hepatocellular carcinoma cells.” J Cell Biochem 104(4): 1181-91.
Weise, J. B., P. Rudolph, et al. (2008). “LOXL4 is a selectively expressed candidate diagnostic antigen in head and neck cancer.” Eur J Cancer 44(9): 1323-31.
Wu, M., Q. Chen, et al. (2008). “LRRC4 inhibits human glioblastoma cells proliferation, invasion, and proMMP-2 activation by reducing SDF-1 alpha/CXCR4-mediated ERK1/2 and Akt signaling pathways.” J Cell Biochem 103(1): 245-55.
Yang, H., K. R. Landis-Piwowar, et al. (2008). “Pristimerin induces apoptosis by targeting the proteasome in prostate cancer cells.” J Cell Biochem 103(1): 234-44.
Yang, Y. L., S. Y. Chang, et al. (2008). “Safflower extract: a novel renal fibrosis antagonist that functions by suppressing autocrine TGF-beta.” J Cell Biochem 104(3): 908-19.
Yu, Z., W. Li, et al. (2008). “p21 is required for atRA-mediated growth inhibition of MEPM cells, which involves RAR.” J Cell Biochem 104(6): 2185-92.
Zhang, Y., M. Q. Hassan, et al. (2008). “Intricate gene regulatory networks of helix-loop-helix (HLH) proteins support regulation of bone-tissue related genes during osteoblast differentiation.” J Cell Biochem 105(2): 487-96.
Zhao, H., Y. Liang, et al. (2008). “N-glycosylation affects the adhesive function of E-Cadherin through modifying the composition of adherens junctions (AJs) in human breast carcinoma cell line MDA-MB-435.” J Cell Biochem 104(1): 162-75.
Zheng, M., X. Gu, et al. (2008). “UBE1DC1, an ubiquitin-activating enzyme, activates two different ubiquitin-like proteins.” J Cell Biochem 104(6): 2324-34.

What is claimed is:

1. A method for inhibiting fibroblast activation in a tumor environment, the method comprising inhibiting the activity of lysyl oxidase-like 2 (LOXL2).

2. The method of claim 1, wherein the fibroblast activation is mediated by transforming growth factor-beta (TGF-β) signaling.

3. The method of claim 1, wherein inhibition of LOXL2 activity results in disorganization of the extracellular matrix.

4. The method of claim 3, wherein disorganization of the extracellular matrix results in disruption of the cytoskeleton of cells in the tumor stroma.

5. The method of claim 1, wherein the fibroblasts are tumor-associated fibroblasts (TAFs).

6. The method of claim 1, wherein the fibroblasts are myofibroblasts.

7. A method for inhibiting desmoplasia in a tumor environment, the method comprising inhibiting the activity of lysyl oxidase-like 2 (LOXL2).

8. The method of claim 7, wherein the tumor is a metastatic tumor.

9. A method for inhibiting vasculogenesis in a tumor environment, the method comprising inhibiting the activity of lysyl oxidase-like 2 (LOXL2).

10. The method of claim 9, wherein vasculogenesis comprises recruitment of vascular cells or vascular cell progenitors to a tumor environment.

11. The method of claim 9, wherein vasculogenesis comprises vascular branching.

12. The method of claim 9, wherein vasculogenesis comprises increase in vessel length.

13. The method of claim 9, wherein vasculogenesis comprises an increase in the number of vessels.

14. A method for reducing the number of tumor-associated fibroblasts (TAFs) in a tumor stroma, the method comprising inhibiting the activity of lysyl oxidase-like 2 (LOXL2).

15. A method for inhibiting collagen deposition in a tumor environment, the method comprising inhibiting the activity of lysyl oxidase-like 2 (LOXL2).

16. A method for modulating a tumor environment, the method comprising inhibiting the activity of lysyl oxidase-like 2 (LOXL2).

17. The method of claim 16, wherein modulation comprises a reduction in desmoplasia.

18. The method of claim 16, wherein modulation comprises a reduction in the number of tumor-associated fibroblasts (TAFs).

19. The method of claim 16, wherein modulation comprises a reduction in the number of myofibroblasts.

20. The method of claim 16, wherein modulation comprises remodeling of the cytoskeleton of a cell.

21. The method of claim 20, wherein the cell is a tumor cell.

22. The method of claim 20, wherein the cell is a fibroblast.

23. The method of claim 20, wherein the cell is an endothelial cell.

24. The method of claim 16, wherein modulation comprises a reduction in tumor vasculature.

25. The method of claim 16, wherein modulation comprises a reduction in collagen production.

26. The method of claim 16, wherein modulation comprises a reduction in fibroblast activation.

27. The method of claim 16, wherein modulation comprises inhibition of recruitment of fibroblasts to the tumor environment.

28. The method of claim 16, wherein modulation comprises a reduction in expression of a gene encoding a stromal component.

29. The method of claim 28, wherein the stromal component is selected from the group consisting of alpha-smooth muscle actin, Type I collagen, vimentin, matrix metalloprotease 9, and fibronectin.

30. A method for modulating the production of growth factors in a tumor environment, the method comprising inhibiting the activity of lysyl oxidase-like 2 (LOXL2).

31. The method of claim 30, wherein the growth factor is selected from the group consisting of vascular endothelial growth factor (VEGF) and stromal cell-derived factor-1 (SDF-1).

32. A method for increasing necrosis in a tumor, the method comprising inhibiting the activity of lysyl oxidase-like 2 (LOXL2).

33. A method for increasing pyknosis in a tumor, the method comprising inhibiting the activity of lysyl oxidase-like 2 (LOXL2).

34. The method of any of claims 1, 7, 9, 14, 15, 16, 30, 32 or 33, wherein the activity of LOXL2 is inhibited using an anti-LOXL2 antibody.

35. The method of claim 34, wherein the antibody comprises heavy chain sequences as set forth in SEQ ID NO:1 and light chain sequences as set forth in SEQ ID NO:2.

36. The method of claim 34, wherein the antibody is a humanized antibody.

37. The method of claim 36, wherein the antibody comprises heavy chain sequences as set forth in SEQ ID NO:3 and light chain sequences as set forth in SEQ ID NO:4.

38. The method of any of claims 1, 7, 9, 14, 15, 16, 30, 32 or 33, wherein the activity of LOXL2 is inhibited using a nucleic acid.

39. The method of claim 38, wherein the nucleic acid is a siRNA.

40. A method for identifying an inhibitor of LOXL2, the method comprising assaying a test molecule for its ability to modulate a tumor environment.

41. The method of claim 40, wherein modulation comprises a reduction in desmoplasia.

42. The method of claim 40, wherein modulation comprises a reduction in the number of tumor-associated fibroblasts (TAFs).

43. The method of claim 40, wherein modulation comprises a reduction in the number of myofibroblasts.

44. The method of claim 40, wherein modulation comprises remodeling of the cytoskeleton of a cell.

45. The method of claim 44, wherein the cell is a tumor cell.

46. The method of claim 44, wherein the cell is a fibroblast.

47. The method of claim 44, wherein the cell is an endothelial cell.

48. The method of claim 40, wherein modulation comprises a reduction in tumor vasculature.

49. The method of claim 48, wherein reduction in tumor vasculature is evidenced by reduction in the levels of CD31 and/or vascular endothelial growth factor (VEGF).

50. The method of claim 40, wherein modulation comprises a reduction in collagen production and/or a reduction in degree of collagen crosslinking.

51. The method of claim 40, wherein modulation comprises a reduction in fibroblast activation.

52. The method of claim 40, wherein modulation comprises inhibition of recruitment of fibroblasts to the tumor environment.

53. The method of claim 40, wherein modulation comprises a reduction in expression of a gene encoding a stromal component.

54. The method of claim 53, wherein the stromal component is selected from the group consisting of alpha-smooth muscle actin, Type I collagen, vimentin, matrix metalloprotease 9, and fibronectin.

55. The method of claim 40, wherein modulation comprises reduction in the levels of stromal cell-derived factor-1 (SDF-1) in the tumor environment.

56. The method of claim 40, wherein modulation comprises an increase in the incidence of necrosis and/or pyknosis in cells of the tumor.

57. The method of claim 40, wherein the test molecule is a small organic molecule with a molecular weight less than 1 kD.

58. The method of claim 40, wherein the test molecule is a polypeptide.

59. The method of claim 58, wherein the polypeptide is an antibody.

60. The method of claim 40, wherein the test molecule is a nucleic acid.

61. The method of claim 60, wherein the nucleic acid is a siRNA.


SEQUENCE LISTING (80 sequences)

SEQ ID NO: 1 - protein, 135 aa, Mus musculus
  Heavy chain
MEWSRVFIFLLSVTAGVHSQVQLQQSGAELVRPGTSVKVSCKASGYAFTYYLIEWVKQRPGQGLEWIGVINPGSGGTNYNEKFKGKATLTADKSSSTAYMQLSSLTSDDSAVYFCARNWMNFDYWGQGTTLTVSS

SEQ ID NO: 2 - protein, 132 aa, Mus musculus
  Light chain
MRCLAEFLGLLVLWIPGAIGDIVMTQAAPSVSVTPGESVSISCRSSKSLLHSNGNTYLYWFLQRPGQSPQFLIYRMSNLASGVPDRFSGSGSGTAFTLRISRVEAEDVGVYYCMQHLEYPYTFGGGTKLEIK

SEQ ID NO: 3 - protein, 116 aa, Mus musculus
  Humanized heavy chain
QVQLVQSGAEVKKPGASVKVSCKASGYAFTYYLIEWVRQAPGQGLEWIGVINPGSGGTNYNEKFKGRATITADKSTSTAYMELSSLRSEDTAVYFCARNWMNFDYWGQGTTVTVSS

SEQ ID NO: 4 - protein, 112 aa, Mus musculus
  Humanized light chain
DIVMTQTPLSLSVTPGQPASISCRSSKSLLHSNGNTYLYWFLQKPGQSPQFLIYRMSNLASGVPDRFSGSGSGTDFTLKISRVEAEDVGVYYCMQHLEYPYTFGGGTKVEIK

SEQ ID NO: 5 - DNA, 20 bp, Artificial Sequence
  Synthetic primer
atggagatcccttttggctc

SEQ ID NO: 6 - DNA, 22 bp, Artificial Sequence
  Synthetic primer
ttactgcacagagagctgatta

SEQ ID NO: 7 - DNA, 35 bp, Artificial Sequence
  Synthetic primer
atagctagcgccaccatggagatcccttttggctc

SEQ ID NO: 8 - DNA, 35 bp, Artificial Sequence
  Synthetic primer
tatactcgagtctgcacagagagctgattatttag

SEQ ID NO: 9 - DNA, 25 bp, Artificial Sequence
  Synthetic primer
cctgtcccccctgagcctggcacag

SEQ ID NO: 10 - DNA, 30 bp, Artificial Sequence
  Synthetic primer
ttactgcggggagagctggttgttcaagag

SEQ ID NO: 11 - DNA, 35 bp, Artificial Sequence
  Synthetic primer
tataggcccagccggcccagtatgacagctggccc

SEQ ID NO: 12 - DNA, 31 bp, Artificial Sequence
  Synthetic primer
tatagcggccgcctgcggggagagctggttg

SEQ ID NO: 13 - protein, 16 aa, Artificial Sequence
  Synthetic peptide
DTYERPRPGGRYRPGC

SEQ ID NO: 14 - RNA, 25 nt, Artificial Sequence
  Synthetic oligonucleotide
uaugcuuuccggaaucucgaggguc

SEQ ID NO: 15 - RNA, 25 nt, Artificial Sequence
  Synthetic oligonucleotide
uggaguaaucggauucugcaaccuc

SEQ ID NO: 16 - RNA, 25 nt, Artificial Sequence
  Synthetic oligonucleotide
ucaacgaauugucaaauuugaaccc

SEQ ID NO: 17 - RNA, 25 nt, Artificial Sequence
  Synthetic oligonucleotide
auaacagccaggacucaaucccugu

SEQ ID NO: 18 - DNA, 20 bp, Artificial Sequence
  Synthetic primer
cttgactggggaagggtctg

SEQ ID NO: 19 - DNA, 20 bp, Artificial Sequence
  Synthetic primer
aaaacggggctcaaatcacg

SEQ ID NO: 20 - DNA, 24 bp, Artificial Sequence
  Synthetic probe
atcccacccttggcattgcttggt

SEQ ID NO: 21 - DNA, 20 bp, Artificial Sequence
  Synthetic primer
agcagacttcctccccaacc

SEQ ID NO: 22 - DNA, 22 bp, Artificial Sequence
  Synthetic primer
cagtaggtcgtagtggctgaac

SEQ ID NO: 23 - DNA, 23 bp, Artificial Sequence
  Synthetic probe
cacggcacacctgggagtggcac

SEQ ID NO: 24 - DNA, 20 bp, Artificial Sequence
  Synthetic primer
ggggtttgtccacagagctg

SEQ ID NO: 25 - DNA, 20 bp, Artificial Sequence
  Synthetic primer
acgtgtcactggagaagagc

SEQ ID NO: 26 - DNA, 24 bp, Artificial Sequence
  Synthetic probe
tggagcagcaccaagagccagtct

SEQ ID NO: 27 - DNA, 20 bp, Artificial Sequence
  Synthetic primer
gtgtgcgacaaaggctggag

SEQ ID NO: 28 - DNA, 20 bp, Artificial Sequence
  Synthetic primer
ccgcgttgaccctcttttcg

SEQ ID NO: 29 - DNA, 23 bp, Artificial Sequence
  Synthetic probe
aagcccagcatcccgcagaccac

SEQ ID NO: 30 - DNA, 23 bp, Artificial Sequence
  Synthetic primer
cttaccacacacatgggtgtttc

SEQ ID NO: 31 - DNA, 22 bp, Artificial Sequence
  Synthetic primer
tcaagcactccgtaactgttgg

SEQ ID NO: 32 - DNA, 24 bp, Artificial Sequence
  Synthetic probe
ccttggaagcacagacctcgggca

SEQ ID NO: 33 - DNA, 19 bp, Artificial Sequence
  Synthetic primer
ctatccaggcggtgctgtc

SEQ ID NO: 34 - DNA, 19 bp, Artificial Sequence
  Synthetic primer
atgatggcatggggcaagg

SEQ ID NO: 35 - DNA, 24 bp, Artificial Sequence
  Synthetic probe
cctctggacgcacaactggcatcg

SEQ ID NO: 36 - DNA, 22 bp, Artificial Sequence
  Synthetic primer
tgggagtttcctgagggttttc

SEQ ID NO: 37 - DNA, 22 bp, Artificial Sequence
  Synthetic primer
gcatcttggttggctgcatatg

SEQ ID NO: 38 - DNA, 24 bp, Artificial Sequence
  Synthetic probe
agggctgcacattgcctgttctgc

SEQ ID NO: 39 - DNA, 20 bp, Artificial Sequence
  Synthetic primer
caggcaaagcaggagtccac

SEQ ID NO: 40 - DNA, 23 bp, Artificial Sequence
  Synthetic primer
cttcaacggcaaagttctcttcc

SEQ ID NO: 41 - DNA, 24 bp, Artificial Sequence
  Synthetic probe
accggagacaggtgcagtccctca

SEQ ID NO: 42 - DNA, 21 bp, Artificial Sequence
  Synthetic primer
tcaagatgcacatccgaagcc

SEQ ID NO: 43 - DNA, 20 bp, Artificial Sequence
  Synthetic primer
cagtggggacaggagaaggg

SEQ ID NO: 44 - DNA, 22 bp, Artificial Sequence
  Synthetic probe
cctgcgtctgcggaacctgcgg

SEQ ID NO: 45 - DNA, 20 bp, Artificial Sequence
  Synthetic primer
acagaacggcctcaggtacc

SEQ ID NO: 46 - DNA, 23 bp, Artificial Sequence
  Synthetic primer
ttcttggtctcgtcacagatcac

SEQ ID NO: 47 - DNA, 23 bp, Artificial Sequence
  Synthetic probe
cgtgtggaaacccgagccctgcc

SEQ ID NO: 48 - DNA, 19 bp, Artificial Sequence
  Synthetic primer
ccggctgctcagaagatac

SEQ ID NO: 49 - DNA, 23 bp, Artificial Sequence
  Synthetic primer
ttcaggtacaggctgtgatacat

SEQ ID NO: 50 - DNA, 26 bp, Artificial Sequence
  Synthetic probe
tggcgatcgatcttcttagattcacg

SEQ ID NO: 51 - DNA, 21 bp, Artificial Sequence
  Synthetic primer
caagagggaagcagagccttc

SEQ ID NO: 52 - DNA, 24 bp, Artificial Sequence
  Synthetic primer
gcaccttctgaatgtaagagtctc

SEQ ID NO: 53 - DNA, 24 bp, Artificial Sequence
  Synthetic probe
accaaggagcacgcaccacaacga

SEQ ID NO: 54 - DNA, 20 bp, Artificial Sequence
  Synthetic primer
ggccttcgccaccacctatc

SEQ ID NO: 55 - DNA, 22 bp, Artificial Sequence
  Synthetic primer
gtagtacacgtagccctgttcg

SEQ ID NO: 56 - DNA, 24 bp, Artificial Sequence
  Synthetic probe
ccagccatcctcctacccgcagca

SEQ ID NO: 57 - DNA, 22 bp, Artificial Sequence
  Synthetic primer
gctatgtagaggccaagtcctg

SEQ ID NO: 58 - DNA, 20 bp, Artificial Sequence
  Synthetic primer
cagtgacaccccagccattg

SEQ ID NO: 59 - DNA, 24 bp, Artificial Sequence
  Synthetic probe
tcctcctacggtccaggcgaaggc

SEQ ID NO: 60 - DNA, 20 bp, Artificial Sequence
  Synthetic primer
aacggcaagctgtctggaag

SEQ ID NO: 61 - DNA, 22 bp, Artificial Sequence
  Synthetic primer
agccaacattgacctagcactg

SEQ ID NO: 62 - DNA, 24 bp, Artificial Sequence
  Synthetic probe
tcccgcccattcccacccatctcg

SEQ ID NO: 63 - DNA, 24 bp, Artificial Sequence
  Synthetic primer
caagacaggtccagtagagttagg

SEQ ID NO: 64 - DNA, 23 bp, Artificial Sequence
  Synthetic primer
aggtcttataccacctgagcaag

SEQ ID NO: 65 - DNA, 24 bp, Artificial Sequence
  Synthetic probe
acagagcacagccgcctcactgga

SEQ ID NO: 66 - DNA, 21 bp, Artificial Sequence
  Synthetic primer
tctgcctctagcacacaactg

SEQ ID NO: 67 - DNA, 24 bp, Artificial Sequence
  Synthetic primer
aaaccacgagtaacaaatcaaagc

SEQ ID NO: 68 - DNA, 24 bp, Artificial Sequence
  Synthetic probe
tgtggatcagcgcctccagttcct

SEQ ID NO: 69 - DNA, 23 bp, Artificial Sequence
  Synthetic primer
cacctctgctttcttttgccatc

SEQ ID NO: 70 - DNA, 21 bp, Artificial Sequence
  Synthetic primer
ctgtgggaggggtgtttgaac

SEQ ID NO: 71 - DNA, 24 bp, Artificial Sequence
  Synthetic probe
tgcagcactgtcaggacatggcct

SEQ ID NO: 72 - DNA, 20 bp, Artificial Sequence
  Synthetic primer
cgccctcattcccttgttgc

SEQ ID NO: 73 - DNA, 20 bp, Artificial Sequence
  Synthetic primer
ggaggacgaggacacagacc

SEQ ID NO: 74 - DNA, 22 bp, Artificial Sequence
  Synthetic probe
ttccagccgcagcaagccagcc

SEQ ID NO: 75 - DNA, 18 bp, Artificial Sequence
  Synthetic primer
cggctgtgtgcgatgacg

SEQ ID NO: 76 - DNA, 22 bp, Artificial Sequence
  Synthetic primer
acgtattcttccgggcagaaag

SEQ ID NO: 77 - DNA, 24 bp, Artificial Sequence
  Synthetic probe
cagcactcgccctcccgtctttgg

SEQ ID NO: 78 - DNA, 21 bp, Artificial Sequence
  Synthetic primer
agaaggtgacctggatgagaa

SEQ ID NO: 79 - DNA, 22 bp, Artificial Sequence
  Synthetic primer
tgatacatatggcggtcaatct

SEQ ID NO: 80 - DNA, 27 bp, Artificial Sequence
  Synthetic probe
cttctcaggagataccgggaatccaag